(19) United States
(12) Patent Application Publication
(10) Pub. No.: US 2017/0014517 A1
(43) Pub. Date: Jan. 19, 2017

(54) VEHICLE COMPOSITIONS ESSENTIALLY FREE OF PHARMACEUTICALLY ACTIVE AGENTS FOR THE IMPROVED TREATMENT OF ACNE AND RELATED DISORDERS

(71) Applicant: Foamix Pharmaceuticals Ltd., Rehovot (IL)

(72) Inventors: Dov Tamarkin, Ness Ziona (IL); Elana Gazal, Rehovot (IL); Rita Keynan, Rehovot (IL); Meir Eini, Ness Ziona (IL); David Schuz, Gimzu (IL)

(21) Appl. No.: 14/858,388

(22) Filed: Sep. 18, 2015

Related U.S. Application Data
(63) Continuation of application No. 14/147,401, filed on Jan. 3, 2014, now abandoned.
Application No. 14/147,401 is a continuation-in-part of application No. 13/831,396, filed on Mar. 14, 2013.
Application No. 13/831,396 is a continuation-in-part of application No. PCT/IB2013/001170, filed on Mar. 14, 2013.
Application No. PCT/IB2013/001170 is a continuation-in-part of application No. 13/499,501, filed on Sep. 10, 2012, now Pat. No. 8,945,516.
Application No. 13/499,501 is a continuation-in-part of application No. 13/100,724, filed on May 4, 2011, now Pat. No. 8,618,081.
Application No. 13/100,724 is a continuation-in-part of application No. PCT/IB2010/002612, filed on Oct. 1, 2010.
Application No. 13/100,724 is a continuation-in-part of application No. PCT/IB2010/002617, filed on Oct. 1, 2010.
Application No. 13/100,724 is a continuation-in-part of application No. PCT/IB2010/002613, filed on Oct. 1, 2010.
(60) Provisional application No. 61/748,603, filed on Jan. 3, 2013.
(60) Provisional application No. 61/780,074, filed on Mar. 13, 2013.
(60) Provisional application No. 61/779,953, filed on Mar. 13, 2013.
(60) Provisional application No. 61/248,144, filed on Oct. 2, 2009.
(60) Provisional application No. 61/322,148, filed on Apr. 8, 2010.
(60) Provisional application No. 61/349,911, filed on May 31, 2010.
(60) Provisional application No. 61/385,385, filed on Sep. 22, 2010.
(60) Provisional application No. 61/331,126, filed on May 4, 2010.
(60) Provisional application No. 61/380,568, filed on Sep. 7, 2010.
(60) Provisional application No. 61/248,144, filed on Oct. 2, 2009.
(60) Provisional application No. 61/322,148, filed on Apr. 8, 2010.
(60) Provisional application No. 61/349,911, filed on May 31, 2010.
(60) Provisional application No. 61/385,385, filed on Sep. 22, 2010.
(60) Provisional application No. 61/331,126, filed on May 4, 2010.
(60) Provisional application No. 61/388,884, filed on Oct. 1, 2010.
(60) Provisional application No. 61/380,568, filed on Sep. 7, 2010.
(60) Provisional application No. 61/248,144, filed on Oct. 2, 2009.
(60) Provisional application No. 61/322,148, filed on Apr. 8, 2010.
(60) Provisional application No. 61/349,911, filed on May 31, 2010.
(60) Provisional application No. 61/385,385, filed on Sep. 22, 2010.
(60) Provisional application No. 61/331,126, filed on May 4, 2010.
(60) Provisional application No. 61/388,884, filed on Oct. 1, 2010.
(60) Provisional application No. 61/380,568, filed on Sep. 7, 2010.
(60) Provisional application No. 61/248,144, filed on Oct. 2, 2009.
(60) Provisional application No. 61/322,148, filed on Apr. 8, 2010.
(60) Provisional application No. 61/349,911, filed on May 31, 2010.
(60) Provisional application No. 61/385,385, filed on Sep. 22, 2010.
(60) Provisional application No. 61/331,126, filed on May 4, 2010.
(60) Provisional application No. 61/388,884, filed on Oct. 1, 2010.
(60) Provisional application No. 61/380,568, filed on Sep. 7, 2010.

(51) Int. Cl. A61K 47/44; A61K 47/12; A61K 47/10

(52) U.S. Cl. CPC A61K 47/44; A61K 47/10; A61K 47/12

(57) ABSTRACT

The present invention related to the use of a pharmaceutical composition which is essentially free of pharmaceutically active agents for the treatment of human skin, especially in the treatment of acne, acne related symptoms, a tetracycline antibiotic responsive acne related disorder, skin disorder caused by a bacteria, and a tetracycline antibiotic responsive sebaceous gland disease, P. acne bacteria associated disorders, and other superficial infections, including skin infections.

### CROSS REFERENCE TO RELATED APPLICATIONS

This application is a continuation of U.S. patent application Ser. No. 14/147,401, filed Jan. 3, 2014, which is a continuation-in-part application of: U.S. patent application Ser. No. 13/831,396, filed Mar. 14, 2013 and International Patent Application No. PCT/IB2013/001170, filed Mar. 14, 2013, which claims the benefit of priority under 35 U.S.C. §119(e) to U.S. Provisional Application No. 61/748,603, filed Jan. 3, 2013, U.S. Provisional Application No. 61/780,074, filed Mar. 13, 2013, U.S. Provisional Application No. 61/779,953, filed Mar. 13, 2013, U.S. Provisional Application No. 61/831,981, filed Jun. 6, 2013, and U.S. Provisional Application No. 61/831,795, filed Jun. 6, 2013; all of which are incorporated by reference in their entireties.

### BACKGROUND

Acne, including acne vulgaris and acne-rosacea (also termed “rosacea”) are skin diseases which involve infected lesions, including non-inflammatory and inflammatory lesions. Non-inflammatory acne lesions include blackheads (open comedones) and whiteheads (closed comedones). Open and closed comedones along with papules and pustules are referred to as papulopustular acne, a form of inflammatory acne. The more severe the disease is, it involves more infected, inflammatory lesions. Nodular acne is the most severe form of inflammatory acne. If improperly treated, inflammatory acne lesions can produce deep scarring. Commonly used antibiotics either applied topically or taken orally, include erythromycin, clindamycin, and tetracyclines such as minocycline.

Zindaclin 1% Gel (CLINDAMYCIN PHOSPHATE, Crawford Pharmaceuticals) treats only mild to moderate acne and frequently causes side effects. Evoclin® Foam1%, contains clindamycin phosphate, in a thermolabile hydroethanolic foam vehicle. It is indicated once daily topically for twelve weeks for treating mild to moderate acne. Evoclin® Foam side effects include burning, itching, dryness, redness, oily skin or skin peeling.

Akne-mycin®2% is in the form of an ointment (erythromycin, CORIA LABORATORIES) indicated twice daily may cause erythema and peeling.

Minocycline hydrochloride is a bacteriostatic tetracycline antibiotic, which has a broader spectrum than the other members of the group. It reduces the redness, swelling and tenderness or pimples whether it kills the acne bacteria or not. It is currently available as an oral drug under the brand names Dynacin, Minocin, Myrac, Solodyn. It is further available in injectable products for IV administration.

A number of foamable compositions containing pharmaceutically active agents are known in the art for the treatment of various medical conditions of the skin or of body cavities.

In one or more embodiments there is provided a vehicle (placebo) that is essentially free of pharmaceutically active agents, and or that requires a shorter treatment period, and or has no systemic side effects or adverse events, and or does not cause skin irritation, and or is safe for use for pregnant and nursing mothers. Such a vehicle is advantageous and could improve patient compliance. The vehicle composition may have reduced efficacy in treatment of diseases as compared to the same composition containing an active pharmaceutical agent. Side effects of pharmaceuticals are a disadvantage. So there is provided herein a composition essentially free of pharmaceutically active agents. Provided herein is a composition that can reduce adverse systemic exposure as well as reduce unwanted side effects of antibiotics, but nevertheless treat both inflammatory and non-inflammatory lesions effectively. In one or more embodiments the composition is essentially free of antimicrobials. In one or more embodiments it is essentially free of antibiotics. In one or more embodiments it is essentially free of tetracycline antibiotics. In one or more embodiments it is essentially free of a minocycline. In this connection, the term “vehicle” is used to refer to and is synonymous with a composition that is essentially free of pharmaceutical active agents, and/or antimicrobials, and/or antibiotics, and/or tetracycline antibiotics, and/or a minocycline. Placebo and or carrier can also be used to refer to the vehicle. Novel, stable, patient-friendly topical hydrophobic therapeutic breakable gel and foamable compositions comprising tetracycline, without surfactants, have been described, for example in U.S. application Ser. Nos. 13/499,501, 13/499,727, 13/499,475, and 13/499,709, U.S. Publication No. 2011/0281827, WO11/039637, WO11/039638, WO 11/138678 and WO 2011/064631, all of which are herein incorporated in their entirety by reference.

Methods for treatment of impetigo using these topical hydrophobic therapeutic breakable gel and foamable compositions comprising tetracycline have been described, for example in U.S. provisional application Ser. Nos. 61/748,603 61/611,232, 61/780,074 and U.S. patent application Ser. No. 13/831,396 respectively all of which are herein incorporated in their entirety by reference. Methods for treatment of acne using these topical hydrophobic therapeutic breakable gel and foamable compositions comprising tetracycline have been described, for example in U.S. provisional application 61/779,953 and 61/831,795, and PCT/US2013/031387 also herein incorporated in their entirety by reference. Methods for treatment of acne using topical hydrophobic breakable gel and foamable compositions without tetracycline antibiotics is described, for example, in Applicants earlier provisional application Ser. No. 61/779,953, also herein incorporated in their entirety by reference.

Foamable compositions previously developed by Applicants can contain a number of pharmaceutical active agents for the treatment of a variety of diseases of the skin such impetigo, rosacea or acne. These foams arc easy to apply to the skin and avoid stinging and drying, properties that have been reported from previous foam compositions. However, all of these compositions require the presence of one or more pharmaceutically active agents such as tetracyclines.

As can be seen form the above current anti-acne preparations can cause significant adverse reactions, and their efficacy is limited.

The safety, tolerability and clinical efficacy of topical application of the vehicle of said hydrophobic breakable gel and foamable compositions in a population of moderate to severe acne vulgaris patients is assessed herein. A safe and effective dose and dosing regimen for said hydrophobic breakable gel and foamable compositions is described herein. The vehicle is essentially free or free of active pharmaceutical ingredients, such as tetracycline antibiotics.

### SUMMARY

The present invention relates to a method of treatment of a disease using a hydrophobic gel or foam vehicle that does not comprise a tetracycline antibiotic and is essentially free of pharmaceutically active agents (hereinafter “vehicle”).

In one or more embodiments, the vehicle is essentially free of a pharmaceutically active agent. In one or more embodiments the vehicle is free of a pharmaceutically active agent. In one or more embodiments the vehicle is free of a tetracycline. In one or more embodiments the vehicle is essentially free of a tetracycline. In one or more embodiments the vehicle is free of a minocycline. In one or more embodiments the vehicle is essentially free of a minocycline. It has now surprisingly been found, that a vehicle which is essentially free of pharmaceutically active agents, consisting of (a) at least one hydrophobic solvent, (b) at least one viscosity modifying agent, and which forms a foam if a propellant is added thereto that, can be used for the treatment of human skin especially for the treatment of one or more of acne, acne related symptoms, a tetracycline antibiotic responsive acne related disorder, skin disorder caused by a bacteria, a tetracycline antibiotic responsive sebaceous gland disease, P. acne bacteria associated disorders, and other superficial infections, including skin infections, rosacea, atopic dermatitis, contact dermatitis, perioral dermatitis, psoriasis and neurodermatitis

The results in a Phase II, placebo controlled and double blinded clinical study in acne patients have now surprisingly demonstrated that the vehicle, which is essentially free of pharmaceutically active agents, administered topically weeks once daily had an unexpected effect in reducing the number of inflammatory lesions and in reducing the number of non-inflammatory lesions in patients and was safe and well-tolerated. Even more surprisingly, the improvement in inflammatory lesions observed with the vehicle was not merely favorably comparable to the efficacy results for oral minocycline as presented in the Solydyn™ Patient Product leaflet at 12 weeks but showed a better efficacy as early on as after 6 weeks of treatment.

The 150 patients starting the clinical trial had on average moderate severe to severe acne with an average number of inflammatory lesions ranging from 33-36 and an average number of non-inflammatory lesions of 42-46. Accordingly, patients started the study with about 75-82 lesions on average. The effects observed were dose dependent. The results seen with 4% minocycline were better than those with 1% minocycline which in turn were better than the composition without minocycline which was used as the placebo (the “vehicle” or “placebo formulation” or “placebo”). Nevertheless, the vehicle also had a substantial and unexpected positive effect. For example, as described below, following daily application a substantial reduction of acne lesions was observed. The effect of the vehicle without minocycline may be a contributing factor in the success observed with of the 1% and 4% Minocycline formulations as it may act as a springboard or platform from which the antibiotic can have its therapeutic effect.

By having an effect on its own right, the vehicle together with an active pharmaceutical e.g. minocycline can achieve more for the skin than oral minocycline.

Clinical trial results of acne patients treated with the vehicle have now surprisingly demonstrated a reduction in both inflammatory and non-inflammatory lesions from about 3 weeks with improvement continuing to 12 weeks. Non-inflammatory lesions responded, in general, slightly better that than inflammatory lesions. The effect of treatment on reducing the number of lesions and improving the patient's skin appeared to approach a steady state between 6-12 weeks for both inflammatory lesions and non-inflammatory. Treatment was stopped at twelve weeks but the patients were seen again 4 weeks after cessation of treatment, at week 16. Surprisingly, the effect of the previous 12 weeks of treatment on reducing the number of lesions and improving the patient's skin was observed to continue in the absence of treatment with minor mean decrease in the number of lesions. In other words, four weeks after cessation the patient's skin did not appear to show signs of relapse.

Reduction of 49% and 50% in the mean number of non-inflammatory and inflammatory acne lesions respectively within only six weeks of treatment with the vehicle was demonstrated. A further mean reduction of about 57% in the number of non-inflammatory lesions and 51% in inflammatory acne lesions was observed after twelve weeks of treatment.

Unexpectedly, in the course of treatment the vehicle was able to reduce symptoms and severity of acne. Improvement was apparent as was the restoration of visible, normal coetaneous topographic features, indicating the return of skin integrity.

Without being bound by any theory, it is thought the selection of excipients that are compatible with minocycline may have contributed to the efficacy of the vehicle. Other possible theories for the efficacy of the vehicle include the application of oils to the skin. Although this may run counter to current thinking that oily material should be avoided, it appears that its presence may actually help improve the skin, and/or extract sebaceous matter from the gland/or pores and/or have a negative feedback so as to reduce the production of material that can block or interfere with the operation of the sebaceous glands. Other contributing factors may include the presence of fatty alcohols; and/or the presence of a fatty acid; and/or the presence of waxes in the vehicle.

The percentage reduction in inflammatory lesions and also in non-inflammatory lesions was found to be comparable and even better to than seen with other current acne treatments. For example application of vehicle resulted in a 51% reduction of inflammatory lesions and 57% of non-inflammatory lesions, respectively at 12 weeks. In contrast, oral minocycline (Solodyn™) efficacy results presented in the Patient Package Insert show an overall reduction of ˜44% (43.1 study 1 and 45.8 study 2) in the number of inflammatory lesions and no effect on non-inflammatory lesions. Remarkably, the reduction in the number of inflammatory lesions demonstrated with the vehicle was greater than the reduction recorded for oral minocycline. The reduction in non-inflammatory lesions demonstrated with the vehicle is greater than for four recently approved topical products which use active ingredients other than tetracycline antibiotics, namely Epiduo™, Acanya™ Fabior™ and Ziana™. So apart from the avoidance of unwanted systemic effects, topical vehicle treatment appears to have substantial advantages over oral minocycline treatment of acne and other available topical treatments.

The improvement indicated in the Investigators Global Assessment (“IGA”) (FDA: Guidance for Industry-Acne Vulgaris: Developing Drugs for Treatment, 2005, p. 3) of the vehicle was also very encouraging as shown in Example 3. The percentage of patients, for example, with an IGA at 12 weeks of “almost clear” or “clear” was 20% for the placebo. Even after treatment ceased patients IGA score continued to improve to 33%. Patient feedback was likewise positive. High overall patient satisfaction was reported.

Remarkably, no systemic side effects, no adverse events and only few skin irritation events were observed in patients treated with the vehicle during 12 weeks of treatment. All these skin irritation events were mostly mild and transient and did not require discontinuance treatment.

A mouth after the end of treatment one patient manifested mild peeling and another manifested mild skin dryness. Without being bound by any theory, since the skin irritation events did not occur during treatment they may be linked to withdrawal of the application of the vehicle, e.g. withdrawal of the application of the oil provided by the formulation.

The vehicle appears to avoid or minimize known side effects and may act to prevent or minimize or ameliorate side effects which might otherwise occur when a formulation is applied topically to the skin or mucosa, such as, skin irritation, thereby leading to better patient compliance compared to available treatment options.

Thus, the vehicle compositions provided herein offer a safe, user friendly, and effective alternative to current oral minocycline treatments and other topical treatments. Moreover, they provide a shorter treatment regime (six weeks) with comparable or even better efficacy results to available treatment options with regard to inflammatory lesion and enhanced efficacy results with regard to non-inflammatory lesion while avoiding unwanted side effects, adverse events and skin irritation.

The vehicle in one embodiment is presented as a foam. In one or more embodiments the foam is a breakable foam. In another embodiment the vehicle is presented as a gel. In some embodiments the gel is liquid, in other embodiments it is semi-solid and in still further embodiments it is stable e.g. such that if inverted it generally maintains its shape. In one or more embodiments when a mechanical or shear force is applied to the gel it becomes flowable or liquid.

Whilst the clinical trial was on the foam vehicle it is anticipated that the gel will have a similar or comparable effect to the foam. In one or more embodiments the resultant foam has the same formulation as the gel after dissipation of propellant. In one or more embodiments the gel only differs from the foam by the absence of propellant. In one or more embodiments the foam is generated from a gel in a container with an outlet valve (an aerosol canister) to which is added propellant. Upon release of the composition (gel plus propellant) from the canister the propellant dissipates and a foam is generated having the composition of the gel but in the form of a foam. Furthermore, in certain cases, when the foam is collapsed it can revert to having gel-like properties.

In one or more embodiments there is provided a method of treating or alleviating a disorder selected from the group consisting of acne, acne related symptoms, a tetracycline antibiotic responsive acne related disorder, a tetracycline antibiotic responsive skin disorder, skin disorder caused by a bacteria, a tetracycline antibiotic responsive disorder, a sebaceous gland disorder, P. acne bacteria associated disorders and other superficial infections, including skin infections, comprising administering topically at least alternate days or once daily to a target area on a human subject having the disorder a hydrophobic gel or foam vehicle essentially free of a pharmaceutically active agent wherein the target area is the skin.

In one or more embodiments there is provided a method of treating or alleviating conditions, in which inflammation, pro-inflammatory cytokines and/or apoptosis is a part of their etiological factors.

In one or more embodiments there is provided a method of treating or alleviating skin inflammation.

In one or more embodiments there is provided a method of treating or alleviating a disorder, selected from the group consisting of a dermatitis, atopic dermatitis, contact dermatitis, perioral dermatitis, stasis dermatitis, seborrheic dermatitis, rosacea, psoriasis, rash, diaper rash, light-induced burn, sun burn, chemical burn, radiation burn.

In one or more embodiments there is provided a method of treating or alleviating acne. In one or more embodiments there is provided a method of treating or alleviating acne related symptoms. In one or more embodiments there is provided a method of treating or alleviating a tetracycline antibiotic responsive acne related disorder. In one or more embodiments there is provided a method of treating or alleviating a tetracycline antibiotic responsive skin disorder. In one or more embodiments there is provided a method of treating or alleviating skin disorder caused by a bacteria. In one or more embodiments there is provided a method of treating or alleviating a tetracycline antibiotic responsive disorder. In one or more embodiments there is provided a method of treating or alleviating a sebaceous gland disorder. In one or more embodiments there is provided a method of treating or alleviating superficial infections, including skin infections.

In one or more embodiments there is provided a method of reducing the number of inflammatory lesions in a patient diagnosed with acne. In one or more embodiments there is provided a method of reducing the number of non-inflammatory lesions in a patient diagnosed with acne. In one or more embodiments there is provided a method of improving the success rate in treatment of a patient diagnosed with acne as measured by the mean percentage change of lesions. In one or more embodiments there is provided a method of improving the success rate in treatment of a patient diagnosed with acne as measured by percent of subjects who had more than 45% reduction in the number of lesions. In one or more embodiments there is provided a method of improving the success rate in treatment of a patient diagnosed with acne as measured by improving the % number of patients with an IGA of “almost clear” or “clear”. In one or more embodiments there is provided a method of improving the success rate in treatment of a patient diagnosed with acne as measured by improving the percent number of patients with an improvement of 2 grades in IGA score compared to baseline. In one or more embodiments there is provided a method of improving the success rate in treatment of a patient diagnosed with acne as measured by the percent of patients receiving an investigator improvement score of at least moderate. In one or more embodiments there is provided a method of improving the success rate in treatment of a patient diagnosed with acne as measured by the percent of patients receiving an investigator improvement score of excellent In one or more embodiments there is provided a method of improving the success rate in treatment of a patient diagnosed with acne as measured by the percent of patients who assess their global improvement as at least slightly better. In one or more embodiments there is provided a method of improving the success rate in treatment of a patient diagnosed with acne as measured by the percent of patients who assess their global improvement as at least better.

In one or more embodiments there is provided a method of preventing or delaying the reoccurrence of acne. In one or more embodiments there is provided a method of preventing or delaying the reoccurrence of acne related symptoms. In one or more embodiments there is provided a method of preventing or delaying the reoccurrence of a tetracycline antibiotic responsive acne related disorder. In one or more embodiments there is provided a method of preventing or delaying the reoccurrence of a tetracycline antibiotic responsive skin disorder. In one or more embodiments there is provided a method of preventing or delaying the reoccurrence of skin disorder caused by a bacteria. In one or more embodiments there is provided a method of preventing or delaying the reoccurrence of a tetracycline antibiotic responsive disorder. In one or more embodiments there is provided a method of preventing or delaying the reoccurrence of a sebaceous gland disorder. In one or more embodiments there is provided a method of preventing or delaying the reoccurrence of superficial infections, including skin infections.

In any one or more embodiments any of the above methods comprises applying a vehicle topically to a target area of skin on a subject diagnosed with acne. In one or more embodiments, the vehicle is an oil based carrier. In one or more embodiments the vehicle is surfactant free. In one or more embodiments the vehicle is polymeric agent free. In one or more embodiments the vehicle is ethanol free. In one or more embodiments the vehicle is free of a pharmaceutically active agent. In one or more embodiments the vehicle is essentially free of a pharmaceutically active agent. In one or more embodiments the vehicle is free of a tetracycline. In one or more embodiments the vehicle is essentially free of a tetracycline. In one or more embodiments the vehicle is free of a minocycline. In one or more embodiments the vehicle is essentially free of a minocycline. In one or more alternative embodiments the vehicle is polyol free. In one or more alternative embodiments the vehicle is hydrophilic solvent free. In one or more embodiments the vehicle comprises a hydrophobic solvent and a fatty alcohol. In one or more embodiments the vehicle comprises a hydrophobic solvent and a fatty alcohol and a wax. In one or more embodiments the vehicle comprises a hydrophobic solvent and a fatty alcohol and a wax and a fatty acid. In one or more embodiments the vehicle is a gel and/or the vehicle is a foam. In one or more embodiments the vehicle is a foamable composition.

In one or more embodiments there is provided a method of treating or alleviating a skin disorder. In one or more embodiments the hydrophobic gel or foam vehicle is free of a pharmaceutically active agent and comprises:
a) about 60% to about 99% by weight of at least one hydrophobic solvent;
b) at least one viscosity-modifying agent selected from the group consisting of a fatty alcohol, a fatty acid, and a wax;

In one or more embodiments the viscosity modifying agent comprises a wax in combination with a fatty alcohol or a fatty acid or both. In one or more embodiments the viscosity modifying agent comprises mixtures of fatty alcohols and a wax. In one or more embodiments the viscosity modifying agent comprises mixtures of fatty alcohols and waxes. In one or more embodiments the viscosity modifying agent comprises mixtures of fatty acids and a wax. In one or more embodiments the viscosity modifying agent comprises mixtures of fatty acids and waxes. In one or more embodiments the wax comprises a hydrogenated oil. In one or more embodiments the hydrogenated oil is a hydrogenated castor oil. In one or more embodiments the wax comprises a paraffin wax. In one or more embodiments the wax comprises a beeswax.

In one or more embodiments the hydrophobic foam used in the method is formed from the hydrophobic gel composition further comprising a propellant.

In an embodiment the disorder is acne vulgaris.

In an embodiment the disorder is an inflammatory disorder.

In an embodiment the disorder is an inflammatory disorder or an inflammation, or a condition, in which inflammation, pro-inflammatory cytokines and/or apoptosis is part of its etiological factors.

In an embodiment the disorder is a non-inflammatory disorder.

In an embodiment the disorder displays one or more lesions selected form the group consisting of comedonal, papulopustular, nodulocystic, and mixtures of any two or more thereof.

In one or more embodiments the hydrophobic gel or foam vehicle used in the method is applied on average at a frequency selected from the group consisting of three times daily, twice daily, once daily and alternate day.

In one or more embodiments the hydrophobic gel or foam vehicle used in the method is administered for a period selected from the group consisting of two weeks, three weeks, four weeks, five weeks, six weeks, seven weeks, eight weeks, nine weeks, ten weeks, eleven weeks, twelve weeks, thirteen weeks, fourteen weeks, fifteen weeks, and sixteen weeks. In one or embodiments the hydrophobic gel or foam vehicle used in the method is administered for a period longer than 16 weeks, for example seventeen or eighteen or nineteen, twenty, twenty-one, twenty-two, twenty-three or twenty-four weeks or such longer period as is needed to bring the disorder under control.

In one or more embodiments the hydrophobic gel or foam vehicle used in the method is applied as a maintenance dose after the therapy period at a frequency selected from the group consisting of every two days, three times a week, twice a week, once a week, once in two weeks, once in three weeks, once a month, once in two months, and alternate weeks. In one or more embodiments the maintenance dose is discontinued after a period selected from the group consisting of a week, two weeks, three weeks, four weeks, a month, two months, three months, four months, five months, and six months.

In one or more embodiments the hydrophobic vehicle foam or gel used in the method is effective against P. acne bacteria associated disorders.

In one or more embodiments of the invention, there is disclosed a method for treating a disorder of the pilosebaceous unit, or of the sebaceous gland or of the hair follicle including acne, including administering topically to a surface having acne a hydrophobic composition substantially free of surfactants, and/or substantially free of surfactants and polymeric agents as described above, wherein (a) the at least one hydrophobic solvent comprises or is selected from a group consisting of a soybean oil, a coconut oil, a cyclomethicone, a light mineral oil, and mixtures thereof; (b) the fatty alcohol comprises or is selected from a group consisting of cetostearyl alcohol, myristyl alcohol, stearyl alcohol, behenyl alcohol, and mixtures thereof; the fatty acid comprises steal is acid; and the wax comprises or is selected from the group consisting of a beeswax, a hydrogenated oil, and mixtures thereof.

It is postulated, without being bound by any theory that the use of a hydrophobic oil based foam vehicle contributes to cutaneous bioavailability, in or around the pilosebaceous unit. Specific targeting of hydrophobic oil based foam vehicle to the pilosebaceous unit is thought enabled due the hydrophobic nature of the pilosebaceous gland.

In one or more embodiments the hydrophobic vehicle foam or gel used in the method results in a decrease of at least about 40% in the number of the inflammatory acne vulgaris lesions after twelve weeks of treatment, wherein the hydrophobic foam or gel vehicle is administered once daily. In one or more embodiments the decrease is at least about 45%, at least about 50%, at least about 55%, at least about 60%, at least about 65% or at least about 70%.

In one or more embodiments the hydrophobic vehicle foam or gel used in the method results in a decrease of at least about 50% in the number of the inflammatory acne vulgaris lesions four weeks after the end of the treatment. In one or more embodiments the decrease is at least about 55%, or at least about 60%, or at least about 65%, or at least about 70%.

In one or more embodiments the hydrophobic gel or foam vehicle used in the method obtains a decrease of at least 45% in the number of inflammatory lesions after nine weeks or less than nine weeks of treatment, when the composition is administered on average once daily. In one or more embodiments the decrease is at least about 50%, or at least about 55% at least about 60%, or at least about 65%, or at least about 70%.

In one or more embodiments the hydrophobic gel or foam vehicle used in the method obtains a decrease of at least 40% in the number of inflammatory lesions after six weeks or less than six weeks of treatment, when the composition is administered on average once daily. In one or more embodiments the decrease is at least about 45%, or at least about 50%, or at least about 55% at least about 60%, or at least about 65%, or at least about 70%.

In one or more embodiments the hydrophobic gel or foam vehicle used in the method obtains at least a 20% decrease in the number of inflammatory lesion after three weeks or less than three weeks of treatment, when the vehicle is administered on average once daily. In one or more embodiments the decrease is at least about 25%, or at least about 30%, or at least about 35% at least about 40%, or at least about 45%, or at least about 50%. In one or more embodiments the hydrophobic gel or foam vehicle used in the method obtains at least a 40% mean decrease in the number of inflammatory lesion after three weeks or less than three weeks of treatment, when the vehicle is administered on average once daily.

In one or more embodiments the hydrophobic vehicle foam or gel used in the method results in a decrease of at least about 50% in the number of the non-inflammatory acne vulgaris lesions after twelve weeks of treatment, wherein the hydrophobic foam or gel vehicle is administered once daily. In one or more embodiments the decrease is at least about 55%, at least about 60%, at least about 65% or at least about 70%.

In one or more embodiments the hydrophobic vehicle foam or gel used in the method results in a decrease of at least about 50% in the number of the non-inflammatory acne vulgaris lesions four weeks after the end of the treatment. In one or more embodiments the decrease is at least about 55%, or at least about 60%, or at least about 65%, or at least about 70%.

In one or more embodiments the hydrophobic gel or foam vehicle used in the method obtains a mean decrease of at least 45% in the number of non-inflammatory lesions after nine weeks or less than nine weeks of treatment, when the composition is administered on average once daily. In one or more embodiments the decrease is at least about 50%, or at least about 55% at least about 60%, or at least about 65%.

In one or more embodiments the hydrophobic gel or foam vehicle used in the method obtains a decrease of at least 40% in the number of non-inflammatory lesions after six weeks or less than six weeks of treatment, when the composition is administered on average once daily. In one or more embodiments the decrease is at least about 45%, or at least about 50%, or at least about 55% at least about 60%.

In one or more embodiments the hydrophobic gel or foam vehicle used in the method obtains a at least a 20% decrease in the number of non-inflammatory lesion after three weeks or less than three weeks of treatment, when the vehicle is administered on average once daily. In one or more embodiments the decrease is at least about 25%, or at least about 30%, or at least about 35% at least about 40%, or at least about 45%, or at least about 50%. In one or more embodiments the hydrophobic gel or foam vehicle used in the method results in at least a 40% decrease in the number non-inflammatory lesion after three weeks or less than three weeks of treatment, when the vehicle is administered on average once daily.

In one or more embodiments the hydrophobic vehicle foam or gel used in the method results in a decrease of at least about 45% in the total number of acne vulgaris lesions (inflammatory plus non-inflammatory lesions) after twelve weeks of treatment, wherein the hydrophobic foam or gel vehicle is administered once daily. In one or more embodiments the mean decrease is at least about 50%, at least about 55%, at least about 60%, at least about 65% or at least about 70%. In an embodiment the decrease can be at least about 40%.

In one or more embodiments the hydrophobic vehicle foam or gel used in the method results in a decrease of at least about 55% the total number of acne vulgaris lesions four weeks after the end of the treatment In one or more embodiments the decrease is at least about 57%, or at least about 60%, or at least about 62%, or at least about 65%, or at least about 68%.

In one or more embodiments the hydrophobic gel or foam vehicle used in the method obtains a decrease of at least 45% in the total number lesions after nine weeks or less than nine weeks of treatment, when the composition is administered on average once daily. In one or more embodiments the decrease is at least about 50%, or at least about 55% at least about 60%, or at least about 65%. In an embodiment the decrease can be at least about 40%.

In one or more embodiments the hydrophobic gel or foam vehicle used in the method obtains a mean decrease of at least 40% in the total number lesions after six weeks or less than six weeks of treatment, when the composition is administered on average once daily. In one or more embodiments the mean decrease is at least about 45%, or at least about 50%, or at least about 55% at least about 60%.

In one or more embodiments the hydrophobic gel or foam vehicle used in the method obtains at least a 25% mean decrease in the total number lesion after three weeks or less than three weeks of treatment, when the vehicle is administered on average once daily. In one or more embodiments the decrease is at least about 30%, or at least about 35% at least about 42%, or at least about 45%, or at least about 50%. In one or more embodiments the hydrophobic gel or foam vehicle used in the method obtains at least a 40% mean decrease in the total number lesion after three weeks or less than three weeks of treatment, when the vehicle is administered on average once daily.

In one or more embodiments the hydrophobic gel or foam vehicle used in the method obtains a decrease of more than 50% of the non-inflammatory lesions in at least 70% of the subjects after 12 weeks of treatment. In one or more embodiments the mean decrease is at least about 50%, or at least about 60%, or at least about 70%, or at least about 80%, and the percent of subjects is at least about 25%, or at least about 30%, or at least about 35%, or at least about 40%, or at least about 45%, or at least about 50%, or at least about 55%, or at least about 60%, or at least about 65%, or at least about 72%, or at least about 75% at least about 80%. In an embodiment the decrease can be at least about 40%.

In one or more embodiments the hydrophobic gel or foam vehicle used in the method obtains a decrease of more than 50% of the inflammatory lesions in at least 50% of the subjects after 6 weeks of treatment. In one or more embodiments the mean decrease is at least about 50%, or at least about 55%, or at least about 60%, or at least about 70%, or at least about 80%, and the percent of subjects is at least about 25%, at least about 30%, at least about 35%, at least about 40%, or at least about 50%, or at least about 55%, or at least about 60%, or at least about 65%. In an embodiment the decrease can be at least about 40%.

In one or more embodiments the hydrophobic gel or foam vehicle used in the method obtains a decrease of more than 50% of the inflammatory lesions in at least 55% of the subjects after 12 weeks of treatment. In one or more embodiments the mean decrease is at least about 50%, or at least about 55%, or at least about 60%, or at least about 70%, or at least about 80%, and the percent of subjects is at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%. In an embodiment the decrease can be at least about 40%.

In one or more embodiments the hydrophobic gel or foam vehicle used in the method obtains a decrease of more than 50% of the inflammatory lesions in at least 50% of the subjects after 6 weeks of treatment. In one or more embodiments the mean decrease is at least about 50%, or at least about 55%, or at least about 60%, or at least about 70%, or at least about 80%, and the percent of subjects is at least about 10%, or at least about 15%, or at least about 20%, or at least about 25%, at least about 30%, at least about 35%, at least about 40%, or at least about 50%, or at least about 55%, or at least about 60%, or at least about 65%, or at least about 70%. In an embodiment the decrease can be at least about 40%.

In one or more embodiments the hydrophobic gel or foam vehicle used in the method obtains a decrease of more than 50% of the total number of lesions in at least 65% of the subjects after 12 weeks of treatment. In one or more embodiments the mean decrease is at least about 50%, or at least about 55%, or at least about 60%, or at least about 70%, or at least about 80%, and the percent of subjects is at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%. In an embodiment the decrease can be at least about 40%.

In one or more embodiments the hydrophobic gel or foam vehicle used in the method obtains a decrease of more than 50% of the total number of lesions is at least about 50%, or at least about 55%, or at least 60% of the subjects after 6 weeks of treatment. In one or more embodiments the mean decrease is at least about 60%, or at least about 70%, or at least about 80%, and the percent of subjects is at least about 20%, or at least about 25%, at least about 30%, at least about 35%, at least about 40%, or at least about 50%, or at least about 55%, or at least about 60%, or at least about 65%, or at least about 70%.

In one or more embodiments the decrease in inflammatory lesions or the decrease in non-inflammatory regions or the decrease in total lesions following application of the vehicle daily for about two weeks, or for about 3 weeks, or for about four week, or for about five weeks, or for about six weeks, or for about seven weeks, or for about eight weeks, or for about nine weeks, or for about ten weeks, or for about eleven weeks, or for about twelve weeks is between about 35% to about 65%, or between about 36% to about 63%, or between about 37% to about 62%, or between about 38% to about 61%, or between about 39% to about 60%, or between about 40% to about 59%, or between about 40% to about 57%, or between about 40% to about 57%, or between about 40% to about 55%.

In one or more embodiments the hydrophobic gel or foam vehicle used in the method the percent reduction in the count of inflammatory lesions or inflammatory lesions or total lesions reaches a steady state after 6 weeks of treatment.

In one or more embodiments the hydrophobic gel or foam vehicle used in the method obtains a percent reduction in the number of inflammatory lesion or inflammatory lesions or total lesions which continues to the follow-up visit e.g. at about four weeks after cessation of treatment.

In one or more embodiments the hydrophobic gel or foam vehicle used in the method results in a mean decrease of 30% or more than about 30% from baseline in the IGA score after twelve weeks of treatment.

In one or more embodiments the percentage of patients with IGA “almost clear” or “clear” receiving the hydrophobic gel or foam vehicle is at least 20% or more than about 20% after 6 weeks of treatment. In one or more embodiments the percentage of patients with IGA “almost clear” or “clear” who used the hydrophobic gel or foam, is at least 33% or more than about 33% four weeks after the end of treatment.

In one or more embodiments the percentage of patients receiving hydrophobic gel or foam vehicle with an IGA change of at least 2 units is at least 15% or more than about 15% after nine weeks of treatment.

In one or more embodiments at least about a third of patients receiving the hydrophobic gel or foam vehicle have an ‘excellent’ improvement and at least about 60% of subjects have an ‘excellent’ or ‘moderate’ improvement as assessed by the physician after twelve weeks of treatment. In one or more embodiments at least about 25% of subjects receiving the hydrophobic gel or foam vehicle evaluate their acne as “much better than prior to study”. In one or more embodiments at least about 75% receiving the hydrophobic gel or foam vehicle evaluate their acne as “slightly better than prior to study”.

In one or more embodiments the hydrophobic gel or foam vehicle used in the method comprises:

about 48% to about 51% by weight of soybean oil;
about 23% to about 25% by weight of coconut oil;
about 4% to about 6% by weight of cyclomethicone;
about 0.5% to about 6% by weight of light mineral oil;
about 3% to about 4% by weight of cetostearyl alcohol;
about 2% to about 4% by weight of stearic acid;
about 2% to about 3% by weight of myristyl alcohol;
about 1% to about 3% by weight of hydrogenated castor oil;
about 1% to about 3% by weight of beeswax;
about 1% to about 2% by weight of stearyl alcohol;
about 0.5% to about 1.5% by weight of behenyl alcohol;
about 0.2% to about 0.5% by weight of modified (fumed) silica; and
optionally a color agent and or a cosmetic agent.

In one or more embodiments the hydrophobic gel or foam vehicle used in the method further comprises trace amounts of an active pharmaceutical ingredient or in such amounts as to be considered effective as a homeopathic treatment. In one or embodiments the homeopathic formulation comprising the hydrophobic gel or foam vehicle comprises a tetracycline antibiotic. In one or more embodiments the tetracycline antibiotic is a minocycline. In an embodiment the tetracycline is between about 0.0005% and 0.005%. In an embodiment the tetracycline is between about 0.005% and 0.05%. In an embodiment the tetracycline is between about 0.05% and 0.5%. In one or more embodiments the tetracycline is about 0.005%, or about 0.006%, or about 0.007%, or about 0.008%, or about 0.009%, or about 0.01%, or about 0.02%, or about 0.03%, or about 0.04%, or about 0.05%.

In one or more embodiments the hydrophobic foam used in the method is formed from the hydrophobic gel composition further comprises about 3% to about 25% by weight of propellant based on the total weight of the hydrophobic gel vehicle.

In one or more embodiments the hydrophobic gel or foam vehicle used in the method further comprises a color agent or a cosmetic agent. In one or more embodiments the cosmetic agent is about 0.05% to about 5% by weight and the color agent is about 0.0001% to about 0.1% by weight.

In one or more embodiments the hydrophobic foam or gel vehicle used in the method is safe and well tolerated when the hydrophobic gel or foam vehicle is administered once daily for a period of at least six weeks.

In one or more embodiments the tolerability of the hydrophobic foam or gel vehicle used in the method is determined by skin irritation and wherein symptoms for assessing skin irritation are selected from a group consisting of pigmentation, erythema, dryness, peeling, and itching.

In one or more embodiments the hydrophobic foam composition or gel vehicle used in the method is safe, and well tolerated and has high rates of clinical responses when the hydrophobic gel or foam vehicle is administered once daily for at least two weeks.

In one or more embodiments the method comprises a step of administering which includes releasing the hydrophobic gel or foam vehicle from a container and applying it onto the target area by collapsing and/or spreading it on the target area using mild mechanical force such as a simple rub thereby resulting in the hydrophobic gel or foam vehicle collapsing and being absorbed onto the target area. In one or more embodiments method further comprises using a sterile applicator or prior to the steps of administering and/or collapsing and/or spreading, the hands of the person spreading are sterilized in order to avoid cross contamination.

In one or more embodiments the hydrophobic gel or foam vehicle used in the method is mostly absorbed or absorbed within at least 120 seconds or within about 100 seconds, or within about 80 seconds or within about 60 seconds or within about 40 seconds.

In one or more other embodiments the vehicle is administered twice daily. In one or more further embodiments the vehicle is administered thrice daily.

In an embodiment the acne is severe. In an embodiment the acne is of moderate severity. In an embodiment the acne is of mild severity. In an embodiment the acne is almost clear. In one or more embodiments the treatment reduces the acne from severe to moderate severity or to mild severity or to almost clear or to clear. In one or more embodiments the treatment reduces the acne from moderate severity to mild severity or to almost clear or to clear. In one or more embodiments the treatment reduces the acne from mild severity to almost clear or to clear. In an embodiment the treatment reduces the acne from almost clear to clear.

In an embodiment the subject is under the age of 25 years old. In one embodiment the subject is under the age of thirty years old.

In one embodiment the subject the subject is under the age of forty-six years old and is a pregnant or breastfeeding female.

In one or more embodiments the hydrophobic gel or foam vehicle used in the method has a shelf life of at least two years at ambient temperature.

In one or more embodiments there is provided a method for protecting, ameliorating, retarding, arresting, or reversing the progression of a disorder in a mammalian subject in need thereof, the disorder selected from the group consisting of acne, acne related symptoms, a tetracycline antibiotic responsive acne related disorder, a tetracycline antibiotic responsive skin disorder, skin disorder caused by a bacteria, a tetracycline antibiotic responsive disorder, a sebaceous gland disorder, P. acne bacteria associated disorders and other superficial infections, including skin infections, the method comprising topically applying to the skin of the subject a hydrophobic foam or gel vehicle at least alternate days or once a day for at least six weeks, thereby protecting, ameliorating, retarding, arresting, or reversing the progression of the disorder in the subject.

In one or more embodiments there is provided a method for retarding, arresting, or reversing the progression of a disorder wherein the hydrophobic gel or foam vehicle comprises:

about 60% to about 99% by weight of at least one hydrophobic solvent;

at least one viscosity-modifying agent selected from the group consisting of a fatty alcohol, a fatty acid, and a wax; and

a color agent or cosmetic agent.

In an embodiment of the invention is the use of a gel or foam vehicle as described before, wherein (a) at least one hydrophobic solvent is selected form a group consisting of light mineral oil, cyclomethicone, coconut oil, soybean oil, or a mixture of any two or more thereof (b) at least one viscosity-modifying agent is selected form a group consisting of cetostearyl alcohol or stearyl alcohol or myristyl alcohol or cetostearyl alcohol or behenyl alcohol or stearic acid or beeswax or hydrogenated castor oil or a mixture of any two or more thereof together with a propellant for the treatment of human skin especially for the treatment of acne, acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections.

In an embodiment of the invention the gel or foam vehicle further comprises at least one preservative and/or at least one color agent.

In an embodiment of the invention the gel or foam vehicle as described before comprises Aerosil (SiO₂). In an embodiment of the invention the gel or foam vehicle as described before comprises quinoline yellow WS (D&C yellow 10) and/or quinoline yellow SS (D&C yellow 11) as a color agent.

In an embodiment of the invention is the use of a gel vehicle as described before, containing (a) a mixture of about 5.49 weight percent light mineral oil, about 5 weight percent cyclomethicone, about 23.60 weight percent coconut oil, about 50 weight percent soybean oil and about 2 weight percent hydrogenated castor oil, (b) a mixture of about 3.5 weight percent cetostearyl alcohol, about 2.5 weight percent myristyl alcohol, about 1.5 weight percent stearyl alcohol, about 1.1 weight percent behenyl alcohol, about 3 weight percent stearic acid and about 2 weight percent beeswax (c) about 0.25 weight percent aerosol (SiO₂), (d) about 0.05 quinoline yellow WS (D&C yellow 10) and about 0.001 quinoline yellow SS (D&C yellow 11) which together with a propellant forms a foam for the treatment of human skin including e.g. the treatment one or more of acne, acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections.

In an embodiment the disorder is selected from the group consisting of a wound, a chronic wound, a burn, impetigo, acne, rosacea, an inflammation, an ulcer, and a skin disease caused by a bacteria. In an embodiment the disorder is a wound. In an embodiment the disorder is a chronic wound. In an embodiment the disorder is a burn. In an embodiment the disorder is impetigo. In an embodiment the disorder is acne. In an embodiment the disorder is rosacea. In an embodiment the disorder is an inflammation. In an embodiment the disorder is an ulcer. In an embodiment the disorder is a skin disease caused by a bacteria. In an embodiment the disorder is a skin disease caused by a fungus. In an embodiment the disorder is a skin disease caused by a virus.

As propellant a compound may be used, which is a gas at room temperature under normal pressure and which may be liquified at increased pressure at room temperature. Useful propellants are butane, propane, isobutene, dimethylether, fluorocarbon gases or mixtures thereof. Any compatible propellant may be used. In one or more embodiments, the propellant is a gas at room temperature under normal pressure and which may be liquefied at increased pressure at room temperature. Examples of propellants include, without limitation, hydrocarbon propellants such as butane, propane, isobutane, dimethyl ether, fluorocarbons such as 1,1,1,2 tetrafluorethane (Dymel 134), and 1,1,1,2,3,3,3 heptafluoropropane (Dymel 227), and mixtures thereof. In one or more embodiments, a hydrocarbon mixture AP-70 (a mixture of about 30% w/w butane, 20% w/w isobutane and 50% w/w propane) is used. In one or more embodiments a gas propellant like nitrogen or carbon dioxide is used.

The vehicle according to the invention is manufactured according to the methods described in the art which are known to a pharmaceutical expert. It is usually packed in a canister with an outlet valve. Possible canisters and valves are likewise described in the art and do not need to be explained in this document.

In some embodiments, the vehicle is substantially alcohol-free, i.e., free of short chain alcohols (with 1-4 carbon atoms chain length).

One known disadvantage of state of the art compositions is the low solubility of the pharmaceutically active agents. It is therefore an advantage of the vehicle according to the present invention that there is no need to dissolve any pharmaceutically active agents.

One known disadvantage of state of the art compositions is achieving chemical stability of the pharmaceutically active agents within the composition. It is therefore an advantage of the vehicle according to the present invention that there is no need to stabilize any pharmaceutically active agents.

In Phase II clinical trials it has been shown that the vehicle according to the description provided herein has beneficial properties, especially in the treatment of acne. It was very surprising to note that this therapeutic effect was achieved without application of any pharmaceutically active agents and despite having a high oil content. A number of further medical conditions can be treated with the vehicle according to the present invention such as acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections, wound, a chronic wound, a burn, impetigo, acne, rosacea, an inflammation, an ulcer, and a skin disease caused by a bacteria. Furthermore, the vehicle described herein may be used for a prophylactic treatment of the human skin (e.g. in patients with a known tendency to develop such disease).

The vehicle according to the description provided herein may also be used for a cosmetic treatment of the human skin.

It is therefore another aspect of the invention to provide a method of treating human skin disorders such as acne, acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections, wound, a chronic wound, a burn, impetigo, acne, rosacea, an inflammation, an ulcer, and a skin disease caused by a bacteria by topical application of a foam or gel vehicle as described herein to a patient in need thereof.

It is a further aspect of the invention to provide a method of prophylactic treatment of human skin, especially for humans with a known tendency to develop skin disorders such as acne, acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections, wound, a chronic wound, a burn, impetigo, acne, rosacea, an inflammation, an ulcer, and a skin disease caused by a bacteria by topical application of a gel or foam vehicle as described herein to such human.

It is a still further aspect of the invention to provide a method of cosmetic treatment of human skin by topical application of a gel or foam vehicle as described herein to a human.

For all of these applications described herein (therapeutic, prophylactic or cosmetic) the following vehicle essentially free of active pharmaceutical agents packed in a canister with an outlet valve has been found to be most useful.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in the definition section prevail unless stated otherwise.

### BRIEF DESCRIPTION OF THE DRAWINGS

The invention is described with reference to the drawings, which are presented for the purpose of illustration only and is not intended to be limiting of the invention.

FIG. 1 provides a chart summarizing the mean inflammatory lesion count in the ITT population by visit and by study group.

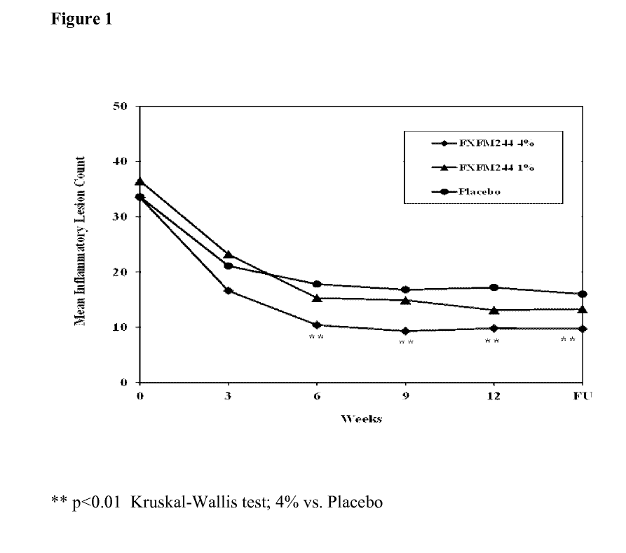

FIG. 2 provides a chart summarizing the mean reduction in the number of inflammatory lesion in the ITT population by visit and by study group.

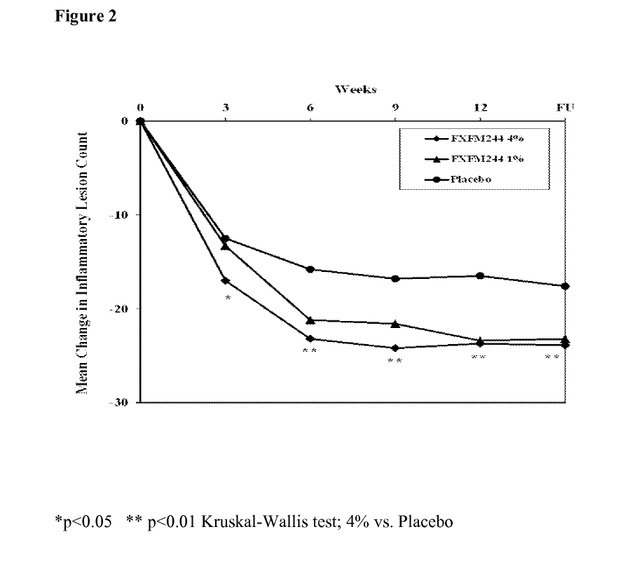

FIG. 3 provides a chart summarizing the mean percent reduction of the number of inflammatory lesions in the ITT population by visit and by study group.

FIG. 4 provides a chart summarizing the percent of subjects who had a decrease of more than 60%, in inflammatory lesions count in the ITT population by visit and by study group.

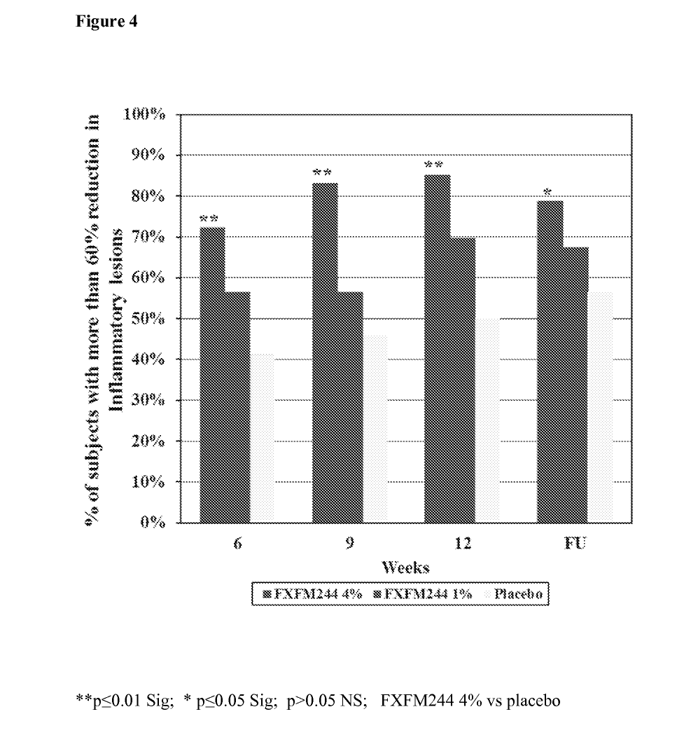

FIG. 5 provides a chart summarizing the mean percent reduction of the number of non-inflammatory lesions in the ITT population by visit and by study group.

FIG. 6 provides a chart summarizing the mean percent reduction of the total number of lesions in the ITT population by visit and by study group.

FIG. 7 provides a chart summarizing the mean IGA score in the ITT population by visit and by study group.

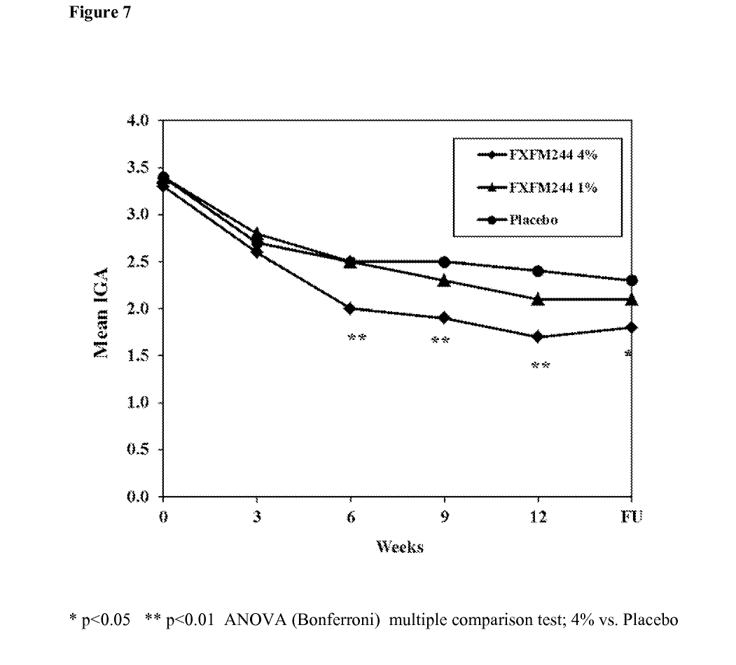

FIG. 8 provides a chart summarizing the percent of subjects with mean IGA score of less than 2 in the ITT population by visit and by study group.

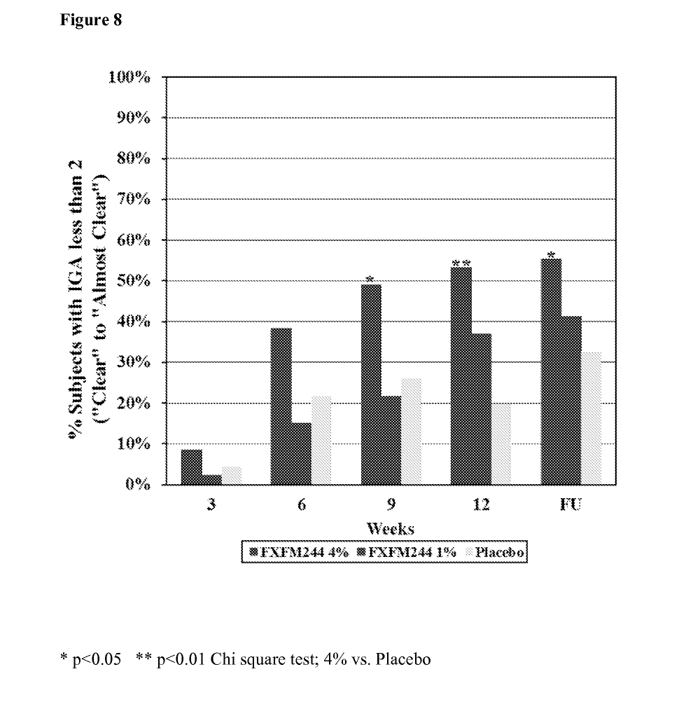

### DETAILED DESCRIPTION

In one or more embodiments there is provided herein, a method for treating a disorder of the skin or a mucosal surface, especially when the disorder is a disorder of a sebaceous gland. In one or more embodiments there is provided herein, a method for treating a disorder including acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infection, including skin infections. In one or more embodiments the disorder involves inflammation. The method includes administering topically to a surface having the disorder a hydrophobic vehicle in a form of a foam or gel comprising about 60% to about 99% by weight of at least one hydrophobic solvent and at least one viscosity-modifying agent selected from the group consisting of a fatty alcohol, a fatty acid and a wax. In one or more embodiments the placebo had a substantial and unexpected positive effect.

In one or more embodiments, a hydrophobic foamable vehicle (e.g. foam or gel) provided herein comprises:

a) about 78% to about 99% by weight of at least one hydrophobic solvent;
b) about 1% to about 22% by weight of at least one viscosity modifying agent.

In one or more embodiments, a hydrophobic vehicle in a form of a foam or gel provided herein comprises:

a) about 78% to about 90% by weight of at least one hydrophobic solvent;
b) about 10 to about 22% by weight of at least one viscosity modifying agent.

In one or more embodiments, a hydrophobic vehicle in a form of a foam or gel provided herein comprises:

a) about 75% to about 90% by weight of at least one hydrophobic solvent;
b) about 10 to about 22% by weight of at least one viscosity modifying agent; and
c) about 0.5% to about 3% of a color agent and/or a cosmetic agent.

In one or more embodiments, a hydrophobic vehicle in a form of a foam or gel provided herein comprises:

a) about 72% to about 88% by weight of at least one hydrophobic solvent;
b) about 10 to about 22% by weight of at least one viscosity modifying agent; and
c) about 2% to about 6% of a color agent and/or a cosmetic agent.

According to one or more embodiments there are provided substantially surfactant-free oleaginous vehicle, for use in treatment of acne, acne related symptoms, a tetracycline antibiotic responsive acne related disorder, a tetracycline antibiotic responsive skin disorder, skin disorder caused by a bacteria, a tetracycline antibiotic responsive disorder, a sebaceous gland disorder, P. acne bacteria associated disorders and other superficial infections, including skin infections. In one or more embodiments the vehicle is used for the treatment of rosacea. In one or more embodiments the vehicle is used for the treatment of impetigo. In one or more embodiments the composition vehicle to reduce oxidative stress and/or inflammation in skin pathologies. In one or more embodiments the vehicle is effective where the condition is accompanied by apoptotic cell death.

### DEFINITIONS

All % values are provided on a weight (w/w) basis.

By the term “about” herein it is meant that a figure or range of figures can vary plus or minus up to 10%. So in this embodiment if a figure of “about 1” is provided then the amount can be up to 1.1 or from 0.9. As will be appreciated by one of the art there is some reasonable flexibility in formulating compositions such that where one or more ingredients are varied successful formulations may still be made even if an amount falls slightly outside the range. Therefore, to allow for this possibility amounts are qualified by about. In one or more other embodiments the figures may be read without the prefix about.

The term “pharmaceutically active compounds” or “pharmaceutically active agents or ingredients” refers to compounds with proven pharmaceutical activity demonstrated in clinical trials and approved as a drug by the European Medicines Agency (EMEA) or the US Food and Drug Administration (FDA).

The term “essentially free of pharmaceutically active compounds” or “essentially free of pharmaceutically active agents or ingredients” means that no “pharmaceutically active compound” or “pharmaceutically active agent or ingredient” has been intended to be added to the composition. The total amount of pharmaceutically active agents as a result of unintended contamination is therefore well below 0.05%, preferably below 0.01%. Most preferred is a composition in which no amount of any pharmaceutical active agent can be detected with standard analytical methods used in pharmaceutical technology.

The term “thixotropic,” as used herein, means that the formulation shows a decrease in viscosity upon application of shear force. The structure of the formulation breaks down, leading to a reduction in viscosity. When the formulation is standing without shear force, this decrease in viscosity is recovered over time.

It should be noted that the term “gel” means a jelly-like material that can have properties ranging from soft and fluid to hard and tough. Gels may be in liquid, semi-liquid, semi-solid or solid state. Solid gels are defined as a substantially diluted crosslinked system, which exhibits no flow when in the steady-state. By weight, gels are mostly liquid, yet they behave like semi-solids due to a three-dimensional crosslinked network of a solidifying, gelling or thickening agent within the liquid. It is the crosslinks within the fluid that give a gel its structure (hardness) and contribute to stickiness (tack). Depending on the amounts of gelling agent in a formulation, the gel may be semi-solid with some limited flowability, such that when the semi-solid gel is placed in a tube and is inclined horizontally from a vertical position it will slowly flow from the vertical towards the horizontal or it may be a liquid gel where the amount of gelling agent or gelling effect is lower such that the gel structure or connections are weaker or loose so that when placed in a tube and tilted from a vertical position to the horizontal the gel readily flows and adapts to the horizontal position. The rheological properties of gels at different surface temperatures can influence the release and bioahsorption of drugs therefrom.

In some embodiments, formulations comprising a hydrophobic oil and viscosity-modifying agents demonstrated increased viscosity of such oil.

In one or more embodiments, the gel is stable and it retains its viscosity upon dispensing from a container, such as a tube, yet, it liquefies and spreads easily upon application of shear force, which can be mild, such as a simple rub. Further, while the gel is oily, it absorbs into the site of application, such as the skin or mucosa membrane, and after minutes the surface does not appear and/or feel significantly oily or greasy.

The term “liquid gel” refers inter alia to the formulation after propellant is added (which prior to adding the propellant is a gel) or where the gel is loose or fluid or such that when subjected to gravity will pour or become liquid.

The term “vehicle” is explained in the Background.

The term “waterless” or “water free” as used herein, means that the vehicle contains no, or essentially no, free or unassociated or absorbed water. Similarly, “substantially water free” or “substantially waterless” vehicle contain at most incidental or trace amounts of water. In one or more embodiments, “substantially waterless” or “substantially water free” means the vehicle contains about or less than 1%, about or less than 0.8%; about or less than 0.6%; about or less than 0.4%; about or less than 0.2%; about or less than 0.1%, about or less than 0.5%, about or less than 0.1% about or less than 0.05%, about or less than 0.01%, less than about 0.001% by weight, or 0% by weight).

By the term “single phase” herein it is meant that after addition of propellant to the vehicle, the liquid components of the foamable vehicle are fully miscible, and the solid components, if any, are either dissolved or homogeneously suspended in the vehicle so that only one phase is visible.

By the term “substantially a single phase” is meant that the vehicle after addition of propellant is primarily or essentially a single phase as explained above, but may also have present a small amount of material which is capable of forming or may form a separate phase amounting to less than about 5% of the vehicle after the addition of propellant, In an embodiment it is less than about 3%, and in another embodiment is less than about 1%. The term “unstable active agent” as used herein, means an active agent which is oxidized and/or degraded within less than a day, and in some cases, in less than an hour upon exposure to air, light, skin, or water or a pharmaceutical excipient under ambient conditions.

It should be noted that the term “surfactant” or “emulsifier” in the context herein refers to stand alone surfactants used to reduce surface tension between two substances or phases, which arc also capable of stabilizing an emulsion of water and oil. Reduction of surface tension can be significant in foam technology in relation to the ability to create small stable bubbles. This is as opposed to the term surfactant which has often been loosely used in the art to include substances which do not function effectively as standalone surfactants to reduce surface tension between two substances or phases and which are also capable of stabilizing an emulsion of water and oil. For example, a surfactant as provided herein, does not include fatty acids, does not include fatty alcohols and does not include propoxylated lanolin oil derivatives. In the context of the present invention fatty acids and fatty alcohols are defined as foam adjuvants. Similarly, propoxylated lanolin oil derivatives in the context herein are defined as emollients.

“Standard surfactant” or “customary surfactant” or “stand alone surfactant” refers to customary non-ionic, anionic, cationic, zwitterionic, amphoteric and amphiphilic surfactants. Many standard surfactants are derivatives of fatty alcohols or fatty acids, such as ethers or esters formed from such fatty alcohols or fatty acids with hydrophilic moieties, such as polyethylene glycol (PEG). However, a native (non-derivatized) fatty alcohol or fatty acid, as well as waxes are not regarded as a standard surfactant.

The term “co-surfactant” as used herein, means a molecule which on its own is not able to form and stabilize satisfactorily an oil-in-water emulsion but when used in combination with a surfactant as defined herein the co-surfactant has properties which can allow it to help a surfactant create an emulsion and can boost the stabilizing power or effect of the surfactant. Examples include a fatty alcohol, such as cetyl alcohol, or a fatty acid, such as stearic acid. Cetyl alcohol is a waxy hydrophobic substance that can be emulsified with water using a surfactant. Some substances may have more than one function and for example, fatty alcohols can in some formulations act as a co-solvent. In certain circumstances, a co-surfactant can itself be converted into a surfactant or soap by, for example, adding a base, such as, triethanolamine to a fatty acid like stearic acid.

The term “viscosity modifying agent” in the context of the present disclosure is an agent which, when added to a hydrophobic oil, facilitates the creation of a hydrophobic breakable vehicle in the form of a breakable gel or breakable foam. In one or more embodiments, the viscosity modifying agent is selected from a group consisting of a fatty alcohol, a fatty acid and/or a wax. The term “viscosity modifying agent” is also known as a “foamer complex”.

The term “breakable” refers to a unique property of the gel or the foam wherein the gel or foam is stable upon dispensing from a container, yet breaks and spreads easily upon application of shear or mechanical force, which can be mild such as a simple rub.

It should be noted that the term a “polyol”, as used herein, is an organic substance that contains at least two hydroxy groups in its molecular structure.

The term “water activity” as used herein, represents the hygroscopic nature of a substance, or the tendency of a substance to absorb water from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Every microorganism has a limiting Aw, below which it will not grow; e.g., for Streptococci, Klebsiella spp, Escherichia coli, Clostridium perfringens, and Pseudomonas spp, the Aw value is 0.95. Staphylococcus aureus is most resistant and can proliferate with an Aw as low as 0.86, and fungi can survive at an Aw of at least 0.7. In one or more embodiments, the concentration of the hydrophobic solvent, and/or second rheology modulator in the composition is selected to provide an Aw value selected from the ranges between or of (1) about 0.8 and about 0.9; (2) about 0.7 and about 0.8; and (3) less than about 0.7. Delivering the formulation in a pressurized package does not allow for humidity to be absorbed by the preparation, and therefore, the water free character of the composition is not altered.

In one embodiment, no preservative is needed because the formulation is a waterless hydrophobic solvent or oil-based formulation having an Aw (water activity) value of less than 0.9, or less than about 0.8, or less than about 0.7, or less than about 0.6, and preferably less than about 0.5 which is below the level of microbial proliferation.

The identification of a “solvent,” as used herein, is not intended to characterize the solubilization capabilities of the solvent for any specific active agent or any other component of a gel or a foamable composition. Rather, such information is provided to aid in the identification of materials suitable for use as a part in the gel or a foamable vehicle described herein.

It should be noted that the term “a method of treating a disease or a disorder” as provided throughout the specification is interchangeable with the term “use of the composition as a medicament for treatment of a disease”. It should be noted the term a disease is used interchangeably with the term disorder.

It should be noted that the term “substantially free of” an ingredient as provided throughout the specification is intended to mean that the vehicle comprises less than about 0.5% by weight (e.g., less than about 0.2% by weight, less than about 0.1% by weight, less than about 0.05% by weight, less than about 0.01% by weight, less than about 0.001% by weight, or 0% by weight) of an ingredient.

The term “surfactant free” or emulsifier free” or “non-surfactant” composition means compositions which comprise no or negligible levels of surface active agents. Where a formulation includes insignificant or de minimis amounts of surface active agents it is considered to be essentially surfactant free.

The term “substantially surfactant-free” relates to a vehicle wherein the ratio between the viscosity-modifying agent and the surfactant is between 10:1 or 5:1; or between 20:1 and 10:1 or between 100:1 and 20:1. In additional embodiments, the term relates to a vehicle that contains a total of less than about 0.4% of a surfactant selected from the group consisting of customary non-ionic, anionic, cationic, zwitterionic, amphoteric and ampholytic surfactants.

Preferably, the composition comprises less than about 0.2% by weight of a standard surfactant or less than about 0.1%; or less than 0.05%.

By de minimis is meant so minor as to merit disregard.

The term “hydrophobic gel vehicle” or “hydrophobic foam vehicle” or “hydrophobic composition” is intended to mean that the vehicle has a low solubility in water. In an embodiment, 100 to 1000 parts of water are needed to dissolve or render miscible 1 part of vehicle. In an embodiment, 1000 to 10,000 parts of water are needed to dissolve or render miscible 1 part of vehicle. In an embodiment, more than 10,000 parts of water are needed to dissolve or render miscible 1 part of vehicle.

By “regular basis” is meant a repeated or repeatable interval of time which can be by way of illustration, a part of a day, daily, alternate daily, twice weekly, weekly, fortnightly, monthly or some other repeated or repeatable interval for an appropriate period of time wherein a dose is to be applied. In this connection the repeat applications will be according to the needs of the subject and the disease or disorder. In some circumstances as little as three repeat doses may be required in other cases, between 3 and 14, in other cases between 14 and 28, in other cases between 28 and 50, in other cases between 50 and 75, in other cases between 75 and 100 and in other cases such as where prolonged treatment or a long period of maintenance dosing is needed as many as one two or three hundred or more repeat doses may be needed.

The term clinical response to treatment, (clinical success or clinical failure) in the context of acne treatment is derived from an efficacy evaluation.

The term total lesion count relates to the sum of inflammatory lesions and non-inflammatory lesions.

The terms “high rates of clinical response” or “high efficacy” or “substantial decrease” in the context herein can relate to a reduction of about 45% or more in lesion count (inflammatory, non-inflammatory, total lesion); or to where subjects met a success criterion of “clear” or “almost clear”; or to an “improvement of 2 grades from the baseline; or to where subjects receive an excellent score according to Investigator's Global Improvement Assessment; or to where patients receive excellent in Patient's Global Improvement Assessment.

The term safe in the context herein means having no or essentially no adverse events or no serious adverse events. Safety was evaluated by assessing vital signs, physical examinations, and the incidence and severity of AEs

The term adverse event in the context of the clinical study means having any untoward sign (including an abnormal vital sign, physical exam finding), symptom, or disease temporally associated with the use of a medical product whether or not considered as related to the medical product. A new condition or the worsening of a pre-existing condition is considered as an adverse event. All abnormal findings or diagnostic procedures (vital signs, physical exam, pregnancy etc) considered to be clinically significant (CS) are recorded as adverse events.

The term serious adverse event in the context herein can relate to any adverse drug event (experience) occurring at any dose that results in any of the following outcomes: death, a life-threatening adverse drug experience, inpatient hospitalization or prolongation of existing hospitalization (for >24 hours, a persistent or significant incapacity or substantial disruption of the ability to conduct normal life functions, a congenital anomaly/birth defect, an important Medical Events (IME) that may not result in death, be life threatening, or require hospitalization may be considered a serious adverse drug experience when, based upon medical judgment, they may jeopardize the patient or subject and may require medical or surgical intervention to prevent one of the outcomes listed in this definition.

The term tolerable or enhanced tolerability in the context herein means having no or essentially no skin irritation symptoms such as pigmentation, erythema, dryness, peeling and itching or alternatively when such symptoms arise they are mild and disappear without interrupting treatment.

By “essentially no” in the context of tolerability includes insignificant or de minimis occurrences of skin irritation events manifested in symptoms such as pigmentation, erythema, dryness, peeling and itching mild transient events connected with the application of vehicle.

By “essentially no” in the context of safety includes insignificant or de minimis occurrences of systemic or serious adverse events or adverse events connected with the application of vehicle.

The clinical response was determined at each study visit inter alia by a lesion count (inflammatory/non inflammatory and total), by % change in lesion count, by Investigator global assessment, by improvement assessment (by the Investigator) and improvement assessment (by the patient). Photographs were also used to assess the clinical improvement.

The term clinical failure is defined as insufficient improvement or deterioration (i.e. an increase or no change in the number of lesions).

By “on average” with reference to dosage regimes it is intended to reflect and/or take into account human nature and that a subject may forget to apply a dose or not strictly adhere to the regime, such that even if a subject forgets a dose or does not strictly adhere to the regime it will still be considered as if the regime has been applied. For example, if a subject misses an occasional dose but does not make it up or alternatively if having missed a dose applies a compensatory dose on a different day it is still counted as having complied with the dosage regime

An acne related disorder is any disorder which may occur in parallel with acne or be a contributing factor to the outbreak of acne or may resemble acne. Perioral dermatitis is an erythematous, papulopustular facial eruption that resembles acne and/or rosacea but typically starts around the mouth. Rosacea (acne rosacea) is a chronic inflammatory disorder characterized by facial flushing, telangiectasias, erythema, papules, pustules, and in severe cases, rhinophyma.

Acne related symptoms include, papules, pustules, blackheads, whiteheads or milia, nodules and cysts.

Acne conglobata is the most severe form: of acne vulgaris, affecting men more than women. Patients have abscesses, draining sinuses, fistulated comedones, and keloidal and atrophic scars. Acne fulminans is acute, febrile, ulcerative acne, characterized by the sudden appearance of confluent abscesses leading to hemorrhagic necrosis. Leukocytosis and joint pain and swelling may also be present. Pyoderma faciale (also called rosacea fulminans) occurs suddenly on the midface of young women. It may be analogous to acne fulminans. The eruption consists of erythematous plaques and pustules.

In non-inflammatory acne, symptoms include microcomedones, blackheads, milia and closed comedones, bumps or bumpiness across the skin's surface, or an uneven skin texture, rough skin feel, non-inflamed acne blemishes, acne cosmetica, smoker's acne. Inflamed acne is characterized by redness and inflammation. Those with inflamed acne will have microcomedones, blackheads, and milia, as well as papules, pustules, and possibly nodules and cysts. Symptoms also include redness, swelling, and irritation of the skin, along with possible crusting, oozing, or scabbing of the lesions. Inflamed acne ranges in acuity from very mild to extremely severe and from only the occasional pustule to angry-looking cysts, skin damage and scarring and pigmentation. These symptoms can typically be seen on the face, neck, chest, shoulders, upper arms, back and less commonly on the torso, arms, and legs. Discolored, darkened, or reddened spots or blotches are common on acne prone skin. These problems can persist even after breakouts have fully healed. Post-inflammatory hyperpigmentation (PIH) describes discolored spots (macules) left behind after an acne lesion has healed. Scars include ice pick scars, rolling scars, atrophic (depressed or pitted) scars, boxcar scars, hypertrophic (raised) scars and keloids.

In one or more embodiments, the vehicle may be effective in treatment of one or more of bacterial infections, cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma, disorders of hair follicles and sebaceous glands, acne, rosacea, perioral dermatitis, hypertrichosis (hirsutism), alopecia, including male pattern baldness, alopecia greata, alopecia universalis and alopecia totalis, pseudofolliculitis barbae, and keratinous cyst. For example, rosacea involves papules and pustules, as well as erythema, telangiectasia, and redness.

It should be noted that hydrophobic vehicle disclosed herein can be applied to the target site as a gel or a semi-solid gel or foam. In certain other embodiments, it can be applied as a liquid gel or as a collapsed foam. In one or more embodiments, the vehicle is thixotropic. In one or more embodiments, the gel formulation subjected to constant shear rate shows a reduction in viscosity with time. In one or more further embodiments, after the material is allowed to rest for a period of time, the viscosity increases again. In one or more embodiments, there is provided prior to adding propellant a solid or semi-solid composition or gel. In one or more embodiments, the composition or gel is a liquid. In one or more embodiments the propellant is miscible with and dilutes the vehicle.

Upon packaging of the foamable vehicle in an aerosol container and adding a propellant, a shakable and homogenous foamable vehicle is prepared, which upon dispensing forms a breakable foam with good to excellent quality. The resulting foam is pharmaceutically equivalent to the respective gel (prior to adding the propellant), since immediately upon dispensing of the foam the propellant evaporates and the composition upon collapsing is similar or identical to that of the gel. This is an important pragmatic advantage, because many drug development activities, including expensive and lengthy toxicology studies with numerous animals and clinical trials with thousands of patients can be saved by conducting such studies once for either the gel or foam presentation instead of twice (for each presentation).

Application can be, hourly, twelve hourly (e.g., twice daily), daily, alternate-day or intermittent, according to the condition of the patient. For reasons of compliance, less frequent applications, where possible, are preferable, e.g., daily single applications. In certain cases, where prolonged or long term treatment is required, an initial dose is provided followed by a gradual reduction to a lower maintenance dose, which can be increased if further outbreaks occur.

The vehicle may further include a color agent and/or a cosmetic agent. In one or more embodiments, the color agent and/or cosmetic agent is about 18%, or about 17.5%, or about 16.5%, or about 15.5%, or about 14.5%, or about 13.5%, or about 12.5%, or about 11.5%, or about 10.5%, or about 9.5%, or about 8.5%, or about 7.5%, or about 6.5%, or about 5.5%, or about 4.5%, or about 3.5%, or about 2.5%, or about 1.5%, or about 17%, or about 16%, or about 15%, or about 14%, or about 13%, or about 12%, or about 11%, or about 10%, or about 9%, or about 8%, or about 7%, or about 6%, or about 5%, or about 4%, or about 3, % or about 2%, or about 1%, or about 0.75%, or about 0.5%, or about 0.25%, or about 0.2% by weight of the vehicle.

In one or more embodiments, the color agent comprises a color additive, a dye, a colorant or an indicator. In one or more embodiments the color additive is D&C Yellow No. 10. In one or more embodiments the color additive is D&C Yellow #11.

D&C Yellow No. 10 is a mixture of the sodium salts of the mono- and disulfonic acids of 2-(2-quinolinyl)-1H-indene-1,3(2H)-dione consisting principally of the sodium salts of 2-(2,3-dihydro-1,3-dioxo-1H-indene-2-yl)-6-quinolinesulfonic acid and 2-(2,3-dihydro-1,3-dioxo-1H-indene-2-yl)-8-quinolinesulfonic acid with lesser amounts of the disodium salts of the disulfonic acids of 2-(2-quinolinyl)-1H-indene-1,3(2H)-dione. It is manufactured by condensing quinaldine with phthalic anhydride to give the unsulfonated dye, which is then sulfonated with oleum. The color additive D&C Yellow No. 11 is principally 2-(2-quinolyl)-1,3-indandione.

In one or more embodiments, such a vehicle is presented as a breakable gel, which breaks down with mild mechanical force.

In one or more embodiments, the hydrophobic vehicle when packaged in an aerosol container to which is added a liquefied or compressed gas propellant the composition provides upon release from the container a breakable foam of at least good quality that breaks easily upon application of mechanical force.

In one or more embodiments, the vehicle is a foamable composition that is thermally stable at skin temperature.

In one or more embodiments, when the above vehicle is filled into an aerosol can or canister and pressurized with a propellant a foamable vehicle is produced.

In one or more embodiments the method comprises:

1. expelling a foamable vehicle from a canister;

2. applying the resultant foam to an area of skin or mucosa;

3. collapsing the foam.

In one or more embodiments the method comprises:

1. expelling a foamable vehicle from a canister;
2. applying the resultant foam to an area of skin or mucosa;
3. spreading the foam.

In one or more embodiments the spread or collapsed foam is absorbed into the skin or mucosa. In one or more embodiments the absorption is rapid. In one or more embodiments it is mostly absorbed or within about 30 seconds, or within about 20 seconds, or within about 15 seconds, or within about 10 seconds, or within about 5 seconds, or within about 3 seconds or within about a second. In one or more embodiments absorption is within about 30 seconds, or within about 20 seconds, or within about 15 seconds or within about 10 seconds, or within about 5 seconds, or within about 3 seconds or within about a second.

In one or more embodiments, the at least one hydrophobic solvent comprises or is selected from the group consisting of a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, liquid paraffin, an isoparaffin, a polyalphaolefin, a polyolefin, polyisobutylene, a synthetic isoalkane, isohexadecane, isododecane, alkyl benzoate, alkyl octanoate, C₁₂-C₁₅ alkyl benzoate, C₁₂-C₁₅ alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, avocado oil, apricot stone oil, barley oil, borage seed oil, calendula oil, canelle nut tree oil, canola oil, caprylic/capric triglyceride castor oil, coconut oil, corn oil, cotton oil, cottonseed oil, evening primrose oil, flaxseed oil, groundnut oil, hazelnut oil, glycereth triacetate, glycerol triheptanoate, glyceryl trioctanoate, glyceryl triundecanoate, hempseed oil, jojoba oil, lucerne oil, maize germ oil, marrow oil, millet oil, neopentylglycol dicaprylate/dicaprate, olive oil, palm oil, passionflower oil, pentaerythrityl tetrastearate, poppy oil, propylene glycol ricinoleate, rapeseed oil, rye oil, safflower oil, sesame oil, shea butter, soya oil, soybean oil, sweet almond oil, sunflower oil, sysymbrium oil, syzigium aromaticum oil, tea tree oil, walnut oil, wheat germ glycerides, wheat germ oil, PPG-2 butyl ether, PPG-4 butyl ether, PPG-5 butyl ether, PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether, PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, herring oil, cod-liver oil, salmon oil, cyclomethicone, a dimethyl polysiloxane, dimethicone, an epoxy-modified silicone oil, a fatty acid-modified silicone oil, a fluoro group-modified silicone oil, a methylphenylpolysiloxane, phenyl trimethicone and a polyether group-modified silicone oil. In some embodiments, the hydrophobic solvent comprises or is selected from the group consisting of soybean oil, a coconut oil, a cyclomethicone, a light mineral oil, and mixtures thereof.

In one or more embodiments, the hydrophobic solvent is at a concentration of about 75% to about 90% by weight. In one or more embodiments, the hydrophobic solvent is at a concentration of at least about 40% by weight, at least about 50% by weight, at least about 60% by weight, at least about 70% by weight, at least about 90% by weight. In some embodiments, the hydrophobic solvent is at a concentration of less than about 90% by weight, less than about 80% by weight, less than about 70% by weight, less than about 60% by weight, less than about 50% by weight.

In one or more embodiments, the viscosity-modifying agent is at a concentration of about 0.1% to about 22%, about 0.4 to about 18%, about 0.5% to 16%, about 0.6% to 14%, about 0.7% to 13%, about 0.8 to about 12%, about 0.9% to about 11%, about 1% to about 10%, about 10% to about 22% by weight. In one or more embodiments, the viscosity-modifying agent is a fatty alcohol having at least 12 carbon atoms in its carbon backbone. In one or more embodiments, the viscosity-modifying agent is a fatty acid having at least 12 carbon atoms in its carbon backbone.

In one or more embodiments, the viscosity-modifying agent is at a concentration of about 9.5%, or about 8.5%, or about 7.5%, or about 6.5%, or about 5.5%, or about 4.5%, or about 3.5%, or about 2.5%, or about 1.5%, about 7%, or about 6%, or about 5%, or about 4%, or about 3%, or about 2%, or about 1%, or about 0.5%, or about 1.9%, or about 1.8%, or about 1.7%, or about 1.6%, or about 1.55, or about 1.4%, or about 1.3%, or about 1.2%, or about 1.1%, or about 0.9%, or about 0.8%, or about 0.7%, or about 0.6%, or about 0.5%, by weight of the composition or less than any of the aforesaid amounts.

In one or more embodiments, the fatty alcohol and/or fatty acid have a melting point of at least about 40° C.

In one or more embodiments, the fatty alcohol comprises or is selected from the group consisting of lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid.

In one or more embodiments, the carbon chain of the fatty alcohol or the fatty acid is substituted with a hydroxyl group.

In one or more embodiments, the fatty acid is 12-hydroxy stearic acid.

In one or more embodiments, the viscosity-modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

In one or more embodiments, the viscosity-modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

In one or more embodiments the at least one viscosity-modifying agent comprises or is selected from the group consisting of a fatty alcohol, a fatty acid and a wax, wherein the fatty alcohols and/or fatty acids have at least 12 carbon atoms in their carbon backbone. In certain embodiments the viscosity modifying agent is a combination of a fatty alcohol and a fatty acid and/or a wax.

Preferably, the fatty alcohol and/or fatty acid and/or wax are solid at ambient temperature. In certain embodiments, the fatty alcohol and/or the fatty acid and/or the wax or the mixture of them have a melting point of more than about 40° C.

### Incompatible Excipients and Undesirable Excipients

In one or more embodiments the excipients used in the gel or foam formulations are selected on the basis that when an excipient is mixed with minocycline, the minocycline does not break down significantly, as determined by a compatibility test described in the methods below.

The instability of minocycline was observed and confirmed in a compatibility study with pharmaceutical excipients described, for example, in WO11/039637. The study identified and demonstrated that different hydrophilic solvents were incompatible with minocycline, whereas certain hydrophobic solvents, emollients and waxes revealed compatibility with minocycline. Fatty alcohols, as well as some fatty acids (such as stearic acid, behenic acid, oleic acid and palmitic acid) were found to be compatible with minocycline. Additionally, some additives (aerosil and menthol) were disclosed to be compatible with minocycline, whereas surface active agents including polysorbatcs, sorbitan esters of fatty acids, polyoxyethylene alkyl ethers and polyoxyethylene esters of fatty acids were found not to be compatible. Only a few cases certain surfactants indicated some compatibility.

It was further discovered, for example, in WO11/039637 that addition of water caused rapid degradation of minocycline and addition of antioxidants (alpha-tocopherol, BHA/BHT and propyl gallate) did not prevent such degradation. Furthermore, compatible excipients became incompatible in the presence of water and addition of antioxidants did not remedy this result.

In certain embodiments, the vehicle is free of one or more of a petrolatum, surface active agents, protic solvents, certain polar aprotic solvents, isopropyl myristate, polyethylene gelling agents, polyethylene homopolymers, polyethylene copolymers, selenium derivatives and silicone thickening agents; and in certain embodiments, the foamable vehicle is substantially free of such excipients. In the context herein, the term “substantially-free” relates to a vehicle that contains a total of less than about 0.4% of a petrolatum, surface active agents, protic solvents, certain polar aprotic solvents, isopropyl myristate, polyethylene gelling agents, polyethylene homopolymers, polyethylene copolymers, selenium derivatives and silicone thickening agents cumulatively, Preferably, the vehicle comprises less than about 0.2% of two or more or all thereof by weight of petrolatum, surface active agents, protic solvents, certain polar aprotic solvents, isopropyl myristate, polyethylene gelling agents, polyethylene homopolymers, polyethylene copolymers, selenium derivatives and silicone thickening agents cumulatively or, and more preferably less than about 0.1% individually or of two or more or all thereof cumulatively. Protic solvents, such as short chain alcohols, glycols and glycerin are incompatible with tetracyclines and therefore are undesirable. Aprotic polar solvents, such as dimethyl sulfoxide (DMSO), dimethylformamide (DMF), acetonitrile, acetone, methyl ethyl ketone, 1,4-Dioxane and tetrahydrofuran (THF), N-methylpyrrolidone, pyridine, piperidine, dimethylformanide, N-methyl-2-pyrrolidone and 1-methyl-2-pyrrolidinone) and azone (1-dodecylazacycloheptan-2-one) are undesirable.

According to some embodiments, the composition is polyol free, i.e. free of polyols. A polyol is an organic substance that contains at least two hydroxyl groups in its molecular structure. In other embodiments, the composition is substantially free and comprises less than about 5% final concentration of polyols, preferably less than 2%, more preferably less than 1%; or about 1% to about 5% polyols; or about 0.5% to about 3% polyols. In some embodiments the composition comprises de minimis amounts of polyols. Where a formulation includes insignificant or de minimis amounts of polyols such as less than 0.05% it is considered to be essentially free of them.

### Surface Active Agents

For clarification, in the context herein whilst the term “standard surfactant” or “customary surfactant” refers herein to customary non-ionic, anionic, cationic, zwitterionic, amphoteric and amphiphilic surfactants. A fatty alcohol or a fatty acid and certain waxes are not regarded as a standard surfactant. However, in contrast, ethers or esters formed from such fatty alcohols or fatty acids can be regarded as a customary surfactant.

Surfactants of all kinds are undesirable in accordance with the present invention, as they are generally known to possess irritation potential.

Non-limiting examples of classes of non-ionic surfactants that are undesirable according to the present invention include: (i) polyoxyethylene sorbitan esters (polysorbates), such as polysorbate 20, polysorbate 40, polysorbate 60 and polysorbate 80; (ii) sorbitan esters, such as sorbitan monolaurate and sorbitan monooleate; (iii) polyoxyethylene fatty acid esters, such as, PEG-8 stearate, PEG-20 stearate, PEG-40 stearate, PEG-100 stearate, PEG-150 distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate; (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) PEG alkyl phenols.

As mentioned above, in the context of the present invention, while fatty alcohols, fatty acids, and certain waxes are somewhat amphiphilic, these substances are not effective as standalone surfactants that can stabilize an emulsion, let alone foamable emulsion compositions, because of their very weak emulsifying capacity and further due to their weak foaming capacity on their own.

They are occasionally used in a supporting role as co-emulsifiers, i.e., in combination with a standard surfactant but are commonly used as thickeners and have successfully been used as foam adjuvants to assist customary surfactants to boost foam quality and stability. For the purposes of forming an emulsion they are usually regarded as an oil and thus have a “required” HLB value for the purpose of determining what standard surfactant might be appropriate to use with the oil phase.

Generally, surfactants are known to possess irritation potential. One way to try and reduce or minimize potential irritation and drying of the skin or mucosa due to surfactants and their repeated use, especially when formulations are to be left on the skin or mucosa rather than being washed off, is to use essentially or primarily nonionic surfactants at significant concentrations although preferably below 5%. The current breakthrough of identifying formulations which produce gels and quality breakable foam yet omitting customary surfactants from a composition may contribute to improved tolerability of such a composition and can be an important advantage. This is especially so when a formulation is to be applied to a very sensitive target site, and particularly so on a repeated basis.

In certain embodiments, the vehicle is free of customary surfactants, or “surfactant-free” and in certain embodiments the foamable vehicle is substantially free of customary surfactants, or “substantially surfactant-free”.

In certain embodiments, the vehicle is free or substantially free of an ionic surfactant. In certain embodiments, the vehicle is free or substantially free of a zwitterionic surfactant. In certain embodiments, the vehicle is free or substantially free of a non-ionic surfactant.

### Silicone Thickening Agents

Silicone thickening agents comprise one or more polysiloxane-derived components. Such polysiloxanes are typically cross-linked and they have rubber-like characteristics, which require their solubilization in an oil, usually a silicone oil. An example of such a silicone thickening agent is ST-Elastomer 10 (Dow Corning), which is a mixture of high molecular weight dimethicone crosspolymer (12%), in cyclopentasiloxane (cyclomethicone, silicone solvent), In the context of a breakable foam, cyclomethicone is known as a defoamer and therefore its presence in high concentrations in the breakable hydrophobic vehicle is undesirable.

In one or more other specific embodiments, the vehicle is formulated substantially free of elastomers. In one or more other specific embodiments, the vehicle is formulated essentially free of elastomers. In one or more other specific embodiments, vehicle is formulated substantially free of silicones. In one or more other specific embodiments, the vehicle is formulated essentially free of silicones. In one or more other specific embodiments, the vehicle is formulated with less than about 30% silicones, or less than about 25% silicones, or less than about 20% silicones, or less than about 15% silicones, or less than about 10% silicones, or less than about 7.5% silicones, or less than about 5% silicones or less than about 2% silicones; or less than about 1% silicones; or less than about 0.5% silicones; or about 1% to about 5% silicones or about 0.5% to about 3% silicones. In one or more other specific embodiments, the vehicle does not comprise a silicone other than cyclomethicone. In one or more other embodiments the drug carrier does not comprise one or more volatile silicones. In other embodiments volatile silicones are present at about 3% or less.

In one or more embodiments, semi-solid hydrophobic oils are a subsidiary component in the vehicle, for example being present at less than about 45%, at less than about 40%, at less than about 35%, at less than about 30%, at less than about 25%, less than about 20%, less than about 15%, less than about 10%, or less than about 5% by weight of the vehicle. In one or more alternative embodiments, semi-solid oils are omitted.

In one or more embodiments, the hydrophobic vehicle is substantially free of at least one or more selected from a group consisting of surface active agents, pharmaceutically active agents, and silicone thickening agents.

In one or more embodiments, the hydrophobic vehicle is substantially free of at least one or more selected from a group consisting of surface active agents, polymeric gelling agents, pharmaceutically active agents, and silicone thickening agents.

In one or more embodiments, the hydrophobic vehicle is essentially free of at least one or more selected from a group consisting of surface active agents, polymeric gelling agents, pharmaceutically active agents, and silicone thickening agents.

In one or more embodiments, the hydrophobic vehicle contains less than about 0.4% by weight of the vehicle; or less than about 0.2% by weight of the vehicle; or less than about 0.1% by weight of the vehicle of one of or a combination of two, three or all of surface active agents, polymers, pharmaceutically active agents, and silicone thickening agents

### The Composition Essential Ingredients

In certain embodiments, a hydrophobic solvent can be useful. For example, some essential oils can kill microorganisms or may prevent of conditions that involve microbial infection. Additionally, hydrophobic solvents can useful for the treatment of conditions which involve damaged skin, such as psoriasis or atopic dermatitis. The use of hydrophobic based water-free formulation can maximize the antimicrobial potentials of the formulations.

Fatty alcohols can also be of possible help. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and anti-inflammatory properties (see, U.S. Pat. No. 4,874,794). Longer chain fatty alcohols, e.g., tetracosanol, hexacosanol, heptacosanol, octacosanol, triacontanol, etc., are also known for their metabolism modifying properties, and tissue energizing properties.

The combination of a hydrophobic solvent and a fatty alcohol or fatty acid may be of possible help in conditions characterized, for example, by infection and/or inflammation.

The foamable vehicle is suitable for use in the manufacture of a medicament including a placebo or color agent or a cosmetic agent.

The topical vehicles of the present invention avoid, reduce, minimize or or do not cause adverse events, side effects or skin irritation, which are attributed to oral tetracycline antibiotics. Photosensitivity, for example, is a known side effect of oral minocycline. It is manifested as an exaggerated sunburn reaction on areas of the body exposed to direct sunlight or ultraviolet light, resulting in muddy brown skin discoloration. Use of oral minocycline over an extended period of time can also lead to skin pigmentaton e.g. manifested as blue-gray skin and blue-gray staining in areas of scaring and inflammation associated with acne. Drug related pigmentation was not observed during the period of topical application of the vehicle or on follow up. Tooth staining potential of oral minocycline in adult populations has also been acknowledged in recent literature. In contrast, no tooth staining was reported during the period of topical application of the vehicle foam or on follow up. So in one or more embodiments the topical vehicle foam avoids tooth staining.

In one or more embodiments the method is useful for treating acne, including administering topically to a surface having the disorder a hydrophobic vehicle as described above, wherein:

(a) the at least one hydrophobic solvent comprises or is selected from a group consisting of a soybean oil, a coconut oil, a cyclomethicone, a light mineral oil, and mixtures thereof;

(b) the at least one viscosity modifying agent comprises or is selected from a group consisting of a fatty acid, a fatty alcohol, a wax, a hydrogenated castor oil, and mixtures thereof.

In one or more embodiments the disorder is acne or acne related or has acne like symptoms.

In one or more embodiments, the vehicle further comprises fumed or modified silica (SiO₂) such as Aerosil R972.

In one or more embodiments of the invention, there is disclosed a method for treating a disorder of the pilosebaceous unit, including acne, including administering topically to a surface having the disorder a hydrophobic vehicle substantially free of surfactants, and/or substantially free of surfactants and polymeric agents as described above, wherein

(a) the at least one hydrophobic solvent comprises or is selected from a group consisting of a soybean oil, a coconut oil, a cyclomethicone, a light mineral oil, and mixtures thereof;

(b) the fatty alcohol comprises or is selected from a group consisting of cetostearyl alcohol, myristyl alcohol, stearyl alcohol, behenyl alcohol, and mixtures thereof;

the fatty acid comprises or is selected from the group consisting of stearic acid, beeswax, a hydrogenated castor oil, and mixtures thereof;
the wax comprises or is selected from the group consisting of bees wax, a hydrogenated castor oil, and mixtures thereof.

In one or more embodiments the above hydrophobic vehicle is used to treat one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections.

Also provided herein is a method for treating human skin diseases or disorders especially for the treatment of acne, rosacea and/or superficial infections, including skin infections, such as impetigo, including administering topically to a surface having the disease or disorder a hydrophobic vehicle containing:

(a) a mixture of soybean oil in an amount of about 50 weight percent, coconut oil in an amount of about 24 weight percent, cyclomethicone in an amount of about 5 weight percent, and light mineral oil in an amount of about 4 weight percent;

(b) a mixture of about 3.5 weight percent cetostearyl alcohol, about 2.5 weight percent myristyl alcohol, about 1.5 weight percent stearyl alcohol, about 1 weight percent behenyl alcohol, about 3 weight percent stearic acid, about 2 weight percent beeswax, and about 2 weight percent hydrogenated castor oil;

(c) fumed (modified) silica in an amount of about 0.25 weight percent; and

(d) color agent or cosmetic agent an amount of about 1.0 weight percent.

In one or more embodiments of the invention is disclosed a method for treating human skin disorders or diseases. In one or more embodiments a method of treating one or more of acne, acne related symptoms, a tetracycline antibiotic responsive acne related disorder, a tetracycline antibiotic responsive skin disorder, skin disorder caused by a bacteria, a tetracycline antibiotic responsive disorder, a sebaceous gland disorder, P. acne bacteria associated disorders and superficial infections, including skin infections, including administering topically to a surface having the disorder a hydrophobic vehicle substantially free of surfactants, and/or substantially free of surfactants and polymeric agents as described above, containing:

(a) a mixture of soybean oil in an amount of about 50 weight percent, coconut oil in an amount of about 23.6 weight percent, cyclomethicone in an amount of about 5 weight percent, and light mineral oil in an amount of about 4 weight percent;

(b) a mixture of about 3.5 weight percent cetostearyl alcohol, about 2.5 weight percent myristyl alcohol, about 1.5 weight percent stearyl alcohol, about 1 weight percent behenyl alcohol, about 3 weight percent stearic acid, about 2 weight percent beeswax, and about 2 weight percent hydrogenated castor oil;

(c) modified (fumed) silica (Aerosil R 972) in an amount of about 0.25 weight percent; and

(d) a color agent or a cosmetic agent in an amount of about 1.44 weight percent.

In one or more embodiments, any vehicle of the present invention can also contain a fragrance. In one or more embodiments, the fragrance is at a concentration of about 0.1% by weight to about 1% by weight.

In one or more embodiments, the vehicle comprises about 48% w/w to about 51% w/w of soybean oil. In one or more embodiments, the composition comprises about 23% w/w to about 24% w/w of coconut oil. In one or more embodiments, the composition comprises about 4% w/w to about 6% w/w of cyclomethicone. In one or more embodiments, the composition comprises about 1% w/w to about 5% w/w of light mineral oil.

In one or more embodiments, the vehicle comprises about 3% w/w to about 4% w/w of cetostearyl alcohol. In one or more embodiments, the composition comprises about 2% w/w to about 4% w/w of stearic acid. In one or more embodiments, the vehicle comprises about 2% w/w to about 3% w/w of myristyl alcohol. In one or more embodiments, the vehicle comprises about 1% w/w to about 2% w/w of stearyl alcohol. In one or more embodiments, the vehicle comprises about 0.5% w/w to about 1.5% w/w of behenyl alcohol. In one or more embodiments, the vehicle comprises about 1% w/w to about 3% w/w of hydrogenated castor oil. In one or more embodiments, the vehicle comprises about 1% w/w to about 3% w/w of beeswax.

In one or more embodiments, the vehicle comprises about 0.1% w/w to about 0.3% w/w of fumed (modified) silica. In one or more embodiments, the vehicle comprises about 0.05% w/w to about 4% w/w of color agent or a cosmetic agent. In one or more embodiments, the vehicle comprises about 3% w/w to about 15% w/w of propellant based on the weight of the total composition.

In one or more embodiments, there is provided a method for treating acne vulgaris, including administering topically to a surface having acne vulgaris a vehicle which is highly effective against bacteria. In one or more embodiments the vehicle is effective against some multi-drug resistant strains (e.g., antibiotic-resistant P. acnes).

In one or more embodiments, there is provided a method for treating acne vulgaris, including administering topically to a surface having acne vulgaris a vehicle which is highly effective against antibiotic-resistant P. acnes bacteria.

In one or more embodiments, there is provided a method for treating acne vulgaris, including administering a vehicle topically, once a day, to a surface having acne vulgaris.

In one or more embodiments, there is provided a method for treating acne vulgaris, including administering a vehicle topically, twice a day, to a surface having the acne vulgaris.

In one or more embodiments, there is provided a method for treating acne vulgaris, including administering a vehicle topically, alternate-day or intermittently, to a surface having acne vulgaris.

In one or more embodiments, there is provided a method for treating acne vulgaris, including administering a vehicle topically, gradual reduction to a lower maintenance dose, which can be increased if further outbreaks occur, to a surface having acne vulgaris. In one or more embodiments, a maintenance dose can be applied topically, daily, alternate daily, twice weekly or weekly for a month, two months, quarterly, six months or indefinitely. In one or more embodiments the maintenance dose may be commenced after four weeks of treatment, or after five weeks of treatment, or after six weeks of treatment, or after seven weeks of treatment, or after eight weeks of treatment, or after nine weeks of treatment, or after ten weeks of treatment, or after eleven weeks of treatment, or after twelve weeks of treatment, or after thirteen weeks of treatment, or after fourteen weeks of treatment, or after fifteen weeks of treatment, or after sixteen weeks of treatment.

In one or more embodiments, there is provided a method for treating acne vulgaris, including administering a vehicle topically, once daily for at least six weeks, to a surface having acne vulgaris.

In one or more embodiments, there is provided a method for treating acne vulgaris, including administering a vehicle topically, once daily up to six weeks, to a surface having the acne vulgaris.

In one or more embodiments, there is provided a method for treating acne vulgaris, including administering a vehicle topically, once daily for twelve weeks or less than twelve weeks, to a surface having acne vulgaris.

In one or more embodiments, there is provided a method for treating acne vulgaris, including administering a vehicle topically, once daily for six weeks or less than six weeks, to a surface having acne vulgaris.

In one or more embodiments, there is provided a method for treating acne vulgaris, including administering topically a vehicle, once daily for three weeks or less than three weeks, to a surface having acne vulgaris.

One known disadvantage of state of the art compositions (for example Retin A) is that they must be administered for at least seven weeks before consistent beneficial effects are observed, thus making it burdensome for use. It is therefore an advantage of the vehicle provided herein is that they can be effective when administered once daily for only six weeks. In certain embodiments, the vehicle may further be effective even if administered alternate-day according to the condition of the patient. In other embodiments, the vehicle may be used even if administered more than once a day and/or for more than twelve weeks according to the condition of the patient.

Another disadvantage of state of the art compositions (such as Akne Mycine) is that they have an ointment base, comprising petrolatum which is greasy and generally considered less usable in the case of facial treatment of acne. Another disadvantage of state of the art compositions is that they contain surfactants, which can be irritants. In some cases, irritation at the application site has been reported with the use of such compositions.

It is therefore an advantage of the vehicle provided herein that they are breakable gels or foams; and therefore are easy to apply to the skin and also avoid skin irritation that has been associated with compositions containing surfactants.

Topical compositions must stay on the skin for a sufficient period of time so they can be absorbed onto the skin, to perform their activity and to further exert a preventative effect. They should preferably not irritate the skin; and they should be perceived by the patient as pharmaceutically convenient in order to achieve sufficient patient compliance. By “pharmaceutically convenient”, it is meant that the skin look and feel to the patient is good, i.e., it must not be too watery or too greasy and it must easily be applied.

Foam is extremely advantageous in the topical treatment of skin diseases, especially in teenagers with skin afflicted with acne, since it is light and easy to apply and collapses and spreads with a minor mechanical force like a simple rub. When dispensed, even in small quantities, drug delivery in the form of foam can also cover a larger surface area of application while also facilitating better product application in areas where conventional topical products cannot be as effective. Foam absorbs rapidly—without the need of repeated rubbing—which is helpful and important for treatment of damaged or irritated skin, sores, and lesions.

Thermally stable foam which breaks upon application of mild shear force is extremely advantageous in the topical treatment of skin diseases. It can be applied directly onto skin or hands of the patient without collapsing. So hydrophobic vehicle according to the description provided herein, facilitates easy application and even distribution thereof, thereby improving treatment convenience. In contrast, Evoclin foam is a temperature sensitive foam that collapses immediately on the skin so it must first be applied onto a cool surface and then quickly applied using fingertips onto the surface which impedes patient compliance.

The formulation packaged into an aerosol container is devoid of any contact with air, light, or any other form of contamination as it is a completely sealed system throughout the life of the product. Thus, light and oxidation sensitive actives can be stabilized effectively in the aerosol system.

In one or more embodiments there is provided a method of administering vehicle to a target area such as skin of a patient comprising releasing foam, applying it to the area, and collapsing the foam. In one or embodiments, the foam is applied by spreading. In the course of spreading mechanical shear can cause the foam to collapse. In one or more embodiments, the collapsed foam is not washed off. In one or more embodiments it is absorbed onto the area of skin. In one or more embodiments it avoids skin irritation or an ointment sensation.

Breakable gels, which comprise liquid oils and a thickening agent, are also very convenient for use, as they liquefy on application of mild shear force such as gentle rubbing, and in tam, they readily absorb onto the skin.

In one or more embodiments, there is provided a method of applying a gel vehicle to an area of skin of a patient comprising releasing a gel, applying it to the area, and collapsing or liquefying the gel. In one or more embodiments, the collapsed or liquefied gel is not washed off. In one or more embodiments, the collapsed or liquefied gel is readily absorbed and does not leave an ointment sensation.

In one or more embodiments, a gel or a liquid gel or a collapsed foam is absorbed within 240 seconds, or within 200 seconds, or within 180 seconds, or within 150 seconds, within 120 seconds, or within 100 seconds, or within 80 seconds, or within 60 seconds, or within 50 seconds, or within 40 seconds, or within 30 seconds, or within 20 seconds, or within 10 seconds, or within 5 seconds, or within 2 seconds or less. By absorbed is meant that the vehicle enters onto and into an area of skin, mucosa or eye, often forming a thin coating on the surface.

Thus, it is possible to use a composition devoid of active agents thereby reducing toxicity and increasing safety and skin tolerability.

In an embodiment skin disorders and diseases can be treated with a vehicle according to the present invention such as rosacea, wounds, burns, inflammatory skin dermatoses superficial infections, including skin infections, such as impetigo, antibiotic responsive dermatoses and sebaceous gland disorders.

In one or more embodiments, there is provided a method for treating acne, including administering topically, to a surface having acne, a vehicle comprising a composition essentially free of pharmaceutically active agents, wherein a reduction in the number of lesions is observed after six weeks or less than six weeks of treatment compared to baseline. In one or more embodiments, there is provided a method for treating acne, including administering topically, to a surface having acne, with a vehicle, wherein an improvement in the skin condition is observed after six weeks or less than six weeks of treatment and wherein an improvement is considered as restoration of visible, normal cutaneous topographic features, indicating the return of skin integrity. In an embodiment the improvement is after three weeks, or after four weeks, or after five weeks, or after six weeks or after seven weeks, or after eight week, or after 9 weeks, or after, ten weeks, or after 11 weeks, or after 12 weeks.

In one or more embodiments, there is provided a method for treating P. acnes and reducing inflammation, thereby reducing the number of inflammatory acne lesions by applying topically an effective amount of vehicle which is in the form of a gel, liquid gel or foam to an afflicted area of a patient in need. In one or more embodiments, there is provided a method for eradicating P. acnes and reducing inflammation, thereby reducing the number of inflammatory acne lesions. In one or more embodiments, there is provided a method for reducing the number of non-inflammatory acne lesions, by applying topically an effective amount of with a vehicle which is in the form of a gel, liquid gel or foam to an afflicted area of a patient in need. In one or more embodiments, the method involves applying a gel, liquid, gel or foam formulation topically to a target surface in need of treatment and breaking the gel or foam over the target site. In one or more embodiments, the method uses a once daily dosage regime for twelve weeks or less than twelve weeks. In one or more embodiments the twelve week dosage regime is followed by a once daily maintenance dose for one, two, three or more weeks according to the condition and response of the patient. In one or more embodiments, the method uses a once daily dosage regime for six weeks or less than six weeks. In one or more embodiments the six week dosage regime is followed by a once daily maintenance dose for one, two, three or more weeks according to the condition and response of the patient. In one or more embodiments, the method uses a once daily dosage regime of for six weeks or less than six weeks followed by a once weekly maintenance dose for one, two, three, four, five, six, seven, eight, nine, ten, eleven and or more weeks according to the condition and response of the patient. In one or more embodiments, the method uses a once daily dosage regime of for three weeks or less than three weeks followed by a once weekly maintenance dose for one, two, three, four, five, six, seven, eight, nine, ten, eleven or more weeks according to the condition and response of the patient. In one or more embodiments, the method uses a once daily dosage regime of for two weeks followed by a daily maintenance dose for one, two, three or more weeks according to the condition and response of the patient. In one or more embodiments the method uses a once daily dosage regime of for twelve weeks wherein the treatment is every alternate week.

In one or more embodiments, the method uses an additional step of pre cleaning and drying the lesions and surrounding area before applying the gel, liquid gel or foam vehicle.

In one or more embodiments, the method uses a sterile applicator or prior to the steps of administering and/or collapsing and/or spreading, the hands of the person spreading are sterilized in order to avoid cross contamination where there is a systemic as well as a topical bacterial infection.

The vehicle was manufactured under current Good Manufacturing Principles (cGMP) conditions and provided in aluminum aerosol canisters mounted with valve and actuator. Each canister was filled with 25 g of product and 3 g of propellant. Upon actuation of the canister an aliquot of quality foam was released.

A multi-center randomized double blind placebo controlled parallel group, dose finding Phase II clinical study, conducted in patients afflicted with acne is reported in Example 3 below. The study was designed to assess the efficacy, safety and tolerability of foamable composition comprising minocycline at one of two different concentrations (strengths): a lower concentration of minocycline of 1% by weight of the formulation and higher concentration of minocycline 4% by weight of the formulation, in comparison with placebo (i.e. vehicle). The placebo-control design was based on the FDA guidance designated eligibility criteria and endpoints “Guidance for Industry—Acne Vulgaris: Developing Drugs for Treatment (2005).” The concentrations of minocycline in the composition were selected according to formulation integrity and stability considerations.

The study included six scheduled study visits in which the patients were evaluated: Day 1 (Visit 1) Baseline which included, screening and treatment initiation, Week 3 (Visit 2), Week 6 (Visit 3), Week 9 (Visit 4), Week 12 (Visit 5) End of Treatment (EOT) and Week 16 (Visit 6) “follow up” (F/U). Safety, tolerability and efficacy evaluations were done at baseline, interim visits (2 to 4), EOT and F/U.

The 150 patients starting the clinical trial had on average moderate severe to severe acne with an mean number of inflammatory lesions of 33-36 and an mean number of non-inflammatory lesions of 42-46. So patients started the study with about 75-82 lesions. The effects observed were dose dependent. The results seen with 4% minocycline were better than those with 1% minocycline which in turn were better than the vehicle composition without minocycline (the “vehicle” or “placebo formulation” or “placebo”) which was used as the placebo. Nevertheless, the vehicle had a substantial and unexpected positive effect. For example, as described below, following daily application a substantial reduction of acne lesions was observed. The effect of the vehicle without minocycline may be a contributing factor in the success observed with the 1% and 4% Minocycline formulations as it may act as a springboard or platform from which the antibiotic can have its therapeutic effect.

By way of a non-binding analogy someone standing on a chair has less distance to go to reach the ceiling. By having an effective vehicle topical minocycline can achieve more for the skin than oral minocycline.

Without being bound by any theory, it is thought that the selection of excipients that are compatible with minocycline may have contributed to the effect of the placebo formulation. One of more of the excipients in the vehicle may have a therapeutic effect topically on their own and may also act together with other excipients and or an active pharmaceutical ingredient, such as, a tetracycline antibiotic to amplify the effect and thereby achieving two incremental advantages over oral therapy. One being an improved clinical response and the other being avoiding systemic side effects. Other possible theories for the placebo effect include the application of oils to the skin. Although this may run counter to current thinking that oily material should be avoided, the presence of such materials may actually help improve the skin, and/or extract sebaceous matter from the gland or pores and/or have a negative feedback so as to reduce the production of material that can block or interfere with the operation of the sebaceous glands. Other contributing factors may include the presence of fatty alcohols; and/or the presence of a fatty acid; and/or the presence of waxes in the formulation.

Clinical trial results of acne patients treated with the vehicle have now surprisingly demonstrated that once daily topical administration of the vehicle lead to rapid reduction in the number of non-inflammatory and inflammatory acne lesions. A reduction in both inflammatory and non-inflammatory lesions could be observed from about 3 weeks with improvement continuing to 12 weeks. Non inflammatory lesions responded, in general, slightly better that than non-inflammatory lesions. The effect of treatment on reducing the number of lesions and improving the patient's skin appeared to approach a steady state between 6-12 weeks for inflammatory lesions and 6-12 for non-inflammatory. Treatment was stopped at twelve weeks but the patients were seen again 4 weeks after cessation of treatment at week 16. Surprisingly, the effect of the previous 12 weeks of treatment on reducing the number of lesions and improving the patient's skin was observed to continue in the absence of treatment with minor decrease in the mean number of lesions. In other words four weeks after cessation the patient's skin did not appear to show signs of relapse.

Reduction of 49% and 50% in the number of non-inflammatory and inflammatory acne lesions respectively within only six weeks of treatment with the vehicle was demonstrated. A further reduction of about 57% in the number of non-inflammatory lesions and 51% in inflammatory acne lesions was observed after twelve weeks of treatment.

Unexpectedly, in the course of treatment the vehicle was able to reduce symptoms and severity of acne. Improvement was apparent as was the restoration of visible, normal cutaneous topographic features, indicating the return of skin integrity.

The percentage reduction in inflammatory lesions and also in non-inflammatory lesions was found to be comparable and even better to than seen with other current acne treatments. For example application of vehicle resulted in a 51% mean reduction of inflammatory lesions and 57% of non-inflammatory lesions respectfully at 12 weeks. In contrast, oral minocycline (Solodyn™) reported a reduction of 44% in inflammatory lesions and no effect on non-inflammatory lesions. The reduction in the percentage of inflammatory lesions demonstrated with the vehicle is greater than the reduction recorded for oral minocycline. The reduction in non-inflammatory lesions demonstrated with the vehicle is greater than for four recently approved topical products which use active ingredients other than tetracycline antibiotics, namely Epiduo™, Acanya™ Fabior™ and Ziana™. So apart from the avoidance of unwanted systemic effects, adverse events and skin irritation, topical vehicle treatment appears to have substantial advantages over oral minocycline treatment of acne and other available topical treatments.

The Investigators Global Assessment (“IGA”) of the vehicle was also very encouraging. The percentage of patients, for example, with an IGA at 12 weeks of “almost clear” or “clear” was 20% for the placebo. Even after treatment ceased patients IGA score continued to improve to 33%. Approximately a third of subjects who received vehicle foam had ‘excellent’ improvement and approximately 60% of subjects had ‘excellent’ or ‘moderate’ improvement as assessed by the physician after twelve weeks of treatment. Approximately 28% of subjects who received the vehicle foam evaluated their acne as “much better than prior to study”, and over 75% who received the vehicle foam evaluated their acne as “slightly better than prior to study”.

The positive and unexpected results described herein in relation to the vehicle and acne are the more remarkable as a prevailing view in relation acne treatment is to avoid applying oily products to the skin since these results were obtained with an oil based vehicle.

The patient feedback was likewise positive. High overall patient satisfaction was also reported. No drug related systemic side effects were observed. The product is considered safe and there were only two mild skin related adverse events reported. See Table 8-6-1 providing data relating irritation events i.e. the lack thereof.

All skin irritation events were mostly mild and transient and did not require discontinuance treatment. For example, one case of mild erythema and one case of moderate erythema were reported in visit 2 and both disappeared already at visit 4. One case of mild pigmentation was reported in visit 3 and disappeared already at visit 4. However, the Investigator described these pigmentation cases as localized post inflammatory pigmentation, typical to the natural healing process of acne lesions. No itching was reported.

A month after the end of treatment one patient manifested peeling and another manifested skin dryness. It is postulated that these post treatment skin irritation symptoms cannot be directly linked to the placebo but rather may be a result of not applying oil provided by the formulation.

So, the vehicle appears to avoid or minimize known side effects and skin irritation and may act to prevent or minimize skin irritation, thereby leading to better patient compliance compared to available treatment options. Thus, vehicle offers a safe, user friendly and effective alternative to current oral minocycline treatments and other topical treatments. Moreover it can provide a shorter treatment regime (e.g. six weeks) with comparable or even better efficacy results to available treatment options with regard to inflammatory lesion and enhanced efficacy results with regard to non-inflammatory lesion while avoiding unwanted side effects, drug related adverse events and skin irritation. In one or more embodiments good tolerability is demonstrated with relatively few reports of skin irritation including, erythema, dryness or peeling. In one or more embodiments good tolerability is demonstrated with relatively few or no reports of pigmentation or itching.

It was also surprising to note that clinical studies affirmed that the once-daily treatment regimen with vehicle foam was safe and well tolerated even for a prolonged treatment period. During twelve weeks of treatment no serious adverse events were reported, no occurrences of pigmentation or itching were noted and only a few occurrences of erythema, skin dryness and peeling were recorded in acne vulgaris patients. This is advantageous as most approved topical prescription treatments currently available to treat acne vulgaris are in the form of creams and ointments and are associated with skin irritation symptoms such as pigmentation, erythema, dryness, peeling and itching which impede patient compliance. The present vehicle meet the long felt need for a convenient safe method of treatment of acne which avoids skin irritation and minimizes or avoids adverse events or side effects associated with other treatments including oral tetracyclines even when applied for a prolonged period while maintaining efficacy.

Most approved topical prescription treatments currently available to treat acne vulgaris require a twelve week treatment regimen, which may impact patient compliance. In contrast, the present gel, liquid gel and foamable vehicle compositions meet a long felt need for a shorter treatment regimen having an earlier onset, and a higher percentage reduction in lesions while maintaining high levels of safety, tolerability and efficacy.

As with other therapeutic regimens, patient compliance is essential in the effectiveness of prescribed antibiotics. With poor compliance, therapeutic goals are less likely to be achieved, resulting in poorer patient outcomes. Poor compliance is associated with deteriorating skin condition, the need for additional consultations, the emergence of bacterial resistance, extra drugs, additional expenses on cosmeticians and increases in direct and indirect costs of healthcare management.

In general, patients are more compliant with simple and shorter dosing regimens. Both the dosage schedule and the patient's daily routine should be considered when prescribing antibiotics. The vehicle may also be more attractive than oral therapy because they reduce the potential for systemic side effects, typically nausea and diarrhea, which are commonly associated with many systemic antibiotics.

In one or more embodiments there is provided a method for treating acne, including administering topically, to a surface having acne, a vehicle administered at least alternate days or once daily which has a high or improved patient compliance compared with existing treatments.

In one or more embodiments one or more of the methods herein of treating or alleviating acne or acne vulgaris can also be used for treating a disorder including one or more of the following: acne related or associated disorder, acne like symptoms, acne related symptoms, a tetracycline antibiotic responsive acne related disorder, skin disorder caused by a bacteria, and a tetracycline antibiotic responsive sebaceous gland disease, P. acne bacteria associated disorders and other superficial infections, including skin infections.

In one or more embodiments there is provided a method of maintenance therapy, to prevent acne recurrence or reduce the severity of the acne recurrence, applied to a patient in need which comprises applying to the skin on a regular basis (as defined above) a hydrophobic gel or foam vehicle.

In one or more embodiments there is provided a regime or regimen for treating a patient having one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections which comprises applying to the afflicted area on a regular basis a hydrophobic gel or foam composition or vehicle.

In one or more embodiments there is provided the use of a vehicle for the manufacture of a medicament for treating one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections in a human subject in need thereof, wherein the vehicle is to be administered topically to said human subject in an amount that is effective to treat acne.

In one or more embodiments there is provided a vehicle for use as a medicament in treating and/or preventing one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, P and/or acne bacteria associated disorders, and other superficial infections, including skin infections wherein the vehicle administered topically at least alternate days or at least once daily for at least six weeks.

In one or more embodiments there is provided a hydrophobic gel or foam vehicle agents for use in treating acne in a human subject suffering therefrom comprising topically administering the composition to the human subject in a sufficient amount and for a sufficient time to decrease the number of acne lesions by at least 25%.

In one or more embodiments there is provided a hydrophobic gel or foam vehicle for use in treating one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections, wherein vehicle is administered topically at least alternate days or at least once daily for twelve weeks or less than twelve weeks of treatment.

In one or more embodiments there is provided a hydrophobic gel or foam vehicle for use in treating acne in a human subject comprising topically administering the composition at least alternate days or at least once daily, wherein a decrease the number of acne lesions is observed after at least six weeks of treatment.

In one or more embodiments there is provided a hydrophobic gel or foam vehicle for use in treating a disorder selected from the group consisting of acne, and/or acne related symptoms, and/or a tetracycline antibiotic responsive acne related disorder, a tetracycline antibiotic responsive skin disorder, and/or skin disorder caused by a bacteria, a and/or tetracycline antibiotic responsive disorder, and/or a sebaceous gland disorder, and/or P. acne bacteria associated disorders and other superficial infections, including skin infections, wherein the vehicle is administered topically at least alternate days or at least once daily for at least six weeks to the skin, wherein the hydrophobic gel or foam vehicle, is waterless, is surfactant free and does not comprise a silicone other than cyclomethiconc.

In one or more embodiments there is provided a hydrophobic foam or gel vehicle for use in retarding, arresting, or reversing the progression of one or more of acne, and/or acne related symptoms, and/or a tetracycline antibiotic responsive acne related disorder, and/or a tetracycline antibiotic responsive skin disorder, and/or skin disorder caused by a bacteria, and/or a tetracycline antibiotic responsive disorder, and/or a sebaceous gland disorder, and/or P. acne bacteria associated disorders and other superficial infections, including skin infections, wherein the hydrophobic foam vehicle or gel is applied topically to the skin at least alternate days or at least once a day for at least six weeks.

In one or more embodiments the human subject is 30 or less than 30 years old, or is 25 or less than 25 years old, or is 22 or is less than 22 years old, or is 20 or less than 20 years old, or is 18 or less than 18 years old, or 15 or is less than 15 years old, or is between 8 to 25 years old or is between 9 to 22 years old. In an embodiment the subject is a female. In an embodiment the female is under the age of forty six and optionally is a pregnant or breastfeeding female. In an embodiment the subject is a male. In an embodiment the subject is a teenager. In another embodiment the subject is a child.

In certain embodiments the treatment is applied on average once daily for two weeks, or for three weeks or for four weeks or for five weeks or for six weeks or for seven weeks or for eight weeks or for nine weeks or for ten weeks or for eleven weeks of for some other period of less than twelve weeks.

In one or more embodiments there is provided a method for treating acne (the disorder), including administering topically, to a surface having the disorder, a vehicle, wherein essentially no skin irritation or no serious adverse events or no drug related adverse events are observed. In one or more embodiments good tolerability is demonstrated with relatively few reports of skin irritation including, erythema, dryness or peeling. In one or more embodiments good tolerability is demonstrated with relatively few or no reports of pigmentation or itching.

In one or more embodiments there is provided a method for treating acne, including administering topically, to a surface having acne, a vehicle, wherein an enhanced efficacy of the topical compositions is demonstrated.

In one or more embodiments, there is provided a method for treating acne vulgaris, including administering topically to a surface having acne a vehicle, wherein after twelve weeks of treatment, at least about 40% of the treated acne vulgaris lesions disappear (in other words, a 40% decrease in the number of lesions) so that no further antimicrobial therapy is necessary. In some embodiments, at least about 50%, at least about 60%, at least about 70% or at least about 80% of the treated acne vulgaris lesions disappear. In one or more embodiments, at least about 90% of the treated acne vulgaris lesions disappear.

In one or more embodiments, there is provided a method for treating acne vulgaris, including administering topically to a surface having acne a vehicle, wherein after six weeks or less than six weeks of treatment, at least about 45% of the treated acne vulgaris lesions disappear (in other words, a 45% decrease in the number of lesions) so that no further antimicrobial therapy is necessary. In some embodiments, at least about 50%, at least about 60%, at least about 70% or at least about 80% of the treated acne vulgaris lesions disappear after six week or less than six weeks of treatment. In one or more embodiments, at least about 90% of the treated acne vulgaris lesions disappear after six week or less than six weeks of treatment.

In one or more embodiments there is provided a method for treating acne, including administering topically, to a surface having acne, a vehicle, wherein a percent of total number lesions that disappeared is at least about 30%, at least about 40%, or at least about 50% after three weeks or less than three weeks of treatment.

In one or more embodiments there is provided a method for treating acne, including administering topically, to a surface having acne, a vehicle, wherein a percent of inflammatory lesions that disappeared is at least about 30%, or at least about 35%, or at least about 40%, or at least about 45%, or at least about 50% after three weeks or less than three weeks of treatment.

In one or more embodiments there is provided a method for treating acne, including administering topically, to a surface having acne, a vehicle, wherein a percent of non-inflammatory lesions that disappeared is at least about 30%, or at least about 35%, or at least about 40%, or at least about 45%, or at least about 50% after three weeks or less than three weeks of treatment.

In one or more embodiments there is provided a method for treating acne, including administering topically, to a surface having acne, a vehicle, wherein a percent of total number lesions that disappeared is at least about 30%, or at least about 35%, or at least about 40%, or at least about 45%, or at least about 50% after four weeks or less than four weeks of treatment.

In one or more embodiments there is provided a method for treating acne, including administering topically, to a surface having acne, a vehicle, wherein a percent of total number lesions that disappeared is at least about 30%, or at least about 35%, or at least about 40%, or at least about 45%, or at least about 50% after five weeks or less than five weeks of treatment.

In one or more embodiments there is provided a method for treating acne, including administering topically, to a surface having acne, vehicle, wherein a percent of total number lesions that disappeared is at least about 30%, or at least about 35%, or at least about 40%, or at least about 45%, or at least about 50% after six weeks or less than six weeks of treatment.

In one or more embodiments there is provided a method for treating acne, including administering topically, to a surface having acne, a vehicle, wherein a percent of total number lesions that disappeared is at least about 30%, or at least about 35%, or at least about 40%, or at least about 45%, or at least about 50% after seven weeks or less than seven weeks of treatment.

In one or more embodiments there is provided a method for treating acne, including administering topically, to a surface having acne, a vehicle, wherein a percent of total number lesions that disappeared is at least about 30%, or at least about 35%, or at least about 40%, or at least about 45%, or at least about 50% after eight weeks or less than eight weeks of treatment.

In one or more embodiments there is provided a method for treating acne, including administering topically, to a surface having acne, a composition which essentially free of pharmaceutically active agents, wherein a percent of total number lesions that disappeared is at least about 30%, or at least about 35%, or at least about 40%, or at least about 45%, or at least about 50% after nine weeks or less than nine weeks of treatment.

In one or more embodiments, there is provided a method for treating acne, including administering topically, to a surface having acne, a vehicle wherein a percent of total number lesions that disappeared is at least about 45%, or at least about 50%, or at least about 60%, or at least about 70%, or at least about 80% after four weeks after the end of the treatment.

In one or more embodiments there is provided a method for treating acne, including administering topically, to a surface having acne, vehicle, wherein a percent of total number lesions that disappeared is at least about 30%, or at least about 35%, or at least about 40%, or at least about 45%, or at least about 50%, or at least about 55%, or at least about 60%, or at least about 70%, or at least about 80% after six weeks or after less than six weeks of treatment.

In one or more embodiments there is provided a method for treating acne, including administering topically, to a surface having acne, a vehicle, wherein a percent of total number lesions that disappeared is at least about 30%, or at least about 35%, or at least about 40%, or at least about 45%, or at least about 50%, or at least about 55%, or at least about 60%, or at least about 70%, or at least about 80% after nine weeks or after less than nine weeks of treatment.

In one or more embodiments there is provided a method for treating acne, including administering topically, to a surface having acne a vehicle, wherein a percent of total number lesions that disappeared is at least about 30%, or at least about 35%, or at least about 40%, or at least about 45%, or at least about 50%, or at least about 55%, at least about 60%, or at least about 70%, or at least about 80% after twelve weeks or after less than twelve weeks of treatment.

In one or more embodiments there is provided a method for treating acne, including administering topically, to a surface having acne, a vehicle, wherein a percent of total number lesions that disappeared at the end of treatment is statistically significant compared to baseline.

In one or more embodiments there is provided a method for treating acne, including administering topically, to a surface having acne, a vehicle wherein a percent of total number lesions that disappeared at the end of treatment compared to baseline is statistically significant when compared to 1% or 4%.

In one or more embodiments there is provided a method for treating acne, including administering topically, to a surface having acne a vehicle, wherein a percent of total number of inflammatory lesions and non-inflammatory lesions that disappeared at the end of treatment in placebo compared to baseline is higher than in both 1% or 4% dose groups.

In one or more embodiments there is provided a method for treating acne, including administering topically, to a surface having acne a vehicle, wherein a percent of total number of inflammatory lesions and non-inflammatory lesions that disappeared at the end of treatment in both 1% or 4% dose groups compared to baseline is higher than in placebo groups.

In one or more embodiments the vehicle or placebo formulation has a beneficial effect. In one or more embodiments there is provided a method for treating acne, including administering topically, to a surface having acne, a placebo composition being a vehicle composition described herein for the delivery of a tetracycline that does not comprise a tetracycline antibiotic, wherein a percent of total number lesions that disappeared at the end of treatment compared to baseline is higher than on a surface having acne that is untreated. In an embodiment placebo is statistically better than no treatment. In an embodiment a placebo is better than no treatment or available treatments for acne.

In one or more embodiments there is provided a method for treating acne, including administering topically, to a surface having acne, a vehicle, wherein a percent of total number of non-inflammatory lesions that disappeared at the end of treatment compared to baseline is statistically higher than the number of inflammatory lesions that disappeared in the placebo group.

In one or more embodiments there is provided a method for treating acne, including administering topically, to a surface having acne, a vehicle, wherein at least about 40%, or at least about 50%, or at least about 52%, or at least about 54%, or at least about 56%, or at least about 58% or at least about 60%, or at least about 70%, or at least about 75% of total number lesions disappear after four weeks after the end of the treatment (F/U). In one or more embodiments these changes at F/U are statistically significant compared to baseline. In one or more embodiments these changes at F/U are statistically significant compared to 1% or 4% dose groups. In one or more embodiments the number of lesions at F/U is the same or similar compared to EOT. In one or more embodiments the number of lesions at F/U increases compared to EOT. In one or more embodiments there is the number of lesions at F/U decreases compared to EOT.

In one or more embodiments, there is provided an effective method for treating acne, as set out herein to patients with (i) more than twenty inflammatory but not more than fifty inflammatory lesions on the face (papules and/or pustules); (ii) at least twenty five and not more than 100 non-inflammatory lesions on the face (opened and/or closed comedones)(iii) no significant nodulocystic acne vulgaris on the face (less than 2 lesions) and (iv) receiving a score of at least Moderate on the Investigator's Global Assessment Scale.

In one or more embodiments there is provided a method for treating acne, including administering topically, to a surface having the acne, a vehicle, wherein the baseline severity of acne is at least moderate to severe, as judged by the number of acne lesions and investigator's global severity assessment (IGA).

In one or more embodiments there is provided a method for treating acne, including administering topically, to a surface having acne, a vehicle, wherein the mean number of lesions is at least 20 lesions.

In one or more other embodiments there is at least one lesion, or at least 5, or at least 10 or at least 15 lesions and in further embodiments there is at least 25, or at least 30 or at least 40 or at least 50 lesions.

In one or more embodiments there is provided a method for treating acne, including administering topically, to a surface having acne, a vehicle, wherein acne is low to moderate acne. In other embodiments the composition may be applied as aforesaid as a method of protecting the skin, for example, by preventing microbial infection or inflammatory acne

In one or more embodiments there is provided a method for treating acne, including administering topically, to a surface having acne, a vehicle, wherein the IGA score as assessed by the investigator at baseline is between 3.3-3.4, indicating moderate to severe acne at baseline. In other embodiments the composition may be applied to mild acne and in still further embodiments it may be applied to very severe acne.

Thus, it was unexpectedly demonstrated that the vehicle offered a safe and effective alternative to topical compositions containing for example retinoids and BPO for the topical treatment of acne vulgaris. The ease of use, with once daily dosing, as well as its broad spectrum of activity, early onset, the low level of skin irritation and adverse events and the rapid reduction in the number of lesions make it an attractive choice and a potentially valuable medication for the treatment of acute bacterial skin infections.

Further provided herein is a method of treating human skin disorders such as acne, rosacea, and/or impetigo by topical application of a vehicle foam or gel or liquid gel as described herein to a patient in need thereof.

The invention is described with reference to the following examples, in a non-limiting manner. The following examples exemplify the foamable compositions and methods described herein. The examples are for the purposes of illustration only and are not intended to be limiting. Many variations will suggest themselves and are within the full intended scope.

### Methods

### Canisters Filling and Crimping

Each aerosol canister is filled with the pre-foam formulation (“PFF”, i.e., foamable carrier) and crimped with valve using vacuum crimping machine. The process of applying a vacuum will cause most of the oxygen present to be eliminated. Addition of hydrocarbon propellant may, without being bound by any theory, further help to reduce the likelihood of any remaining oxygen reacting with an ingredient. It may do so, without being bound by any theory, by one or more of dissolving in, to the extent present, the oil or hydrophobic phase of the formulation, by competing with some oxygen from the formulation, by diluting out any oxygen, by a tendency of oxygen to occupy the dead space, and by oxygen occupying part of the space created by the vacuum being the unfilled volume of the canister or that remaining oxygen is rendered substantially ineffective in the formulation.

### Pressurizing & Propellant Filling

Pressurizing is carried out using a hydrocarbon gas or gas mixture. Canisters are filled and then warmed for 30 seconds in a warm bath at 50° C. and well shaken immediately thereafter.

### Tests

By way of non-limiting example the objectives are briefly set out below as would be appreciated by a person of skill in the art.

### Collapse Time

Collapse Time, which is the measure of thermal stability, is examined by dispensing a given quantity of foam and photographing sequentially its appearance with time during incubation at 36° C. The collapse time result is defined as the time when the foam height reaches 50% of its initial height or if the foam has not yet reached 50% of its initial height after say 180 seconds then the collapse time is recorded as being >180. By way of illustration, one foam may remain at 100% of its initial height for three minutes, a second foam may reach 90% of its initial height after three minutes, a third foam may reach 70% of its initial height after three minutes, and a fourth foam may reach 51% of its initial height after three minutes, nevertheless in each of these four cases the collapse time is recorded as >180 seconds since for practical purposes for easy application by a patient to a target the majority of the foam remains intact for more than 180 seconds. If the foam, for example, reaches 50% of its original height after say 100 seconds it would be recorded as having a collapse time of 100 seconds. It is useful for evaluating foam products, which maintain structural stability at skin temperature for at least 1 minute. Foams which are structurally stable on the skin for at least one minute are termed “short term stable” carriers or foams.

Alternatively, a Simple Collapse Time can be assessed by placing a foam sample on the warm fingers of a volunteer and measuring the time it takes to melt on the fingers.

### Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000CP, the viscosity at 1 RPM may be measured, although the figures are of a higher magnitude. Unless otherwise stated, viscosity of the pre-foam formulation (PFF) is provided. It is not practical to try and measure the viscosity of the foamable formulation with regular propellants since they have to be stored in sealed pressurized canisters or bottles. In order to simulate the viscosity in the foamable formulations with propellant an equivalent weight of pentane (a low volatile hydrocarbon) is added to and mixed with the pre-foam formulation and left overnight. The viscosity is then measured as above.

### FTC (Freeze Thaw Cycles)

Foam appearance under extreme conditions of repeated heating and cooling is evaluated by cycling through cooling, heating, (first cycle) cooling, heating (second cycle) etc., conditions, commencing with −10° C. (24 hours) followed by +40° C. (24 hours) and measuring the appearance following each cycle. The cycle is repeated up to three times.

### Microbiological Tests

Microbial load: Testing was performed according to EP 2.6.12 and 2.6.13 as described in the European Pharmacopeia.

Preservative efficacy: Testing was performed according to USP <51> and EP 5.6, 2007 5.1.3. as described in the European and US Pharmacopeia.

The test consists of challenging the product with specified microorganisms, storing the inoculated preparations at a prescribed temperature, removing the inoculated samples at specified intervals of time and counting the number of viable organisms in the withdrawn samples using a plate-count procedure. Formulations were challenged by introducing the following microorganisms:

Escherichia coli (ATCC no. 8739)
Staphylococcus aureus (ATCC no. 6538)
Pseudomonas aeruginosa (ATCC no. 9027)
Candida albicans (ATCC no. 10231)
Aspergillus niger (ATCC no. 16404)

The number of colony-forming units (cfu/g) determined at each incubation time point was compared to the number of cfu/g measured in non-inoculated control samples. In order to verify that the samples tested are free of microbial contaminants, the microbial load (base-line) in the samples was determined prior to preservative efficacy testing. Study results are expressed as the number of surviving microorganisms (cfu/g).

Water Activity (Aw): The test for water activity was performed on pre-foam formulation samples introduced into the measuring cell of a PAWKIT water activity meter from DECAGON.

In-vitro effect on microbial growth: The tested microorganism is grown on Tryptic Soy Agar Slants. After incubation, the bacteria is harvested using sterile buffer phosphate pH 7.0, to obtain a microbial count of about 10⁴ cfu/ml. 0.2 ml of the above suspension is spread on Letheen Agar plate and put aside to dry for 20 minutes at room temperature. A sterile disc of 6 mm diameter which has been soaked in 10 μl of the tested antibacterial pre-foam-formulation (PFF) is put on the microbial film, the plate is incubated at 35° C. for 1-2 days. A control experiment is also performed where no antibacterial material is put on the sterile discs. Antimicrobial activity of the tested material inhibits growth of the microorganism around the disc, leaving a transparent zone around it. The diameter of the inhibition zone is measured in mms.

Compatibility e.g. with minocycline: Excipient is incubated with active agent individually at one or more temperatures and at different ratios of active agent to a single excipient for a certain fixed period or to the point where degradation was suspected. The period can be for example 3 or 7 or 14 or 21 or 28 days or longer. Visual inspection is a criterion for indication of compatibility. Any change of color indicates oxidation or degradation. For example, the color of an intact minocycline HCL (MCH) suspension is a pale yellow; and a change of color e.g., to dark orange, red, green, brown and black, indicates oxidation or degradation. Tests are also carried out with combinations of excipients.

### EXAMPLES

### Example 1

### General Manufacturing Procedures for a Gel or a Foam

The following procedures were used to produce gel or foam samples, in which only the steps relevant to each formulation were performed depending on the type and nature of ingredients used.

Step 1: Hydrophobic solvents such as soybean oil, coconut oil, mineral oils are mixed at room temperature.

Step 2: The formulation is warmed to 85° C. and solid compounds such as fatty alcohols, fatty acids and waxes arc added and mixed until complete dissolution. Fumed silica and if present cosmetic agents and/or color agents are also added and mixed at this stage.

Step 3: The formulation is cooled down to 30-40° C. cyclomethicone and active agents such as tetracycline if present are added under mixing until formulation homogeneity is obtained.

Step 4: For gel compositions, the formulation is packaged in suitable containers. For foamable compositions, the formulation is packaged in aerosol canisters which are crimped with a valve, pressurized with propellant and equipped with an actuator suitable for foam dispensing. Optionally, a metered dosage unit is utilized, to achieved delivery of desirable and/or repeatable measured doses of foam.

Step 5: For foamable compositions, pressurizing is carried out using a hydrocarbon gas or gas mixture. Canisters are filled and then warmed for 2 minutes in a warm bath at 60° C. and well shaken immediately thereafter.

Step 6: The canisters or containers are labeled.

### Example 2

The following compositions used in the Clinical study, were prepared according to the manufacturing procedures detailed in Example 1.

TABLE 2a
Vehicle formulation

| | Formulation |
|---|---|
| | 244 placebo (vehicle) |
| Ingredients | % w/w |
| Light Mineral oil | 5.499 |
| Cyclomethicone | 5.00 |
| Coconut oil | 23.60 |
| Soybean oil | 50.00 |
| Hydrogenated castor oil | 2.00 |
| Beeswax | 2.00 |
| Myristyl alcohol | 2.50 |
| Cetostearyl alcohol | 3.50 |
| Stearyl alcohol | 1.50 |
| Behenyl alcohol | 1.10 |
| Fumed Silica (SiO2) | 0.25 |
| Stearic acid | 3.00 |
| Quinoline yellow WS | 0.05 |
| (D&C yellow 10) | |
| Quinoline yellow SS | 0.001 |
| (D&C yellow 11) | |
| Total | 100 |
| Propellant AP-70 | 12.00 |

TABLE 2b
Formulations of 1% Minocycline and 4% Minocycline

Formulations

| | 244B | 244A |
|---|---|---|
| | (1% Minocycline) | (4% Minocycline) |
| Ingredients | % w/w | % w/w |
| Light Mineral oil | 4.44 | 1.11 |
| Cyclomethicone | 5.00 | 5.00 |
| Coconut oil | 23.60 | 23.60 |
| Soybean oil | 50.00 | 50.00 |
| Hydrogenated castor oil | 2.00 | 2.00 |
| Beeswax | 2.00 | 2.00 |
| Myristyl alcohol | 2.50 | 2.50 |
| Cetostearyl alcohol | 3.50 | 3.50 |
| Stearyl alcohol | 1.50 | 1.50 |
| Behenyl alcohol | 1.10 | 1.10 |
| Fumed Silica (SiO2) | 0.25 | 0.25 |
| Stearic acid | 3.00 | 3.00 |
| Minocycline HCl | 1.11 | 4.44 |
| (90% potency) | | |
| Total | 100 | 100 |
| Propellant AP-70 | 12.00 | 12.00 |

All inactive ingredients used in the formulation are intended for topical use and listed in the current FDA Inactive Ingredient Database; concentrations used do not exceed the maximum concentrations given in Database.

The composition according to example 1a surprisingly shows beneficial properties.

### Example 3

### Clinical Study Phase II in Acne Vulgaris Patients

1. Study Synopsis

STUDY TITLE: A Pilot, Multicenter, Randomized, Double Blind, Placebo Controlled, Parallel Group, Dose Range Finding Study, to Evaluate the Tolerability and Safety of FXFM244 Antibiotic Foam and to Monitor its Clinical Effect in Acne Vulgaris Patients.

OBJECTIVES: (i) To assess the safety and tolerability of topical FXFM244 antibiotic foam in a population of acne vulgaris patients; (ii) To detect clinically significant efficacy of FXFM244 antibiotic foam for treatment of acne vulgaris and (iii) to establish a safe and effective dose.

STUDY MEDICATION: Minocycline hydrochloride foam (MCH 1% and MCH 4% compositions,) and placebo (vehicle foam) as set out Table 2a and Table 2b above.

DOSAGE: Patients were treated topically on the facial skin areas affected by acne vulgaris once daily for 12 weeks. The first dose was applied in the presence of the study Investigator or his assignee. Subsequent applications were made by the patient once daily at bedtime to the same region.

INDICATION: Acne vulgaris

DESIGN: 150 male or female patients, between ages of 12 to 25 years, diagnosed with moderate to severe acne vulgaris entered a randomized, double (investigator/patient) blind, placebo controlled, parallel group, dose range finding study. At the screening/baseline visit, after signing informed consent and undergoing screening procedures, eligible subjects were randomized in 1:1:1 ratio to one of three treatment groups receiving, 1% minocycline foam (FXFM244, 1%), 4% minocycline foam (FXFM244 4%) or Placebo. The first dose of study drug was applied topically in the presence of study investigator or designee to facial skin areas. Subsequently, subjects continued to self-apply study drug to the same region daily, in the evening, for 12 weeks. Subjects returned to the clinic for efficacy, safety and tolerability evaluations 3, 6, 9 and 12 weeks post baseline visit. A post-treatment follow-up visit took place 4 weeks after end of treatment.

Subjects received clear instructions on how to apply the study drug on the face area. During the 12 weeks treatment period, prior to each daily topical administration, subjects were instructed to cleanse the face area with mild or soapless, non-medicated cleanser, and not use any other acne medications or moisturizers. At each study visit, subjects were dispensed one aerosol container and were instructed to return it at the following visit to evaluate subject's compliance.

Patients were instructed to shake the can before use, dispense a small amount of foam and apply a thin layer of medication on the affected area once a day for 12 weeks. A post-treatment follow-up visit took place 4 weeks after end of treatment.

The inclusion criteria specified that patients should have at least 20 and not more than 50 inflammatory lesions on the face (papules and/or pustules), at least 25 and not more than 100 non-inflammatory lesions on the face (opened and/or closed comedones) (iii) no significant nodulocystic acne vulgaris on the face (5 2 lesions) and (iv) receiving a score of at least 3 (Moderate) on the Investigator's Global Assessment Scale.

VARIABLES: Clinical examination, safety, tolerability and efficacy parameters (lesion count, global assessment, global improvement assessment, patient global improvement assessment photographs).

Endpoints and Outcomes

Efficacy Outcomes

1. Lesion count, number, numerical change in lesion count and % change in lesion count (inflammatory, non-inflammatory and total) at visit 2 (three weeks), 3 (six weeks), 4 (nine weeks), 5 (twelve weeks), 6 (four weeks after end of treatment (EOT)) compared to baseline;

2. Percentage of subjects who had a decrease of more than 50%, more than 60%, more than 70% or more than 80% in lesions by study group at visit 2, 3, 4, 5, 6 compared to baseline; 3. Investigator Global Assessment (IGA) for acne severity: IGA at visits 2, 3, 4, 5 and 6.

The statistical difference in IGA at visits 2, 3, 4, 5 and 6.

Number and % of subjects that meet the success criterion of “clear” or “almost clear” (grades less 2) at visits 2, 3, 4, 5, and 6

Number and % of subjects that meet the success criterion of “improvement of 2 grades from the baseline” at visits 2, 3, 4, 5 and 6

4. Investigator's Global Improvement Assessment (aided by photographs) comparison from baseline to visit 5 (12 weeks of treatment);

5. Patient's Global Improvement Assessment comparison from baseline to visit 5 (12 weeks of treatment)

Safety Outcomes

Assessment of safety was conducted at all six visits using the following parameters adverse events (AEs), physical examination, vital signs (blood pressure, heart rate, and oral body temperature) and concomitant medications.

Tolerability Outcome

Clinical assessment of skin irritation (erythema, dryness, pigmentation, peeling and itching) using a scale of 0 to 3

Statistical Methods:

Tests applied are e.g. two-tailed, and a p-value of 5% or less is considered statistically significant. STATA version 12.0 for Windows was used. See detailed discussion of statistical methodology in section 4 below.

### 2. Clinical Study Design

The protocol and informed consent forms were approved by each clinical site's local Ethics Committee (EC) and the Israel Ministry of Health prior to study initiation. To be eligible for the study, the subject or the subject's parent or legal guardian were required to sign a written informed consent document and were willing and able to comply with the requirements of the protocol.

The exclusion criteria specified that subjects had to be between ages of 12 to 25 with clinical diagnosis of moderate to severe acne vulgaris with facial involvement having the main severity criteria consisting of:

With at least twenty but not more than fifty inflammatory lesions on the face (papules and/or pustules); at least twenty five and not more than 100 non-inflammatory lesions on the face (open blackhead and/or closed comedones whitehead), no significant nodulocystic acne vulgaris on the face (less than 2 lesions) and receiving a score of at least Moderate on the Investigator's Global Assessment Scale. Subjects were required not to have had any known medical conditions that, in the Investigator's opinion could interfere with study participation. Subject had to refrain from use of all other topical acne medications or antibiotics or moisturizers, new brands of make-up, creams, lotions, powders or any topical product other than the assigned treatment to the treatment area for the duration of the study. Women of childbearing potential had to use an acceptable form of birth control during the study. Use of oral contraceptives had to remain constant within 3 month prior to baseline and throughout the study.

The exclusion criteria specified that patients should have any one of the following (i) a disease selected form a group consisting Acne Conglobata, Acne Fulminas, secondary acne (chloracne, drug induced acne) or severe acne requiring systemic treatment; (ii) Presence of any facial skin condition that would interfere with the diagnosis or assessment of acne vulgaris (e.g. rosacea, dermatitis, psoriasis, squamous cell carcinoma, eczema, acneform eruptions caused by medications, steroid acne, steroid folliculitis, or bacterial folliculitis); (iii) Excessive facial hair (beards, sideburns, moustaches, etc.) that would interfere with diagnosis or assessment of acne vulgaris; (iv) Known or suspected hypersensitivity to minocycline or any of the excipients in the study drug. (iv) Use of concomitant medication prior to the study including:
a. Use within 6 month prior to baseline of topical retinoids, oral retinoids (Accutane®) or therapeutic vitamin A supplements of greater than 10,000 units/day (multivitamins are allowed).
b. Use of systemic steroids, systemic antibiotics, systemic treatment for Acne Vulgaris, systemic anti-inflammatory agents within 4 weeks prior to baseline.
c. Use of topical steroids, α-hydroxy/glycolic acid, benzoyl peroxide, topical antibiotics, topical treatment for Acne Vulgaris, topical anti-inflammatory agents within 2 weeks prior to baseline.
d. Use for less than 3 month prior to baseline of estrogens or change in oral contraceptives therapy within less than 3 month prior to baseline;
e. Use on the face of: cryodestruction or chemodestruction, dermabrasion, photodynamic therapy, acne surgery, intralesional steroids or X-ray therapy within 4 weeks prior to baseline.
f. Use of the following concomitant medications throughout the duration of the study (unless approved by the Investigator and medical monitor): topical antibiotics on the face, oral antibiotics, topical steroids on the face, oral steroids, topical anti-inflammatory drugs on the face, oral anti-inflammatory drugs, topical and/or oral drugs for Acne Vulgaris other than the study drug, topical retinoid drugs on the face, oral retinoid drugs or therapeutic vitamin A supplements of greater than 10,000 units/day, topical a hydroxyl/glycolic acid and/or benzoyl peroxide on the face, topical and/or Oral estrogens or any new oral contraceptives, spironolactone.
(v) Alcohol or drug abuse, according to assessment by the investigator; (vi) Known or suspected hypersensitivity to minocycline or any of the excipients in the Study Medication; (vii) Use of another investigational drug within 30 days prior baseline. (viii) Pregnant or lactating women; (ix) Use of tanning booths, sunbathing, or excessive exposure to the sun should be prohibited during the study; (x) Participation in clinical trial in the previous month prior to randomization.

Subjects were free to discontinue their participation in the study at any time and without prejudice to further treatment. The Investigator had to discontinue participation of any subject who requested to be withdrawn, or if it was determined that continuing in the study would result in a significant safety risk to the subject. The subject's participation in this study could have been discontinued due to any of the following reasons: interrupted therapy for >5 days, worsening of the disease, occurrence of a concomitant disease, intolerable adverse event, subject withdrew consent, relevant non-compliance with the protocol, investigator decided that withdrawal from the study was in the best interest of the subject, pregnancy, subject needed or used medication not permitted under the protocol.

Treatment was administered topically on facial skin areas affected with acne vulgaris once daily (OLD) at bedtime for 12 weeks. The mode of application was demonstrated by the investigator or study nurse at Visit 1 using a placebo from a demonstration kit that was supplied by the Applicants. Subjects were instructed to cleanse their face with a mild or soapless, non-medicated and then pat it dry. They were instructed to shake the canister before use, dispense a small amount of foam to a disposable plate and then apply a small amount of the foam using the tip of their finger for each area: forehead cheeks and chin. Subjects were instructed to treat the nose area only if affected and to apply the foam on the whole area, not just on visible lesions. Application was attained by collapsing and spreading it as a thin layer on the affected area. Patients were further instructed not to apply moisturizers, new brands of make-up, creams, lotions, powders or any topical product other than the assigned treatment to the treatment area. Patients were instructed to minimize exposure to sunlight, including sunlamps, while using the compositions. Use of sunscreen products over treated areas was recommended when sun exposure could not be avoided.

A total of a hundred and fifty eligible subjects (conforming to both inclusion and exclusion criteria) were enrolled and randomized into three parallel study groups, testing the two different strengths (minocycline 1% and 4%) of the study medication and placebo formulation (without minocycline) with fifty subjects in each treatment group. The study included six scheduled study visits: Day 1 (Visit 1-Baseline) screening and treatment initiation; Week 3 (±7)—(Visit 2 first interim visit); Week 6 (+7)—(Visit 3—second interim visit), Week 9 (+7)—(Visit 4—third interim visit), Week 12 (+5)—(Visit 5—End of Treatment (EOT)) and Week 16 (±7) (Visit 6—Follow-up (F/U)). Clinical assessments efficacy, safety and tolerability evaluations were done at Baseline, interim visits, EOT and F/U. At each visit, patients were evaluated by Investigator via lesion counts, clinical assessment of skin irritation, and a global assessment and improvement assessment. Any clinical adverse events was recorded. At Week 12, in addition to the above, the patient provided his/her Patient's Global Improvement Assessment and a satisfaction questionnaire regarding usability and treatment satisfaction (See Table 2.1 below).

TABLE 2.1
Study assessment table

| | | | | | | Follow- |
|---|---|---|---|---|---|---|
| | Treatment Period | | | | | Up |
| Study Assessment | Screening/Baseline | | | | | Period |
| Visit No. | 1 | 2 | 3 | 4 | 5 | 6 |
| Study Week | 0 | 3 | 6 | 9 | 12 | 16 |
| Visit window | 1 | ±7 | ±7 | ±7 | ±5 | ±7 |
| Informed consent | X | | | | | |
| Inclusion/exclusion criteria | X | | | | | |
| Demographic data | X | | | | | |
| Medical history | X | | | | | |
| Acne disease history | X | | | | | |
| Previous acne medications and | X | | | | | |
| response to these medications | | | | | | |
| Randomization | X | | | | | |
| Physical examination and vital signs | X¹ | X | X | X | X | X |
| Clinical assessment (skin | X | X | X | X | X | X |
| lesion count) | | | | | | |
| Investigator's Global Assessment | X | X | X | X | X | X |
| (IGA) of acne severity | | | | | | |
| Investigator's Global Improvement | | X | X | X | X | X |
| Assessment. | | | | | | |
| Photography of face area | X | X | X | X | X | X |
| Patient's Global Improvement | | | | | X | |
| Assessment | | | | | | |
| Tolerability (skin irritation) | X | X | X | X | X | X |
| assessment | | | | | | |
| Urine pregnancy test (for child- | X | X | | | | |
| bearing potential women)² | | | | | | |
| Adverse events | | X | X | X | X | X |
| Concomitant medication | X | X | X | X | X | X |
| Dispense study medication | X | X | X | X | | |
| Drug accountability and compliance | | X | X | X | X | |
| Dispense patient diary | X | | | | | |
| Collect patient diary | | | | | X | |

¹At screening/baseline, physical examination included height and weight, in addition to heart/lung and abdomen; vital signs included heart rate, blood pressure and temperature
²In case pregnancy was suspected during the study, urine pregnancy test was performed

Subject compliance was monitored using a treatment diary and was calculated as the percentage of number of days the subject actually administrated the study drug to the sum of days of study drug administration plus days with no study drug administration.

Drug compliance of patients (i.e., the amount of drug used by each subject) was calculated by weighing each container before and after use, and calculating the difference in the weight of containers before and after use, divided by the total number of days study drugs was used by each patient.

Efficacy was determined by the investigator at each visit by evaluating the change in the number of at lesion count (inflammatory/non inflammatory and total), % change in lesion count, Investigator's global assessment (IGA) for acne severity (assisted by scale). A global improvement assessment by the Investigator (assisted by photographs) and global improvement assessment by the patient which is a subjective assessment.

Tolerability was determined at each visit by assessment of skin irritation parameters. Safety was determined by assessment of adverse events, performing physical examination and checking vital signs at each visit, checking pregnancy potential and concomitant medications.

### 3. Patient Demographics

Patients who enrolled into the study were classified as follow:

Safety population (SAF) included all subjects who were randomized, dispensed study medications and attended at least one post-baseline visit with safety evaluation.

The intent-to-treat (ITT) population consists of all randomized patients who Safety population (SAF) included all subjects who were randomized, dispensed study medications and attended at least one post-baseline visit with safety evaluation.

The per protocol (PP) population includes all patients who completed 12 weeks of treatment with no major protocol deviations.

Modified per protocol (mPP) population included all PP population who have used a mean >0.08 g of study drug per day. The mPP population differs from the PP population by only one patient and the results of the statistical efficacy analysis of the PP and mPP are nearly the same.

The baseline demographic variables (age, gender, weight, height and BMI) of all subjects (ITT population) were comparable across the three study groups (1%, 4% minocycline foam and Placebo) (p>0.2). Overall average age was approximately 16.5 (range 12.3 to 25.0) and half of all participating subjects were men. Mean BMI was within normal range in all study groups (range 13.7 to 37.5).

Approximately one-sixth of the subjects in the ITT population had medical history events. Some had active diagnoses and others were receiving treatment for their condition. None had dermatological abnormalities other than acne. There were no notable differences between treatment groups.

All subjects with childbearing potential had a negative pregnancy test at Screening/Baseline.

All subjects had a history of acne vulgaris. In the ITT population the duration of acne vulgaris disease at time of enrollment ranged from 2-120 months, averaging approximately 35 months. There was no significant difference between the three study groups.

Study treatment compliance was evaluated in the PP population, in terms of subject compliance and drug compliance throughout the 12 weeks treatment period. There were no significant differences in subject compliance between study groups (p>0.40).

In the ITT population, the baseline severity of acne vulgaris was significantly higher than the minimum criteria. The mean lesion counts at baseline was about 34.5 (36.5, 33.5 and 33.6 in the 1%, 4% and Placebo groups, respectively) for inflammatory lesions (eligibility criterion: 20); and about 44.8 (46.2, 43.3, and 45.1 in 1%, 4%, and Placebo groups, respectively) for non-inflammatory lesions (eligibility criterion: 25). The IGA score as assessed by the investigator at baseline was 3.3-3.4, indicating moderate to severe acne at baseline. There were no statistically significant differences between the groups with regard to acne vulgaris severity. Similar baseline lesion counts were observed in the PP population and mPP population. (not shown).

A total of 150 subjects were randomized into 3 study groups (1%, 4% and Placebo), with 50 subjects in each group. Among all subjects, 139 subjects conformed to criteria for the ITT and safety (SAP) population: 46 in the 1% group, 47 in the 4% group and 46 in the Placebo group. Of these, 96 conformed to criteria for the PP population: 31 in 1% group, 36 in 4% group and 29 in the Placebo group.

TABLE 2.2
Disposition of subjects, all randomized subjects by study group

| | FXFM244 1% | | FXFM244 4% | | Placebo | | Total | |
|---|---|---|---|---|---|---|---|---|
| | N = 50 | | N = 50 | | N = 50 | | N = 150 | |
| | N | % | N | % | N | % | N | % |
| Subject Disposition | | | | | | | | |
| Subject screened | 50 | 100 | 50 | 100 | 50 | 100 | 150 | 100 |
| Subjects randomized | 50 | 100 | 50 | 100 | 50 | 100 | 150 | 100 |
| Subjects who completed | 31 | 62 | 36 | 72 | 29 | 58 | 96 | 64 |
| the study-per protocol | | | | | | | | |
| Subjects who did not complete | 19 | 38 | 14 | 28 | 21 | 42 | 54 | 36 |
| the study per protocol | | | | | | | | |
| Reason for discontinuation | | | | | | | | |
| Randomized but did not attend | 4 | 8 | 3 | 6 | 4 | 8 | 11 | 7 |
| to post baseline visit | | | | | | | | |
| Consent withdrawal | 4 | 8 | 0 | 0 | 4 | 8 | 8 | 5 |
| Lost to follow up (LFU) | 4 | 8 | 5 | 10 | 4 | 8 | 13 | 9 |
| Other (protocol violation) | 1 | 2 | 1 | 2 | 1 | 2 | 3 | 2 |
| Other (non-compliance) | 1 | 2 | 0 | 0 | 1 | 2 | 2 | 1 |
| Other (out of visit 5 window) | 5 | 10 | 5 | 10 | 7 | 14 | 17 | 11 |

TABLE 2.3
Summary of analysis populations by study group

| | FXFM244 1% | | FXFM244 4% | | Placebo | |
|---|---|---|---|---|---|---|
| | N = 50 | | N = 50 | | N = 50 | |
| Analysis Population | N | % | N | % | N | % |
| Safety Population(SAF)^{a} | 46 | 92 | 47 | 94 | 46 | 92 |
| ITT Population ^{b} | 46 | 92 | 47 | 94 | 46 | 92 |
| Per Protocol Population (PP) ^{c} | 31 | 62 | 36 | 72 | 29 | 58 |
| mPP Population ^{d} | 31 | 62 | 35 | 70 | 29 | 58 |

### 4. Statistical Methodology

All measured variables and derived parameters are tabulated by descriptive statistics. Descriptive statistics summary tables included sample size, absolute and relative frequency of categorical variables and sample size, arithmetic mean, standard deviation, median, minimum and maximum for means of continuous variables per group. All statistical tests are analyzed to a significance level of 0.05.

### Demographic and Baseline Characteristics Display

The planned sample size of 150 subjects was selected based on prior literature acne studies, to provide adequate information on the effect of an anti-acne topical drug. No formal sample size calculation was performed for this study. The demographic and baseline evaluation were done for the ITT population. For the demographic and baseline categorical variables (gender, physical examinations of heart, lungs and abdomen and medical history variables) numbers and percentages were calculated. Distributions for the categorical variables were compared and analyzed by the Chi square test. For the demographic and baseline continuous variable (age, height, weight, vital signs and disease duration) ranges, medians, means and standard deviations were calculated. The results between continuous variable were analyzed by ANOVA (Analysis of Variance) Bonferroni multiple comparison test (subgroups>2).

### Efficacy Analysis

The efficacy evaluations are done for the Intent to Treat (ITT), the Per Protocol (PP) and the modified (mPP) populations. The efficacy endpoints are: (a) the change in the number (primary endpoint) and percentage (secondary endpoint) of lesion counts (inflammatory, non-inflammatory and total) at post baseline visits compared to baseline and (b) Investigator's Global Assessment (co-primary endpoint).

For the lesion count variables ranges, medians, means and standard deviations are calculated. Test for normality was done by Shapiro-Wilk normality test. As the null hypothesis that the lesion counts variables are normally distributed is rejected, therefore the lesion counts results were compared and analyzed with non-parametric tests. The results between the lesion counts variables are compared and analyzed by the Kruskal-Wallis test (a non-parametric test for subgroups≧2).

Another co-primary endpoint is the evaluation of the Investigator Global Assessment (IGA) at each visit; IGA is based on a scale of 0 to 4, where 0=clear, 1=almost clear, 2=mild, 3=moderate, 4=severe. For the IGA variables ranges, medians, means and standard deviations are calculated. As the null hypothesis that the IGA assessment variables are normally distributed, is not rejected, therefore the IGA assessments results were compared and analyzed with parametric tests. The results between the IGA assessment variables are compared and analyzed by the ANOVA (Analysis of Variance) Bonferroni multiple comparison test (a parametric test for subgroups>2).

IGA success is evaluated as (a) the number and percentage of subjects who met the success criterion no.1 of “clear or almost clear” (grades less than 2) at each post baseline visit and (b) the number and percentage of subjects who met the success criteria no.2 “improvement of at least 2 grades from the baseline” at each post baseline visit. The last two IGA success criteria are categorical variables. The distributions for categorical variables are compared and analyzed by the Chi square test (a parametric test), or by Fisher-Irwin exact test (a non-parametric test).

Two secondary endpoints related to acne improvement assessment after 12 weeks of treatment compared to baseline are evaluated: (a) the Investigator's Global Improvement assessment, which is based on based on a scale of 1 to 5, where 1=Worsening, 2=No change, 3=Slight improvement, 4=Moderate improvement, 5=Excellent improvement and (b) Patient's Global Improvement Assessment which is based on a scale of 1 to 4, where 1=Worse than prior to study 2=Same as prior to study, 3=Slightly better than prior to study, 4=Much better than prior to study.

The investigator's Global Improvement assessment and Patient's Global Improvement Assessment are calculated and analyzed as categorical variables, numbers and percentages are calculated. The distributions for the physician's global improvement assessment and patient's global improvement assessment categorical variables are compared and analyzed by the Chi square test (a parametric test), or by Fisher-Irwin exact test (a non-parametric test for small numbers).

Subject compliance is calculated as the percentage of number of days the subject actually administrated the study drug to the sum of days of study drug administration plus days with no study drug administration.

Drug compliance is calculated as the average of study drug weight used per day (the difference of containers' weight before and after use divided by the total number of days study drug was used).

### Safety and Tolerability Analysis

The safety and the tolerability analysis are done for the safety/ITT population. The safety analysis evaluated AEs, vital signs and physical examination.

Adverse events (AEs) are coded by the CTCAE (version 4.0; equivalent to MedDRA version 12), SOC(System Organ Class), preferred term and grade. Incidences of AEs are presented by serious AEs (SAEs), severity (grade), relationship to study drug, duration, action taken and outcome of event. Intensity or severity of each adverse experience is characterized as follows: Mild=adverse experience which is easily tolerated; Moderate=Adverse experience sufficiently discomforting to interfere with daily activity; Severe=Adverse experience which prevents normal daily activities.

For the summary by severity, subjects who have multiple occurrences of the same AE are classified according to the worst reported severity of the AE. For the summary by relationship to study drug, subjects who have multiple occurrences of the same AE are classified according to the strongest reported relationship to study medication. The AE variables are categorical variables. For the AE categorical variables numbers and percentages are calculated. Distributions for categorical variables were compared and analyzed by the Chi square test (a parametric test), or by Fisher-Irwin exact test (a non-parametric test).

The tolerability variables (erythema, dryness, pigmentation, peeling and itching) used a scale of 0 to 3, where 0=None, 1=Mild 2=Moderate, 3=Severe. Tolerability assessment is evaluated as the most severe irritation score from all 5 parts of the face. The tolerability variables are calculated and compared as categorical variables and analyzed by the Chi square test (a parametric test), or by Fisher-Irwin exact test (a non-parametric test for small numbers).

### 5. Clinical Response to Treatment

The clinical response to treatment, (clinical success or clinical failure) is derived from an efficacy evaluation using the following methods and scales:

### A. Number of Inflammatory and Non-Inflammatory Acne Lesions on the Face

At each study visit, the whole face area was assessed for number of acne lesions, change from base line and percent change from baseline in the cumulative number of lesions of each type inflammatory (papules, pustules and nodulocystic lesions) and non-inflammatory (open (blackhead) and closed (whitehead) comedones) as well total lesion counts (inflammatory plus non-inflammatory lesions). B. Investigator Global Assessment—Current Severity Assessment of Acne Sign/Symptom

The severity of each of the following signs/symptoms was assessed and measured at baseline and at all follow up visits using scale described above (see section 4—Statistical Analysis (efficacy analysis). In addition acne exacerbation was evaluated and any change in the severity of the disease was described by the investigator.

### C. Investigator's Global Improvement Assessment

This score was determined by the investigator and represents the patient's overall change from baseline in the severity of acne. The change in the patient's condition was scored at each post baseline visit compared to baseline using the scale described above (see section 4—Statistical Analysis efficacy analysis). The patient was photographed at baseline to facilitate comparisons at each post-baseline visit.

### D. Patient's Global Improvement Assessment

This score was obtained from the patient only at the end of treatment visit (visit 5 week 12). It results from the question “Considering your acne just before starting treatment and considering your condition today, indicate the change you have experienced according to the scale below”. The scale is provided in section 4 Statistical Analysis (efficacy assessment) above.

### E. Photography

Photographs were taken at each study visit, for documentation purpose, using a digital camera always under the same conditions: front, left side and right side of the face. This aided in estimating lesion area, severity and clinical response. Photographs are not shown herein.

### 6. Safety and Tolerability

Safety and tolerability were determined for all randomized patients by the investigator at each visit. Safety was assessed using different parameters such as vital signs (blood pressure, heart rate, temperature), physical examination of body systems (heart, lungs, abdomen and, where appropriate, other body systems as indicated), pregnancy potential. Adverse events and concomitant medications. Intensity or severity of each adverse experience was characterized using the scale described above (see Section 4—Safety and tolerability assessment).

Significant findings present prior to the start of treatment were included in the relevant medical history CRF. Significant findings made after the start of study drug (or therapy) which met the definition of an adverse events were recorded on the adverse event CRF. Height and weight measurements were recorded at visit 1 (screening/baseline). Any medication or treatment administered during the study as well as changes in established dosages of concomitant medication were documented in the CRFs.

Women of childbearing potential underwent urine pregnancy test at visit 1 (Screening/Baseline). If at subsequent visits, pregnancy was suspected, urine pregnancy test was to be performed.

Tolerability was determined by clinical assessment of skin irritation using different parameters such as, erythema, dryness, pigmentation, peeling and itching at each study visit and at follow up. Based on patient subjective assessment, itching was recorded.

### 7. Satisfaction

At study visit 5 (EOT), a questionnaire was filled out by subjects and the subjects' parents regarding usability and treatment satisfaction. Different parameters were assessed after treatment such as greasiness, shininess, stickiness, moistness of the skin, general feeling, odor, use of pump and control of the amount, general satisfaction from foam and recommendation.

### 8. Study Results

### 8.1. Study Population

The study was conducted at three centers. A total of a hundred and fifty patients clinically diagnosed with at least moderate acne vulgaris were randomized into three groups, with fifty patients in each group. One group received the 1% minocycline foam the second group received the 4% minocycline foam and the third group received the placebo vehicle described in Example 2.

The study was double-blinded and neither the investigators, nor the patients and their parents nor their legal guardian, nor the Applicants knew what strength of medication was dispensed or if the patient received placebo. A dye was introduced in the placebo formulation so that the placebo foam looked like the foam containing minocycline.

### 8.2. Efficacy on Inflammatory Lesions

The effect of the investigated products on inflammatory lesions is detailed in tables 8.2.1, 8.2.2 and 8.2.3 below. The study enrolled patients having moderate severe to severe acne: the mean number of inflammatory lesions was higher than that in many of the studies conducted on other acne-related drugs. The mean lesion counts at baseline was about 34.5 for inflammatory lesions whereas the eligibility criterion was 20. Clinical assessment of acne lesions at baseline indicated no significant differences in lesion counts between groups (mean total ˜80 lesions, including mean ˜37 inflammatory lesions in subjects receiving the 1% minocycline foam, and ˜34 in the subjects receiving the 4% minocycline foam and Placebo). Nevertheless, a substantial decrease in the number of inflammatory lesions was observed in all groups.

TABLE 8.2.1
Mean number of inflammatory lesions over 16
weeks for each treatment (count by visit).

| Lesion Numbers | Weeks | | | | | |
|---|---|---|---|---|---|---|
| (mean) | 0 | 3 | 6 | 9 | 12 | 16 |
| Placebo | 33.6 | 21.1 | 17.8 | 16.8 | 17.2 | 16.0 |
| MCH 1% | 36.5 | 23.2 | 15.3 | 14.9 | 13.1 | 13.3 |
| MCH 4% | 33.5 | 16.6 | 10.4** | 9.3** | 9.8** | 9.7** |

**Statistically different from placebo (p < 0.005, ITT population).

TABLE 8.2.2
Mean change from baseline in the number of inflammatory
lesions over 16 weeks for each treatment.

Mean Lesion

| Number Change | Weeks | | | | |
|---|---|---|---|---|---|
| from Baseline | 3 | 6 | 9 | 12 | 16 |
| Placebo | −12.5 | −15.8 | −16.8 | −16.5 | −17.6 |
| MCH 1% | −13.3 | −21.2** | −21.6** | −23.4** | −23.2** |
| MCH 4% | −16.9** | −23.2** | −24.2** | −23.7** | −23.9** |

**Statistically different from placebo (p < 0.05) for ITT population.

TABLE 8.2.3
Mean percentage of decrease from baseline in the number
of inflammatory lesions over 16 weeks for each treatment.

% Number

| Change from | Weeks | | | | |
|---|---|---|---|---|---|
| Baseline | 3 | 6 | 9 | 12 | 16 |
| Placebo | −40% | −50% | −52% | −51% | −54% |
| MCH 1% | −39% | −59% | −61% | −67%** | −65% |
| MCH 4% | −53%** | −71%** | −74%** | −72%** | −73%** |

**Statistically different from placebo (p < 0.05, ITT population

TABLE 8.2.4
Portion of subjects with 50%, 60%, 70%, or 80% reduction in inflammatory
lesions by study group and visit (ITT Population)

| | | | FXFM244 1% | | FXFM244 4% | | Placebo | | P value |
|---|---|---|---|---|---|---|---|---|---|
| | | | N = 46 | | N = 47 | | N = 46 | | (FXFM244 4% |
| | | | N | % | N | % | N | % | vs. Placebo) |
| More | 6 | weeks | 35 | 76.1 | 37 | 78.7 | 24 | 52.2 | 0.0070 |
| than 50% | 9 | weeks | 33 | 71.7 | 39 | 83.0 | 26 | 56.5 | 0.0050 |
| reduction | 12 | weeks | 36 | 78.3 | 40 | 85.1 | 26 | 56.5 | 0.0020 |
| | 16 | weeks | 34 | 73.9 | 39 | 83.0 | 28 | 60.9 | 0.0180 |
| More | 6 | weeks | 26 | 56.5 | 34 | 72.3 | 19 | 41.3 | 0.0030 |
| than 60% | 9 | weeks | 26 | 56.5 | 39 | 83.0 | 21 | 45.7 | 0.0001 |
| reduction | 12 | weeks | 32 | 69.6 | 40 | 85.1 | 23 | 50.0 | 0.0001 |
| | 16 | weeks | 31 | 67.4 | 37 | 78.7 | 26 | 56.5 | 0.0220 |
| More | 6 | weeks | 21 | 45.7 | 30 | 63.8 | 14 | 30.4 | 0.0010 |
| than 70% | 9 | weeks | 19 | 41.3 | 32 | 68.1 | 16 | 34.8 | 0.0010 |
| reduction | 12 | weeks | 25 | 54.4 | 33 | 70.2 | 13 | 28.3 | 0.0001 |
| | 16 | weeks | 24 | 52.2 | 35 | 74.5 | 16 | 34.8 | 0.0001 |
| More | 6 | weeks | 10 | 21.7 | 20 | 42.6 | 10 | 21.7 | 0.0320 |
| than 80% | 9 | weeks | 10 | 21.7 | 21 | 44.7 | 8 | 17.4 | 0.0050 |
| reduction | 12 | weeks | 15 | 32.6 | 22 | 46.8 | 7 | 15.2 | 0.0010 |
| | 16 | weeks | 16 | 34.8 | 28 | 59.6 | 13 | 28.3 | 0.0020 |

The above results show that patients who received a foam vehicle had a 50% or more decrease in the number of inflammatory lesions starting from week 6, which is better that the results obtained by oral treatments with minocycline (44% decrease at week 12), according to the information published on the prescription leaflets of commercially available oral minocycline treatments provided below. Surprisingly, the placebo had a marked positive effect on inflammatory lesions.

The following conclusions may be derived from Table 8.2.1-8.2.3. The vehicle which functions as the carrier of 1% and 4% MCH compositions greatly contributes to the decrease in the number of inflammatory lesions.

Lesion counts in all three groups decreased as compared to baseline already at week 3 and throughout the study.

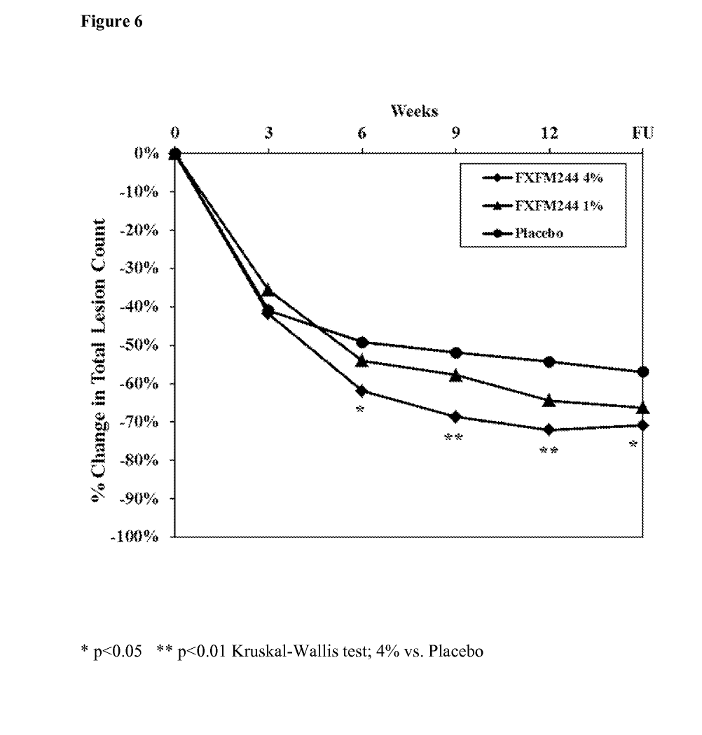

The effect of the vehicle on reducing the number of inflammatory lesions was about 50% (from week 6) which is relatively high (Table 8.2.3). This reduction was demonstrated in more than a half of the subjects receiving placebo (Table 8.2.4). The percent reduction in the number of inflammatory lesion in subjects receiving placebo started to flatten out after 6 weeks of treatment. These effects continued up to the follow-up visit. (see FIG. 6)

Without being bound by any theory, this can be attributed inter alia the (i) whole composition, and/or (ii) the oils that extract the sebaceous matter from the gland, and/or (iii) the fatty alcohols and fatty acids, (iv) and/or the waxes.

A dose dependent effect was observed i.e. 4% was more effective than 1%, which in turn was more effective than placebo formulation, which in turn was more effective than no treatment.

### 8.3. Efficacy on Non-Inflammatory Lesions

The effect of the investigated products on non-inflammatory lesions is detailed in tables 8.3.1-8.3.5 below. As can be seen from the mean number non-inflammatory lesions at baseline, this study enrolled patients having acne of high severity: the mean number of lesions is higher that in many of the studies conducted on other acne-related drugs. The mean lesion counts at baseline was about 44.8 for non-inflammatory lesions whereas eligibility criterion was 25. Nevertheless, a substantial decrease in the number of non-inflammatory lesions was observed in all groups.

TABLE 8.3.1
Mean number of non-inflammatory lesions
over 16 weeks for each treatment.

| Lesion Numbers | Weeks | | | | | |
|---|---|---|---|---|---|---|
| (mean) | 0 | 3 | 6 | 9 | 12 | 16 |
| Placebo | 45.1 | 27.2 | 23.5 | 22.3 | 19.7 | 18.7 |
| MCH 1% | 46.2 | 32.4 | 24.7 | 22.0 | 18.7 | 16.7 |
| MCH 4% | 43.3 | 28.3 | 19.8 | 15.1 | 12.0** | 13.2 |

**Statistically different from placebo (p < 0.05, ITT population)

TABLE 8.3.2
Mean change from baseline in the number of non-inflammatory
lesions over 16 weeks for each treatment.

Mean Lesion

| | |
|---|---|
| Number Change | Weeks |

| from Baseline | 3 | 6 | 9 | 12 | 16 |
|---|---|---|---|---|---|
| Placebo | −17.9 | −21.6 | −22.8 | −25.4 | −26.4 |
| MCH 1% | −13.8 | −21.5 | −24.2 | −27.4 | −29.5 |
| MCH 4% | −15 | −23.5 | −28.2 | −31.4** | −30.1 |

**Statistically different from placebo (p ≦ 0.05)

TABLE 8.3.3
Mean percentage of decrease from baseline in the number of
non-inflammatory lesions over 16 weeks for each treatment.

Mean % number

| Change from | Weeks | | | | |
|---|---|---|---|---|---|
| Baseline | 3 | 6 | 9 | 12 | 16 |
| Placebo | −42% | −49% | −52% | −57% | −59% |
| MCH 1% | −34% | −51% | −56% | −65% | −68% |
| MCH 4% | −33% | −55% | −65% | −73%** | −69% |

**Statistically different from placebo (p < 0.05).

TABLE 8.3.4
Percent (%) of subjects with more than 50% reduction in
non-inflammatory lesion count from baseline by study group and visit

| | % of Subjects with >50% Reduction | | | | | | |
|---|---|---|---|---|---|---|---|
| | in Non-Inflammatory Lesion Count | | | | | | |
| | FXFM244 1% | | FXFM244 4% | | Placebo | | |
| | N = 46 | | N = 47 | | N = 46 | | |
| Visit no. | N | % | N | % | N | % | P value |
| Visit 3 | 27 | 58.7 | 32 | 68.1 | 25 | 54.4 | a: 0.3830 |
| (6 weeks) | | | | | | | b: 0.1740 |
| | | | | | | | c: 0.6740 |
| | | | | | | | d: 0.3470 |
| Visit 4 | 29 | 63.0 | 35 | 74.5 | 26 | 56.5 | a: 0.1860 |
| (9 weeks) | | | | | | | b: 0.0690 |
| | | | | | | | c: 0.5240 |
| | | | | | | | d: 0.2340 |
| Visit 5 | 35 | 76.1 | 42 | 89.4 | 33 | 71.7 | a: 0.0870 |
| (12 weeks) | | | | | | | b: 0.0380* |
| | | | | | | | c: 0.6350 |
| | | | | | | | d: 0.1060 |
| Visit 6 | 39 | 84.8 | 40 | 85.1 | 33 | 71.7 | a: 0.1800 |
| (FU) | | | | | | | b: 0.1170 |
| | | | | | | | c: 0.1290 |
| | | | | | | | d: 1.0000 |

*p ≦ 0.05 Sig; p > 0.05 NS;
a: FXFM244 4% vs FXFM244 1% vs placebo
b: FXFM244 4% vs placebo;
c: FXFM244 1% vs placebo;
d: FXFM244 4% vs FXFM244 1%

TABLE 8.3.5
Percent (%) of subjects with more than 70% reduction in
non-inflammatory lesion count from baseline by study group and visit

| | % of Subjects with >70% Reduction | | | | | | |
|---|---|---|---|---|---|---|---|
| | in Non-Inflammatory Lesion Count | | | | | | |
| | FXFM244 1% | | FXFM244 4% | | Placebo | | |
| | N = 46 | | N = 47 | | N = 46 | | |
| Visit no. | N | % | N | % | N | % | P value |
| Visit 3 | 16 | 34.8 | 18 | 38.3 | 18 | 39.1 | a: 0.9010 |
| (6 weeks) | | | | | | | b: 0.9340 |
| | | | | | | | c: 0.6660 |
| | | | | | | | d: 0.7250 |
| Visit 4 | 19 | 41.3 | 23 | 48.9 | 18 | 39.1 | a: 0.6040 |
| (9 weeks) | | | | | | | b: 0.3410 |
| | | | | | | | c: 0.8320 |
| | | | | | | | d: 0.4600 |
| Visit 5 | 27 | 58.7 | 27 | 57.5 | 17 | 37.0 | a: 0.0640 |
| (12 weeks) | | | | | | | b: 0.0480* |
| | | | | | | | c: 0.0370* |
| | | | | | | | d: 0.9030 |
| Visit 6 | 29 | 63.0 | 29 | 61.7 | 21 | 45.7 | a: 0.1720 |
| (FU) | | | | | | | b: 0.1210 |
| | | | | | | | c: 0.0940 |
| | | | | | | | d: 0.8940 |

*p ≦ 0.05 Sig; p > 0.05 NS;
a: FXFM244 4% vs FXFM244 1% vs placebo
b: FXFM244 4% vs placebo;
c: FXFM244 1% vs placebo;
d: FXFM244 4% vs FXFM244 1%

The above results show that patients who received vehicle had about a 50% or more decrease in the number of non-inflammatory lesions starting from week 6, reaching almost a 60% decrease at week 12 which continued also 4 weeks after the end of treatment, which is better that the results obtained by oral treatments with minocycline (no improvement at week 12), according to the information published on the prescription leaflets of commercially available oral minocycline treatments. Surprisingly, the placebo had a marked positive effect on non-inflammatory lesions which was also higher than other available topical treatments (see Table 8.8.1). A decrease of more than 50% of the non-inflammatory lesions was demonstrated in over 70% of the subjects treated with placebo at 12 weeks (Table 8.3.4).

The following conclusions may be derived from Table 8.3.1-8.3.5 regarding non-inflammatory lesions.

A dose dependent effect was observed. The effect of the vehicle was between about 50% and about 60% which is relatively high. Without being bound by any theory, this can be attributed inter alia the (i) whole composition, and/or (ii) the oils that extract the sebaceous matter from the gland, and/or (iii) the fatty alcohols and fatty acids, (iv) and/or the waxes.

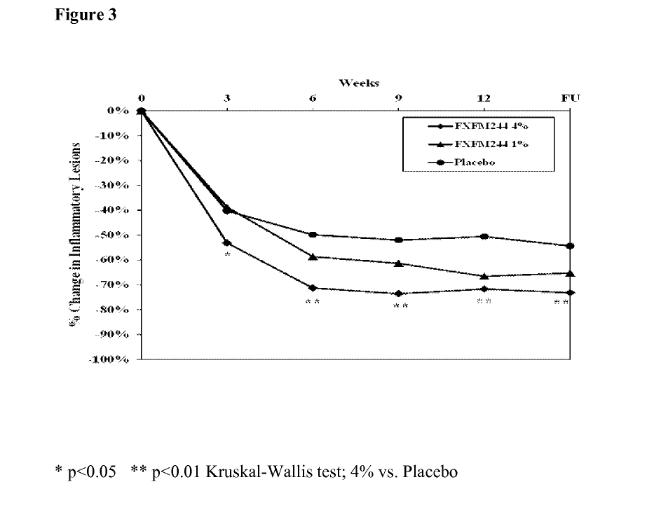

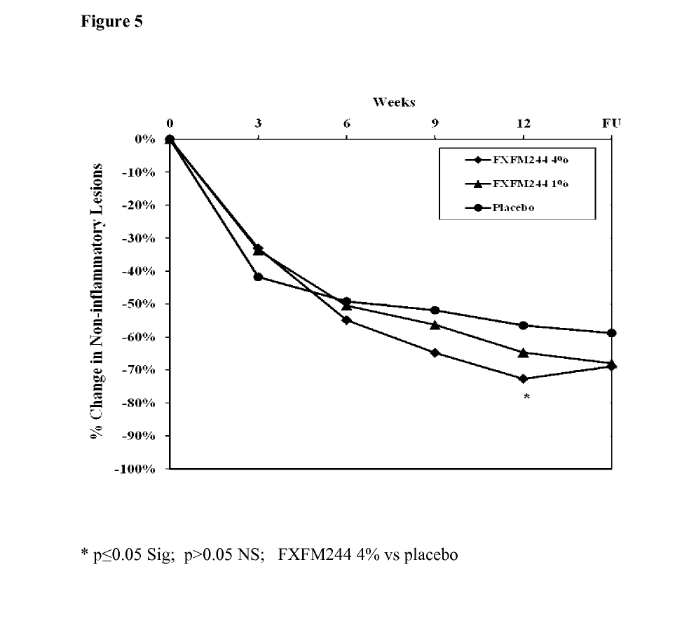

The placebo effect was more apparent at twelve weeks with non-inflammatory lesions (57%) compared to inflammatory lesions (51%). However, in both types of lesions the effect was apparent starting from week 3 (see e.g. FIG. 3 and FIG. 5).

### 8.4. Efficacy on Total Lesions

The effect of the investigated products on total lesions (inflammatory and non-inflammatory) is detailed in tables 8.4.1-8.4.6 below. As can be seen from the mean number total lesions at baseline, this study enrolled patients having moderate severe to severe acne: the mean number of total lesions (80) is higher than that seen in many of the studies conducted on other acne-related drugs. Nevertheless, a substantial decrease in the number of total lesions was observed in all groups.

TABLE 8.4.1
Mean number of total lesions over 16 weeks for each treatment.

| Lesion Numbers | Weeks | | | | | |
|---|---|---|---|---|---|---|
| (Mean) | 0 | 3 | 6 | 9 | 12 | 16 |
| Placebo | 78.7 | 48.3 | 41.3 | 39.1 | 36.9 | 34.7 |
| MCH 1% | 82.7 | 55.6 | 40.0 | 37.0 | 31.8 | 30.0 |
| MCH 4% | 76.9 | 44.7 | 30.1** | 23.9** | 21.7** | 23.2** |

**Statistically different from placebo (p < 0.05).

TABLE 8.4.2
Mean change from baseline in the number of total
lesions over 16 weeks for each treatment.

Lesion Number

| | |
|---|---|
| Change from | Weeks |

| Baseline | 3 | 6 | 9 | 12 | 16 |
|---|---|---|---|---|---|
| Placebo | −30.4 | −37.4 | −39.6 | −41.9 | −44 |
| MCH 1% | −27.1 | −42.7 | −45.7 | −50.8 | −52.7 |
| MCH 4% | −32.2 | −46.8 | −52.4** | −55.1** | −53.7 |

**Statistically different from placebo (p < 0.05) ITT.

TABLE 8.4.3
Mean percentage of decrease from baseline in the number
of total lesions over 16 weeks for each treatment.

Mean % Number

| Change from | Weeks | | | | |
|---|---|---|---|---|---|
| Baseline | 3 | 6 | 9 | 12 | 16 |
| Placebo | −41% | −49% | −52% | −54% | −57% |
| MCH 1% | −36% | −54% | −58% | −64% | −66% |
| MCH 4% | −42% | −62%** | −69%** | −72%** | −71%** |

**Statistically different from placebo (p < 0.05).

TABLE 8.4.4
Percent (%) of subjects who had more than 50% reduction in total
lesion count from baseline by study group and visit (ITT/LOCF)

| | % of Subjects with >50% Reduction | | | | | | |
|---|---|---|---|---|---|---|---|
| | in Total Lesion Count | | | | | | |
| | FXFM244 1% | | FXFM244 4% | | Placebo | | |
| | N = 46 | | N = 47 | | N = 46 | | |
| Visit no. | N | % | N | % | N | % | P value |
| Visit 3 | 31 | 67.4 | 32 | 68.1 | 28 | 60.9 | a: 0.7230 |
| (6 weeks) | | | | | | | b: 0.4670 |
| | | | | | | | c: 0.5140 |
| | | | | | | | d: 0.9430 |
| Visit 4 | 30 | 65.2 | 38 | 80.9 | 27 | 58.7 | a: 0.0610 |
| (9 weeks) | | | | | | | b: 0.0200* |
| | | | | | | | c: 0.5190 |
| | | | | | | | d: 0.0890 |
| Visit 5 | 32 | 69.6 | 42 | 89.4 | 30 | 65.2 | a: 0.0120* |
| (12 weeks) | | | | | | | b: 0.0070** |
| | | | | | | | c: 0.6560 |
| | | | | | | | d: 0.0220* |
| Visit 6 | 36 | 78.3 | 38 | 80.9 | 30 | 65.2 | a: 0.1780 |
| (FU) | | | | | | | b: 0.0890 |
| | | | | | | | c: 0.1650 |
| | | | | | | | d: 0.7570 |

*p ≦ 0.05; Sig; p > 0.05 NS;
a: FXFM244 4% vs FXFM244 1% vs placebo
b: FXFM244 4% vs placebo;
c: FXFM244 1% vs placebo;
d: FXFM244 4% vs FXFM244 1%

TABLE 8.4.5
Percent (%) of subjects with more than 60% reduction in
total lesion count from baseline by study group and visit

| | % of Subjects with >60% Reduction | | | | | | |
|---|---|---|---|---|---|---|---|
| | in Total Lesion Count | | | | | | |
| | FXFM244 1% | | FXFM244 4% | | Placebo | | |
| | N = 46 | | N = 47 | | N = 46 | | |
| Visit no. | N | % | N | % | N | % | P value |
| Visit 3 | 21 | 45.7 | 28 | 59.6 | 20 | 43.5 | a: 0.2410 |
| (6 weeks) | | | | | | | b: 0.1200 |
| | | | | | | | c: 0.8340 |
| | | | | | | | d: 0.1790 |
| Visit 4 | 23 | 50.0 | 36 | 76.6 | 21 | 45.7 | a: 0.0050** |
| (9 weeks) | | | | | | | b: 0.0020** |
| | | | | | | | c: 0.6760 |
| | | | | | | | d: 0.0080** |
| Visit 5 | 31 | 67.4 | 38 | 80.9 | 26 | 56.5 | a: 0.0410* |
| (12 weeks) | | | | | | | b: 0.0110* |
| | | | | | | | c: 0.2830 |
| | | | | | | | d: 0.1380 |
| Visit 6 | 33 | 71.7 | 34 | 72.3 | 27 | 58.7 | a: 0.2850 |
| (FU) | | | | | | | b: 0.1660 |
| | | | | | | | c: 0.1890 |
| | | | | | | | d: 0.9480 |

**p ≦ 0.01 Sig;
*p ≦ 0.05 Sig; p > 0.05 NS;
a: FXFM244 4% vs FXFM244 1% vs placebo
b: FXFM244 4% vs placebo;
c: FXFM244 1% vs placebo;
d: FXFM244 4% vs FXFM244 1%

TABLE 8.4.6
Percent (%) of subjects with more than 70% reduction in total lesion
count from baseline by study group and visit (ITT/LOCF)

| % of Subjects with | FXFM244 | | FXFM244 | | | | |
|---|---|---|---|---|---|---|---|
| >70% Reduction in | 1% | | 4% | | Placebo | | |
| Total Lesion Count | N = 46 | | N = 47 | | N = 46 | | |
| Visit no. | N | % | N | % | N | % | P value |
| Visit 3 (6 weeks) | 16 | 34.8 | 22 | 46.8 | 12 | 26.1 | a: 0.1120 |
| | | | | | | | b: 0.0380* |
| | | | | | | | c: 0.3650 |
| | | | | | | | d: 0.2380 |
| Visit 4 (9 weeks) | 19 | 41.3 | 26 | 55.3 | 18 | 39.1 | a: 0.2340 |
| | | | | | | | b: 0.1180 |
| | | | | | | | c: 0.8320 |
| | | | | | | | d: 0.1760 |
| Visit 5 (12 weeks) | 26 | 56.5 | 28 | 59.6 | 14 | 30.4 | a: 0.0090** |
| | | | | | | | b: 0.0050** |
| | | | | | | | c: 0.0120* |
| | | | | | | | d: 0.7650 |
| Visit 6 (FU) | 24 | 52.2 | 28 | 59.6 | 18 | 39.1 | a: 0.1370 |
| | | | | | | | b: 0.0490* |
| | | | | | | | c: 0.2090 |
| | | | | | | | d: 0.4720 |

*p ≦ 0.05 Sig;
p > 0.05 NS;
a: FXFM244 4% vs FXFM244 1% vs placebo
b: FXFM244 4% vs placebo;
c: FXFM244 1% vs placebo;
d: FXFM244 4% vs FXFM244 1%

The above results show that patients who received a vehicle foam had about a 50% or more mean decrease in the number of total lesions starting from week 6, reaching almost a 60% decrease at week 16 (see Table 8.4.3 and FIG. 6). A decrease of at least 50% in the total number of lesions was demonstrated in 65% of the subjects receiving placebo at week 12 onwards (Table 8.4.4). Surprisingly, the vehicle had a marked positive effect on the decrease in the total number lesions.

### 8.5. Efficacy According to Investigator's Global Assessment

The effect of the investigated products on total lesions is detailed in tables 8.5.1, 8.5.2, 8.5.3, 8.5.4, 8.5.5, 8.5.6 and 8.5.7 below. As can be seen from the mean Investigator's Global Assessment, this study enrolled patients having acne of high severity: 3.3-3.4 out of 4 grades.

TABLE 8.5.1
Mean IGA over 16 weeks for each treatment.

Weeks

| Mean IGA | 0 | 3 | 6 | 9 | 12 | 16 |
|---|---|---|---|---|---|---|
| Placebo | 3.4 | 2.7 | 2.5 | 2.5 | 2.4 | 2.3 |
| MCH 1% | 3.4 | 2.8 | 2.5 | 2.3 | 2.1 | 2.1 |
| MCH 4% | 3.3 | 2.6 | 2** | 1.9** | 1.7** | 1.8** |

**IGA score was statistically significantly lower than the placebo (p < 0.05, ITT population).

TABLE 8.5.2
Mean change from baseline in the IGA over 16 weeks for each treatment.

| Mean IGA Score | Weeks | | | | |
|---|---|---|---|---|---|
| Change from Baseline | 3 | 6 | 9 | 12 | 16 |
| Placebo | −0.6 | −0.9 | −0.9 | −1 | −1.1 |
| MCH 1% | −0.6 | −0.9 | −1.1 | −1.3 | −1.3 |
| MCH 4% | −0.7 | −1.3** | −1.4** | −1.7** | −1.5** |

TABLE 8.5.3
Mean percentage of decrease from baseline in the IGA
over 16 weeks for each treatment.

| Mean % IGA Score | Weeks | | | | |
|---|---|---|---|---|---|
| Change from Baseline | 3 | 6 | 9 | 12 | 16 |
| Placebo | −21% | −26% | −26% | −29% | −32% |
| MCH 1% | −18% | −26% | −32% | −38% | −38% |
| MCH 4% | −17% | −33% | −33% | −50% | −50% |

TABLE 8.5.4
Percentage of patients having an “almost clear” or “clear” IGA
(less than IGA = 2) over 16 weeks for each treatment.
% of patients with IGA “almost clear” or “clear” (less than IGA = 2)

| | 3 | 6 | 9 | 12 | 16 |
|---|---|---|---|---|---|
| Placebo | 4% | 21% | 26% | 20% | 33% |
| MCH 1% | 2% | 15% | 22% | 37% | 41% |
| MCH 4% | 9% | 38% | 49%*** | 53%*** | 55%*** |

***Statistically different from placebo (p < 0.05, ITT, PP).

TABLE 8.5.5
Percentage of patients having an IGA change of at least
2 units over 16 weeks fur each treatment.
% of patients with IGA change of at least 2 units

| | 3 | 6 | 9 | 12 | 16 |
|---|---|---|---|---|---|
| Placebo | 7% | 9% | 17% | 15% | 22% |
| MCH 1% | 4% | 7% | 11% | 22% | 22% |
| MCH 4% | 9% | 23% | 30% | 36%** | 34% |

**Statistically different from placebo (p < 0.021). Statistical Analysis was not performed at other time points.

TABLE 8.5.6
Investigator's global improvement assessment after 12 weeks
of treatment by study group (ITT Population)

| | FXFM244 | | FXFM244 | | | | |
|---|---|---|---|---|---|---|---|
| Investigator's | 1% | | 4% | | Placebo | | |
| assessment | N = 46 | | N = 47 | | N = 46 | | |
| 12 weeks | n | % | n | % | n | % | P value |
| Excellent | 24 | 52.2 | 30 | 63.8 | 15 | 32.6 | a: 0.0020** |
| Moderate | 17 | 37.0 | 12 | 25.5 | 13 | 28.3 | b: 0.0030** |
| Slight | 2 | 4.3 | 3 | 6.4 | 13 | 28.3 | c: 0.0040** |
| improvement | 3 | 6.5 | 2 | 4.3 | 2 | 4.3 | d: 0.6230 |
| No change | 0 | 0 | 0 | 0 | 3 | 6.5 | |
| Worsening | | | | | | | |

a: FXFM244 4% vs. FXFM244 1% vs. Placebo;
b: FXFM244 4% vs. Placebo;
c: FXFM244 1% vs. Placebo;
d: FXFM244 4% vs. FXFM244 1%

TABLE 8.5.7
Patient's global improvement assessment after 12 weeks
of treatment by study group. (PP Population)

| Patient global | FXFM244 | | FXFM244 | | | | |
|---|---|---|---|---|---|---|---|
| assessment | 1% | | 4% | | Placebo | | |
| 12 weeks | N = 31 | | N = 36 | | N = 29 | | |
| n | n | % | n | % | n | % | P value |
| Much better | 12 | 38.7 | 20 | 55.5 | 8 | 27.6 | a: 0.2030 |
| Slightly better | 13 | 42.0 | 14 | 38.9 | 14 | 48.3 | b: 0.0460* |
| Same | 4 | 12.9 | 1 | 2.8 | 5 | 17.2 | c: 0.7930 |
| Worse | 1 | 3.2 | 1 | 2.8 | 2 | 6.9 | d: 0.2990 |
| No data | 1 | 3.2 | 0 | 0 | 0 | 0 | |

a: FXFM244 4% vs. FXFM244 1% vs. Placebo;
b: FXFM244 4% vs. Placebo;
c: FXFM244 1% vs. Placebo;
d: FXFM244 4% vs. FXFM244 1%.

The vehicle foam achieved a decrease of about 30% from baseline in the IGA score at about 12 weeks.

After three weeks of treatment no appreciable difference between the three treatments was found.

The above results show that patients who received a vehicle foam showed a percentage of patients with IGA “almost clear” or “clear” of about 20% or more starting from week 6, reaching 33% at week 16, which is better that the results obtained by oral treatments with minocycline (˜17% at week 12), according to the information published on the prescription leaflets of commercially available oral minocycline treatments. Surprisingly, the placebo had a marked positive effect on the percentage of patients with IGA “almost clear” or “clear”.

The percentage of patients receiving vehicle foam with an IGA change of at least 2 units was about 17% or more starting from week 9.

Approximately a third of subjects who received vehicle foam had ‘excellent’ improvement and approximately 60% of subjects had ‘excellent’ or ‘moderate’ improvement as assessed by the physician after twelve weeks of treatment (See Table 8.5.6). Approximately 28% of subjects who received the vehicle foam evaluated their acne as “much better than prior to study”, and over 75% who received the vehicle foam evaluated their acne as “slightly better than prior to study”.

### Efficacy Conclusion

The baseline severity of acne in this study was moderate to severe, as judged by the number of acne lesions and investigator's global severity assessment (IGA). The mean number of inflammatory acne lesions at baseline was 35, and the mean number of non-inflammatory lesions was 45. These mean numbers were much higher than the minimum eligibility criteria of 20 inflammatory and 25 non-inflammatory lesions. The IGA score as assessed by the investigator at baseline was 3.3-3.4, indicating moderate to severe acne at baseline.

Daily application of vehicle foam on facial skin with moderate to severe acne resulted in an improvement of the disease as indicated by the primary and secondary endpoints of the study. There was a reduction in the number of inflammatory, non-inflammatory and sum (total) acne lesions as well as an improvement in the investigator global assessment of acne severity after 12 treatment weeks.

### 8.6. Safety and Tolerability

Generally, the vehicle, the Minocycline Foam 1% and Minocycline Foam 4% were safe and very well tolerated. Very few skin irritation events were recorded, and they were all transient. The severity of these events was primarily mild

### Safety

All adverse events were considered transient and mild in severity. None were considered to have been related to study treatment and none were considered serious adverse events. There were no notable differences between study groups.

There were no notable abnormalities in physical examination of selected body systems (heart, lungs and abdomen) or differences between subjects receiving 1% and 4% minocycline foams, or relative to Placebo (p>0.50); and all were normal by study end. Throughout all visits there were no significant differences observed in nearly all the vital signs (p>0.05), with the exception of heart rate on visits 2 and 6 (p<0.05) between the three study groups receiving 4% or 1% minocycline foam and Placebo in all visits. Post-baseline heart rate values were within normal limits in all subjects in both 1% and 4% groups; however isolated cases of elevated heart rate values were observed in the Placebo group. There were no adverse events associated with vital signs. There were no cumulative changes in vital signs between time points or overall. All vital sign values were normal or within the subject's normal range at the final evaluation. There were no vital sign abnormalities considered clinically significant.

There were 18/139 subjects (12.9%) receiving 19 concomitant medications. Most received only one, 17/139 (12.2%), with one subject receiving two medications. Nearly all concomitant medications were reported at baseline visit 1, 18/19, with only one reported at visit 3. There was no notable trend in the types of medications reported. None of these medications were considered adverse event.

### Tolerability

Tolerability of study treatment was evaluated using the following parameters: erythema, dryness, pigmentation, peeling, and itching using the scale in section 4 above. The score recorded was the most severe irritation score from all 5 parts of the face evaluated.

Overall, no statistically significant differences were demonstrated between treatment groups in any of the selected skin tolerance parameters during the course of the present study (p≧0.1970) (see Table 8.6.1 below).

The vehicle foam was highly tolerated during twelve weeks of treatment. Very few skin irritation events (for example pigmentation, erythema, peeling, itching and dryness) were recorded, and they were all transient. The severity of these events was primarily mild. Specifically, four vehicle patients recorded erythema (mostly mild) during the twelve weeks. One vehicle patient recorded mild pigmentation at visit 3, however the investigator described this case as localized post inflammatory pigmentation, typical to the natural healing process of acne lesions. No cases of itching related to the study was demonstrated through the study. One case of itching linked to herpes simplex was classified as an adverse event and was determined unrelated to the study.

Two patients recorded dryness or peeling after treatment ceased yet did not experience such symptoms during treatment. These post treatment symptoms cannot be directly linked to the placebo but without being bound by any theory may be a result of withdrawal of e.g. oil provided by the formulation.

Thus, it may be concluded that vehicle administered once daily prior to bedtime was safe and well-tolerated. This enhanced safety and tolerability of vehicle is an extremely advantageous feature for medication requiring a prolonged treatment regime. Moreover, the lack of adverse events and systemic side effects avoids the need of constant monitoring of side effects, as required with oral minocycline. It is thus conceivable the vehicle is indeed safer and may be at least if not more effective than oral minocycline or other available topical formulation. The occurrence of skin irritation events is summarized in Table 8.6.1.

| | FXFM244 | FXFM244 | | |
|---|---|---|---|---|
| Tolerability | 1% | 4% | Placebo | ^{a}P value |

Erythema

| | | | | | |
|---|---|---|---|---|---|
| Baseline | n | 46 | 47 | 46 | 0.6220 |
| | No. of none | 44 | 46 | 46 | |
| | No. of mild | 1 | 1 | 0 | |
| | No. of | 1 | 0 | 0 | |
| | moderate | | | | |
| Visit 2 | n | 46 | 47 | 46 | 0.1970 |
| | No. of none | 45 | 43 | 44 | |
| | No. of mild | 0 | 4 | 1 | |
| | No. of | 1 | 0 | 1 | |
| | moderate | | | | |
| Visit 3 | n | 42 | 44 | 41 | 0.5240 |
| | No. of none | 39 | 43 | 39 | |
| | No. of mild | 3 | 1 | 2 | |
| Visit 4 | n | 37 | 41 | 35 | 1.0000 |
| | No. of none | 36 | 40 | 35 | |
| | No. of mild | 1 | 1 | 0 | |
| Visit 5 | n | 36 | 41 | 36 | 1.0000 |
| | No. of none | 35 | 40 | 36 | |
| | No. of mild | 1 | 1 | 0 | |
| Visit 6 | n | 28 | 38 | 31 | 1.0000 |
| | No. of none | 28 | 38 | 31 | |

Dryness

| | | | | | |
|---|---|---|---|---|---|
| Baseline | n | 46 | 47 | 46 | 1.0000 |
| | No. of none | 46 | 47 | 46 | |
| Visit 2 | n | 46 | 47 | 46 | 1.0000 |
| | No. of none | 46 | 47 | 46 | |
| Visit 3 | n | 42 | 44 | 41 | 1.0000 |
| | No. of none | 42 | 44 | 41 | |
| Visit 4 | n | 37 | 41 | 35 | 0.1990 |
| | No. of none | 35 | 41 | 35 | |
| | No. of mild | 2 | 0 | 0 | |
| Visit 5 | n | 36 | 41 | 36 | 1.0000 |
| | No. of none | 36 | 41 | 36 | |
| Visit 6 | n | 28 | 38 | 31 | 1.0000 |
| | No. of none | 28 | 37 | 30 | |
| | No. of mild | 0 | 1 | 1 | |

Pigmentation

| | | | | | |
|---|---|---|---|---|---|
| Baseline | n | 46 | 47 | 46 | 0.6220 |
| | No. of none | 46 | 47 | 45 | |
| | mild | 0 | 0 | 1 | |
| Visit 2 | n | 46 | 47 | 46 | 0.7890 |
| | none | 42 | 45 | 43 | |
| | mild | 3 | 2 | 3 | |
| | severe | 1 | 0 | 0 | |
| Visit 3 | n | 42 | 44 | 41 | 1.0000 |
| | none | 41 | 43 | 40 | |
| | mild | 1 | 1 | 1 | |
| Visit 4 | n | 37 | 41 | 35 | 0.6370 |
| | none | 36 | 41 | 35 | |
| | mild | 1 | 0 | 0 | |
| Visit 5 | n | 36 | 41 | 36 | 1.0000 |
| | none | 36 | 41 | 36 | |
| Visit 6 | n | 28 | 38 | 31 | 1.0000 |
| | none | 28 | 38 | 31 | |

Peeling

| | | | | | |
|---|---|---|---|---|---|
| Baseline | n | 46 | 47 | 46 | 1.0000 |
| | none | 46 | 47 | 46 | |
| Visit 2 | n | 46 | 47 | 46 | 1.0000 |
| | none | 46 | 47 | 46 | |
| Visit 3 | n | 42 | 44 | 41 | 0.6540 |
| | none | 41 | 44 | 41 | |
| | mild | 1 | 0 | 0 | |
| Visit 4 | n | 37 | 41 | 35 | 0.6370 |
| | none | 36 | 41 | 35 | |
| | mild | 1 | 0 | 0 | |
| Visit 5 | n | 36 | 41 | 36 | 1.0000 |
| | none | 36 | 41 | 36 | |
| Visit 6 | n | 28 | 38 | 31 | 0.7760 |
| | none | 28 | 36 | 30 | |
| | mild | 0 | 2 | 1 | |

Itching

| | | | | | |
|---|---|---|---|---|---|
| Baseline | n | 46 | 47 | 46 | 1.0000 |
| | none | 46 | 47 | 46 | |
| Visit 2 | n | 46 | 47 | 46 | 1.0000 |
| | none | 46 | 47 | 46 | |
| Visit 3 | n | 42 | 44 | 41 | 1.0000 |
| | none | 42 | 44 | 41 | |
| Visit 4 | n | 37 | 41 | 35 | 1.0000 |
| | none | 37 | 41 | 35 | |
| Visit 5 | n | 36 | 41 | 36 | 1.0000 |
| | none | 36 | 41 | 36 | |
| Visit 6 | n | 28 | 38 | 31 | 1.0000 |
| | none | 28 | 38 | 31 | |

p > 0.05 NS;
^{a}FXFM244 4% vs. FXFM244 1% vs. Placebo

### 8.7 Patient's Satisfaction

The degree of satisfaction of the patients from the treatment with the investigated products was evaluated based on patient's questionnaires results. The following scale was used to score patient's satisfaction: 0=Equal/indifferent; 1=Moderate; 2=High; 3=Very High; 4=Extremely high.

Scores of patient satisfaction for the three compositions (MCH 1%, MCH 4% and placebo) before and after treatment demonstrating the assessed satisfaction with the three compositions are shown in Table 8.7.1 after twelve weeks. As demonstrated in Table 8.7.1, satisfaction from vehicle was surprisingly comparable to the compositions containing minocycline with regard to effectiveness on blackheads and comparability with former topical drugs.

### 8.7.1_{—} Patient's Satisfaction Questionnaires

Performance

| | Was it | Was it | | Compare with |
|---|---|---|---|---|
| | effective? | effective on | Overall | former topical |
| | Mean values | blackheads? | satisfaction | drugs |
| Placebo | 2.0 | 1.8 | 1.8 | 2.2 |
| MCH 1% | 2.1 | 1.4 | 2.2 | 2.1 |
| MCH 4% | 2.8 | 1.8 | 2.8 | 2.8 |

8.8 Literature Comparison with Current Anti-Acne Drugs

The percentage of the reduction in the number of inflammatory and non-inflammatory lesions after twelve weeks of treatment vehicle of the present invention was compared to the percentage of the reduction in the number of inflammatory and non-inflammatory lesions of using prior art treatments.

Oral minocycline is a well-recognized drug and a standard therapy in the treatment of acne. It was therefore quite surprising that vehicle of the present application which is essentially free of pharmaceutically active agents showed comparable or even better results. Moreover according to the Solodyn™ Prescription Instructions oral minocycline administered for twelve weeks affords about 44% reduction of inflammatory lesions and does not affect non-inflammatory lesions. Whereas according to the present invention the vehicle also has an effect on non-inflammatory lesions. A substantial decrease of 50% inflammatory lesions and 49% non-inflammatory lesions was demonstrated after only six weeks of treatment. A substantial decrease of 57% in non-inflammatory lesions was demonstrated after twelve weeks of treatment. Moreover. Oral Minocycline's major disadvantage is its systemic and significant side effects.

The vehicle foam was well tolerated. The results showed only four patients reported erythema's during the 12 weeks of treatment and these were transient and disappeared. No drug related adverse events were reported. Thus the vehicle is characterized by enhanced safety and tolerability.

The following Table 8.8.1 summarizes the decrease of lesion counts for oral minocycline and 4 additional, recently-approved topical products compared to vehicle after 12 weeks of treatment.

TABLE 8.8.1
Lesion counts for placebo composition compared to prior art treatments after 12 weeks of treatment.

| | | | | | Ziana | |
|---|---|---|---|---|---|---|
| | | | Epiduo | Acanya | (Retinoic | Fabior |
| | | Solodyn (oral | (Adapalene + | (Clindamycin + | acid + | (Tazarotene |
| | Placebo | Minocycline) | BPO) | BPO) | Clindamycin) | Foam) |
| % Change in | −51% | −44% | −47% | −55% | −54% | −57% |
| Inflammatory | | | | | | |
| lesions | | | | | | |
| % Change in | −57% | No effect | −49 (%) | −43% | −43% | −55% |
| Non- | | | | | | |
| inflammatory | | | | | | |
| lesions | | | | | | |
| % Patients | 20% | 17% | 30% | 29% | 31% | 29% |
| with IGA | | | | | | |
| “Clear” or | | | | | | |
| “Almost | | | | | | |
| Clear” | | | | | | |

The following are excerpts from the Prescription Information sheets of Solodyne, Epiduo, Acanya and Ziana.

### Solodyn

TABLE 4
Efficacy Results at Week 12

| | Trial 1 | | Trial 2 | |
|---|---|---|---|---|
| | SOLODYN | | SOLODYN | |
| | (1 mg/kg) | Placebo | (1 mg/kg) | Placebo |
| | N = 300 | N = 151 | N = 315 | N = 158 |
| Mean Percent | 43.1% | 31.7% | 45.8% | 30.8% |
| Improvement in | | | | |
| Inflammatory | | | | |
| Lesions | | | | |
| No. (%) of | 52 (17.3%) | 12 (7.9%) | 50 (15.9%) | 15 (9.5%) |
| Subjects Clear | | | | |
| or Almost Clear | | | | |
| on the EGSA* | | | | |

*Evaluator's Global Severity Assessment
SOLODYN did not demonstrate any effect on non-inflammatory lesions (benefit or worsening).

Epiduo
Study1

| | | Adapalene | Benzoyl | |
|---|---|---|---|---|
| | | 0.1% in | Peroxide | |
| | EPIDUO | Vehicle | 2.5% in | Vehicle |
| | gel | gel | Vehicle gel | gel |
| | (N = 149) | (N = 148) | (N = 149) | (N = 71) |
| IGA: Two Grade | 32 | 18 | 18 | 4 |
| Improvement and | (21.5%) | (12.2%) | (12.1%) | (5.6%) |
| Clear or Almost Clear | | | | |
| Inflammatory Lesions: | 16.0 | 11.4 | 10.5 | 9.5 |
| Mean Absolute | (52.4%) | (39.9%) | (35.8%) | (31.8%) |
| (Percent) Change | | | | |
| Non-inflammatory | 23.4 | 15.2 | 13.7 | 13.2 |
| Lesions: Mean | (45.9%) | (29.6%) | (32.2%) | (27.8%) |
| Absolute (Percent) | | | | |
| Change | | | | |

Study 2

| | | Adapalene | Benzoyl | |
|---|---|---|---|---|
| | | 0.1% in | Peroxide | |
| | EPIDUO | Vehicle | 2.5% in | Vehicle |
| | gel | gel | Vehicle gel | gel |
| | (N = 415) | (N = 420) | (N = 415) | (N = 418) |
| IGA: Two Grade | 125 | 83 | 92 | 47 |
| Improvement and | (30.1%) | (19.8%) | (22.2%) | (11.3%) |
| Clear or Almost Clear | | | | |
| Inflammatory Lesions: | 15.4 | 12.3 | 13.7 | 8.7 |
| Mean Absolute | (53.4%) | (41.7%) | (47.6%) | (30.2%) |
| (Percent) Change | | | | |
| Non-inflammatory | 24.6 | 21.0 | 19.2 | 112 |
| Lesions: Mean | (48.1%) | (40.8%) | (37.2%) | (23.2%) |
| Absolute (Percent) | | | | |
| Change | | | | |

Study 3

| | EPIDUO Gel | Vehide Gel |
|---|---|---|
| | N = 142 | N = 143 |
| IGA: Two Grade | 67 (47.2%) | 22 (15.4%) |
| Improvement and Clear or | | |
| Almost Clear | | |
| Inflammatory Lesions: | 7.4 (36.0%) | 0.7 (−13.2%)* |
| Mean Absolute (Percent) Change | | |
| Non-inflammatory Lesions: | 20.2 (54.7%) | 2.9 (2.3%) |
| Mean Absolute (Percent) Change | | |

In both Studies 1 and 2 the treatment effect was smaller in subjects with a small number of baseline lesions than in subjects with a large number of baseline lesions.
*That is, a mean percent increase of 13.2%

### Acanya Ziana

The results of Study 1 at week 12 are presented in the table below:

| | | | Benzoyl | |
|---|---|---|---|---|
| | ACANYA | Clindamycin | Peroxide | Vehicle |
| | Gel | Gel | Gel | Gel |
| Study 1 | N = 399 | N = 408 | N = 406 | N = 201 |
| EGSS | 115 (29%) | 84 (21%) | 76 (19%) | 29 (14%) |
| Clear or | | | | |
| Almost Clear | | | | |
| 2 grade | 131 (33%) | 100 (25%) | 96 (24%) | 38 (19%) |
| reduction | | | | |
| from baseline | | | | |
| Inflammatory | | | | |
| Lesions: | | | | |
| Mean | 14.8 | 12.2 | 13.0 | 9.0 |
| absolute | | | | |
| change | | | | |
| Mean | 55.0% | 47.1% | 49.3% | 34.5% |
| percent (%) | | | | |
| reduction | | | | |
| Non- | | | | |
| Inflammatory | | | | |
| Lesions: | | | | |
| Mean | 22.1 | 17.9 | 20.6 | 13.2 |
| absolute | | | | |
| change | | | | |
| Mean | 45.3% | 38.0% | 40.2% | 28.6% |
| percent (%) | | | | |
| reduction | | | | |

The results of Study 2 at week 12 are presented in the table below:

| | | | Benzoyl | |
|---|---|---|---|---|
| | ACANYA | Clindamycin | Peroxide | Vehicle |
| | Gel | Gel | Gel | Gel |
| Study 2 | N = 398 | N = 404 | N = 403 | N = 194 |
| EGSS | 113 (28%) | 94 (23%) | 94 (23%) | 21 (11%) |
| Clear or | | | | |
| Almost Clear | | | | |
| 2 grade | 147 (37%) | 114 (28%) | 114 (28%) | 27 (14%) |
| reduction | | | | |
| from baseline | | | | |
| Inflammatory | | | | |
| Lesions: | | | | |
| Mean | 13.7 | 11.3 | 11.2 | 5.7 |
| absolute | | | | |
| change | | | | |
| Mean | 54.2% | 45.3% | 453% | 23.3% |
| percent (%) | | | | |
| reduction | | | | |
| Non- | | | | |
| Inflammatory | | | | |
| Lesions: | | | | |
| Mean | 19.0 | 14.9 | 15.2 | 8.3 |
| absolute | | | | |
| change | | | | |
| Mean | 41.2% | 34.3% | 34.5% | 19.2% |
| percent (%) | | | | |
| reduction | | | | |

TABLE 3
Efficacy Results at Week 12 in Studies 1 and 2.

| | | | |
|---|---|---|---|
| ZIANA Gel | Clindamycin | Tretinoin | Vehicle |
| N = 845 | N = 426 | N = 846 | N = 423 |

Evaluator's Global Severity: N (%)

| | | | | |
|---|---|---|---|---|
| Patients | 180 | 70 | 122 | 34 |
| achieving | (21%) | (16%) | (14%) | (8%) |
| success* | | | | |

Inflammatory Lesion Count (% reduction from baseline)

| | | | | |
|---|---|---|---|---|
| Mean | 48% | 42% | 39% | 26% |

Non-inflammatory Lesion Count (% reduction from baseline)

| | | | | |
|---|---|---|---|---|
| Mean | 36% | 27% | 31% | 16% |

Total Lesion Count (% reduction from baseline)

| | | | | |
|---|---|---|---|---|
| Mean | 41% | 34% | 34% | 20% |

*Success was defined as cleared or almost cleared at Week 12.

TABLE 4
Efficacy Results at Week 12 in Study 3.

| | |
|---|---|
| ZIANA Gel | Clindamycin |
| N = 1008 | N = 1002 |

Evaluator's Global Severity: N (%)

| | | |
|---|---|---|
| Patients | 415 (41%) | 345 (34%) |
| achieving | | |
| success* | | |

Inflammatory Lesion Count (% reduction from baseline)

| | | |
|---|---|---|
| Mean | 61% | 55% |

Non-inflammatory Lesion Count (% reduction from baseline)

| | | |
|---|---|---|
| Mean | 50% | 41% |

Total Lesion Count (% reduction from baseline)

| | | |
|---|---|---|
| Mean | 54% | 47% |

*Success was defined as at least a 2-grade improvement at Week 12 from baseline.

Vehicle foam gave better results than Epiduo™, Acanya™, Ziana™ and Fabior™ in the percentage of change with respect of non-inflammatory lesions.

### DISCUSSION

Oral minocycline is known to be effective in acne treatment. It is e.g. part of an oral tablet sold under the trademark Solodyn™ and approved by various regulatory authorities including the FDA. Minocycline tablets such as Solodyn™ are therefore considered as a standard therapy in the treatment of acne. Oral minocycline, however, is associated with multiple unwanted side effects. There was until now, no vehicle which is essentially free of pharmaceutically active agents and specifically free of minocycline that is as effective as oral minocycline in the treatment of acne.

Applicants have carried out clinical investigations in a Phase II, placebo controlled and double blinded clinical study in acne patients comparing the vehicle as described herein with compositions which further contain 1% Minocycline or 4% Minocycline and described in Example 2b of the present application.

The results surprisingly demonstrated that the vehicle, which is essentially free of pharmaceutically active agents, administered topically once daily had an unexpected effect in reducing the number of inflammatory and non-inflammatory lesions in patients and was safe and well-tolerated.

A reduction in lesions was observed from about 3 weeks of treatment with the vehicle with improvement continuing to 12 weeks. Non-inflammatory lesions responded, in general, slightly better that than inflammatory lesions. The effect of treatment on reducing the number of both inflammatory and non-inflammatory lesions and improving the patient's skin appeared to approach a steady state between 6-12 weeks. Treatment was stopped at twelve weeks but the patients were seen again 4 weeks after cessation of treatment at week 16. Surprisingly, the effect of the previous 12 weeks of treatment on reducing the number of lesions and improving the patient's skin was observed to continue in the absence of treatment with minor decrease in the mean number of lesions. In other words, four weeks after cessation the patient's skin did not appear to show signs of relapse.

Surprisingly, topical administration of vehicle foam, once daily provided effective treatment to an infected lesion site, leading to rapid reduction of about 50% in the number of non-inflammatory and inflammatory acne lesions within only six weeks of treatment. A further reduction of about 60% in the number of non-inflammatory lesions was observed after twelve weeks of treatment. The improvement indicated in the Investigators Global Improvement Assessment of the vehicle was also very encouraging and even four weeks after treatment ceased patients IGA score continued to improve.

The percentage of patients, for example, with an IGA at 12 weeks of “almost clear” or “clear” was 20% for the placebo. Even after treatment ceased patients IGA score continued to improve to 33%. Patient feedback and overall patient satisfaction was likewise positive.

Even more surprisingly, the improvement in inflammatory lesions observed with the vehicle was not merely favorably comparable to the efficacy results for oral minocycline as presented in the Solydyn™ Patient Product leaflet at 12 weeks but had even better efficacy as early as 6 weeks.

The reduction in non-inflammatory lesions demonstrated with the vehicle is greater than for four recently approved topical products which use active ingredients other than tetracycline antibiotics, namely Epiduo™, Acanya™ Fabior™ and Ziana™. So apart from the avoidance of unwanted systemic effects, topical vehicle treatment appears to have substantial advantages over oral minocycline treatment of acne and other available topical treatments. After only six weeks of treatment with the vehicle patients showed significantly less symptoms of acne. The symptoms further decreased over time upon continuation of the application as described above. Patients with both inflammatory and non-inflammatory forms of acne benefited from the application of the vehicle, however non-inflammatory lesion seemed to benefit even more.

Remarkably, only few skin irritation events were observed in patients treated with the vehicle during 12 weeks of treatment. All these skin irritation events were transient and did not require discontinuance treatment. There was no difference in their occurrence among the study groups. No itching or pigmentation related to the drug was observed throughout the study.

It has surprisingly been noted that a vehicle directed to the treatment of acne vulgaris having a waterless oily carrier, comprising foam adjuvants has a skin conditioning effect as well as a therapeutic effect which results in restoration of skin integrity, freshness and supple appearance. The fact that the compositions are also free of surfactants and alcohols which are known skin irritants make these formulations suitable for sensitive and damaged skin. Moreover, possible anti-inflammatory attributes of the vehicle seem to be indicated by the lack of pigmentation and itching.

The claimed vehicle penetrates the skin. Without being bound by any theory the following observations may be noted. The reduction in inflammatory lesions may be a result of the vehicle killing bacteria and or preventing growth of new bacteria. The vehicle was shown to heal blackheads and blemishes and keep skin healthy, thereby perhaps preventing future acne, and appears to have skin healing properties. The vehicle may modulate the inflammatory response to reduce redness, swelling and scarring. The vehicle offers significant advantages over available topical compositions as discussed in detail before.

Thus, the topical vehicle offers an easy, safe and an effective alternative for the oral and/or topical treatment of acne vulgaris. The ease of use, with once daily dosing, as well as its broad spectrum of activity treating both inflammatory and non-inflammatory lesions, quick onset of clinical effect, high tolerability and the lack of serious adverse events, or drug related adverse events make it an attractive choice. Enhanced efficacy and safety of the vehicle of a single dose each day for only six weeks indicate that a short single daily regime was also effective improving patient compliance. In addition to application as a foam the results can be extrapolated for use with gel and liquid gel delivery formats.

1. (canceled)

2. A method for the treatment, alleviation, or prophylaxis of a skin or mucosa disorder comprising topically administering on at least alternate days or at least once daily to a target area on a human having the disorder, a waterless hydrophobic gel or foam composition comprising:
a) about 60% to about 99% by weight of the composition of a minocycline compatible hydrophobic solvent;
b) about 0.1% to about 20% by weight of the composition of a minocycline compatible excipient selected from the group consisting of a wax, a fatty alcohol or a fatty acid, and mixtures of any two or more thereof;
wherein the wax, if present, is selected from a group consisting of a hydrogenated castor oil, a beeswax, a paraffin wax, a wax that is solid at room temperature, and mixtures of any two or more thereof;
wherein the fatty alcohol, if present, has a carbon chain length of at least 12 to 22 carbons;
wherein the fatty acid, if present, has a carbon chain length of at least 12 carbons;
wherein the composition is essentially free of pharmaceutically active agents;
wherein the minocycline compatible hydrophobic solvent and/or excipient has therapeutic effect topically against the disorder;
wherein if the waterless hydrophobic gel composition is packaged in a canister with an outlet valve to which is added a liquefied or compressed gas propellant the composition affords upon release from the canister a breakable hydrophobic foam.

3. The method of claim 2, wherein the ratio of gel composition to propellant is from about 100:3 to 100:30, and wherein the breakable foam has a collapse time of at least 180 seconds at 36° C.

4. The method of claim 2, wherein the disorder is selected from the group consisting of acne, acne conglobate, acne fulminans, acne vulgaris, nodular papulopustar acne, acne related symptoms, a tetracycline antibiotic responsive acne related disorder, a skin disorder caused by a bacteria, a tetracycline antibiotic responsive sebaceous gland disease, P. acne bacteria associated disorders, superficial infections, skin rosacea, atopic dermatitis, contact dermatitis, perioral dermatitis, psoriasis, neurodermitis, and any two or more thereof.

5. The method of claim 2, wherein the fatty alcohol, if present, comprises one or more of myristyl alcohol, cetyl alcohol, stearyl alcohol, cetostearyl alcohol, and behenyl alcohol; and wherein the fatty acid, if present, comprises stearic acid.

6. The method of claim 5, wherein the hydrophobic solvent is selected from a group consisting of an essential oil, a therapeutic oil, an emollient, a silicone oil, an oil of plant origin, a hydrocarbon oil, an oil from animal origin, an unsaturated or polyunsaturated oil, a mineral oil, an ester oil, an ester of a dicarboxylic acid, a triglyceride oil, an oil from animal origin, an unsaturated or polyunsaturated oil, a diglyceride, a PPG alkyl ether, liquid paraffin, an isoparaffin, a polyalphaolefin, a polyolefin, polyisobutylene, a synthetic isoalkane, isohexadecane, isododecane, an alkyl benzoate, an alkyl octanoate, a C12-C15 alkyl benzoate, a C12-C15 alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, an alexandria laurel tree oil, an avocado oil, an apricot stone oil, a barley oil, a borage seed oil, a calendula oil, a canelle nut tree oil, a canola oil, caprylic/capric triglycerides, a castor oil, a coconut oil, a corn oil, a cotton oil, a cottonseed oil, an evening primrose oil, a flaxseed oil, a groundnut oil, a hazelnut oil, glycereth triacetate, glycerol triheptanoate, glyceryl trioctanoate, glyceryl triundecanoate, a hempseed oil, a jojoba oil, a lucerne oil, a maize germ oil, a MCT oil, a marrow oil, a millet oil, neopentylglycol dicaprylate/dicaprate, an olive oil, a palm oil, a passionflower oil, pentaerythrityl tetrastearate, a poppy oil, propylene glycol ricinoleate, a rapeseed oil, a rye oil, a safflower oil, a sesame oil, a shea butter, a soya oil, a sweet almond oil, a sunflower oil, a sysymbrium oil, a syzigium aromaticum oil, a tea tree oil, a walnut oil, wheat germ glycerides, a wheat germ oil, a PPG-2 butyl ether, a PPG-4 butyl ether, a PPG-5 butyl ether, a PPG-9 butyl ether, a PPG-12 butyl ether, a PPG-14 butyl ether, a PPG-15 butyl ether, a PPG-15 stearyl ether, a PPG-16 butyl ether, a PPG-17 butyl ether, a PPG-18 butyl ether, a PPG-20 butyl ether, a PPG-22 butyl ether, a PPG-24 butyl ether, a PPG-26 butyl ether, a PPG-30 butyl ether, a PPG-33 butyl ether, a PPG-40 butyl ether, a PPG-52 butyl ether, a PPG-53 butyl ether, a PPG-10 cetyl ether, a PPG-28 cetyl ether, a PPG-30 cetyl ether, a PPG-50 cetyl ether, a PPG-30 isocetyl ether, a PPG-4 lauryl ether, a PPG-7 lauryl ether, a PPG-2 methyl ether, a PPG-3 methyl ether, a PPG-3 myristyl ether, a PPG-4 myristyl ether, a PPG-10 oleyl ether, a PPG-20 oleyl ether, a PPG-23 oleyl ether, a PPG-30 oleyl ether, a PPG-37 oleyl ether, a PPG-40 butyl ether, a PPG-50 oleyl ether, a PPG-11 stearyl ether, a herring oil, a cod-liver oil, a salmon oil, a cyclomethicone, a dimethyl polysiloxane, a dimethicone, an epoxy-modified silicone oil, a fatty acid-modified silicone oil, a fluoro group-modified silicone oil, a methylphenylpolysiloxane, a phenyl trimethicone, a polyether group-modified silicone oil.

7. The method of claim 2, wherein the hydrophobic solvent is selected from the group consisting of an essential oil, a therapeutic oil, an emollient, a silicone oil, an oil of plant origin, or hydrocarbon oil or mixtures of any two or more thereof.

8. The method of claim 2, wherein the hydrophobic solvent is selected from the group consisting of a mineral oil, a soybean oil, a coconut oil, a silicone oil, or mixtures of any two or more thereof.

9. The method of claim 5, wherein the wax and/or fatty alcohol and/or fatty acid are about 0.4% to about 18% by weight of the composition.

10. The method of claim 5, wherein the composition further comprises a fumed silica.

11. The method of claim 5, wherein each excipient or hydrophobic solvent used in the gel or foam composition is selected on the basis that when an excipient or hydrophobic solvent is mixed with minocycline, the minocycline does not break down more than 10%.

12. The method of claim 5, wherein the composition is non-irritating and suitable for ophthalmic use or for use on other sensitive targets.

13. The method of claim 4, wherein the disorder is a skin inflammation or acne or damage to skin induced by a cause selected from the group consisting of radiation, cold, burns, chemical burns, and any two or more thereof.

14. The method of claim 2, wherein the composition is free of a substance selected from the group consisting of a surfactant, a polymeric gelling agent, a polyol, a petrolatum, protic solvents, polar aprotic solvents, isopropyl myristate, polyethylene gelling agents, polyethylene homopolymers, polyethylene copolymers, selenium derivatives, silicone thickening agents, elastomers, a hydrophilic agent, a short chain alcohol, ethanol, propanol, butanol, pentanol, pomegranate seed oil, an ethoxylated lanolin oil derivative, a lanolin oil and any two or more thereof.

15. The method according to claim 2, wherein said composition consists of:
a) about 50% by weight of a soybean oil;
b) about 23% by weight of a coconut oil;
c) about 5% by weight of a cyclomethicone;
d) about 6% by weight of a light mineral oil;
e) about 3.5% by weight of cetostearyl alcohol;
f) about 3% by weight of stearic acid;
g) about 2.5% of myristyl alcohol;
h) about 2% by weight of a hydrogenated castor oil;
i) about 2% by weight of a beeswax;
j) about 1.5% by weight of stearyl alcohol; and
k) about 1.1% by weight of behenyl alcohol;

16. The method of claim 2, wherein the composition further comprises a color agent, a cosmetic agent, or a mixture thereof; wherein the color agent or cosmetic agent is suspended in the composition; wherein the color agent is selected from the group consisting of D&C No. 10, D&C No. 11, and a mixture thereof, and
wherein the color agent is present at about 0.0001% to about 0.1% by weight of the composition.

17. The method of claim 3, wherein the propellant is selected from the group consisting of butane, propane, isobutene, a dimethylether, a fluorocarbon, or mixtures of two or more thereof.

18. The method of claim 15, wherein the disorder is acne and wherein the reduction in lesion count is at least about 45% or more than 45% compared to the number of lesions immediately prior to treatment and wherein the hydrophobic gel or foam is safe and tolerated when the hydrophobic gel or foam composition is administered at least once daily for a period of at least six weeks.

19. The method of claim 15, wherein the hydrophobic gel or foam composition is applied at a frequency selected from the group consisting of three times daily, twice daily, and once daily, and wherein the hydrophobic gel or foam composition is administered for a period selected from the group consisting of two weeks, three weeks, four weeks, five weeks, six weeks, seven weeks, eight weeks, nine weeks, ten weeks, eleven weeks, twelve weeks, thirteen weeks, fourteen weeks, fifteen weeks and sixteen weeks.

20. The method of claim 15, wherein the disorder is acne and wherein a decrease in the number of inflammatory acne lesions after 6 weeks of treatment compared to the number of lesions immediately prior to treatment is at least about 45%, wherein the hydrophobic foam composition or gel is administered once daily.

21. The method of claim 15, wherein the disorder is acne and wherein decrease in non-inflammatory acne lesions after 6 weeks of treatment compared to the number of lesions immediately prior to treatment is at least about 45%, wherein the hydrophobic foam composition or gel is administered once daily.

22. The method of claim 15, wherein the disorder is acne and wherein decrease in non-inflammatory or inflammatory acne lesions or total lesions after 12 weeks of treatment compared to the number of lesions immediately prior to treatment is at least about 50%, wherein the hydrophobic foam composition or gel is administered once daily.

23. The method of claim 15, wherein the disorder is acne and wherein the number of non-inflammatory acne lesions or inflammatory acne lesions or total acne lesions is reduced by at least 30% or more than 30% after 3 weeks of treatment compared to the number of lesions immediately prior to treatment.

24. The method of claim 15, wherein the disorder is acne and wherein said method is essentially free of skin irritation and adverse events or is free of serious adverse events.

25. The method of claim 15, wherein the disorder is acne and wherein the percentage of patients with an IGA of “Clear” or “Almost Clear” is higher for patients treated with the hydrophobic foam composition or gel in comparison with oral minocycline after 12 weeks of treatment compared to the number of lesions immediately prior to treatment.

26. A method for the treatment, alleviation, or prophylaxis of a skin or mucosa disorder comprising topically administering on at least alternate days or at least once daily to a target area of a subject having the disorder, a waterless hydrophobic vehicle in the form of a gel or foam composition comprising:
a) about 72% to about 88% by weight of at least one a minocycline compatible hydrophobic solvent comprising;
b) about 10% to about 22% by weight of the of a minocycline compatible excipient comprising a wax and/or a fatty alcohol, and/or a fatty acid;
c) about 2% to about 6% of a color agent and/or a cosmetic agent;
wherein the minocycline compatible hydrophobic solvent and/or excipient has therapeutic effect topically against the disorder;
wherein the wax, if present, comprises a hydrogenated castor oil, a beeswax, or both;
wherein the fatty alcohol, if present, comprises stearyl alcohol;
wherein the fatty acid, if present, comprises stearic acid;
wherein the disorder is selected from the group consisting of acne, acne related symptoms, a tetracycline antibiotic responsive acne related disorder, a skin disorder caused by a bacteria, a tetracycline antibiotic responsive sebaceous gland disease, P. acne bacteria associated disorders, superficial infections, skin rosacea, atopic dermatitis, contact dermatitis, perioral dermatitis, psoriasis, neurodermitis, and any two or more thereof;
wherein if the gel is packaged in a canister with an outlet valve to which is added a liquefied or compressed gas propellant the composition affords upon release from the canister a breakable hydrophobic foam.
